(19) United States
(12) Patent Application Publication
(10) Pub. No.: US 2008/0248565 A1
(43) Pub. Date: Oct. 9, 2008

(54) ISOLATED PHOSPHOLIPID-PROTEIN PARTICLES

(75) Inventors: Federico Katzen, Carlsbad, CA (US); Julia Fletcher, Carlsbad, CA (US); Wieslaw Kudlicki, Carlsbad, CA (US); Joseph Beechem, Eugene, OR (US); Lilin Wang, San Diego, CA (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 12/040,798

(22) Filed: Feb. 29, 2008

Related U.S. Application Data
(60) Provisional application No. 60/892,525, filed on Mar. 1, 2007.
(60) Provisional application No. 60/908,678, filed on Mar. 28, 2007.
(60) Provisional application No. 60/910,209, filed on Apr. 4, 2007.
(60) Provisional application No. 60/910,211, filed on Apr. 5, 2007.

(51) Int. Cl. C12N 5/06; C07K 14/00

(52) U.S. Cl. 435/325; 530/359

(57) ABSTRACT

Systems and methods are provided for producing a protein of interest that is typically not amenable to expression in soluble form in in vitro expression systems. In some aspects, the invention provides methods of synthesizing proteins using in vitro protein synthesis systems that include a scaffold protein such as apolipoprotein or an amphipathic alpha helix containing (“AAHC”) protein, in which higher yields of soluble protein are produced than in the absence of the scaffold protein. The scaffold proteins may be provided in an in vitro protein synthesis system associated with lipid or not associated with lipid. The scaffold protein may be provided as a protein per se or may be encoded by a nucleic acid template and co-expressed with the protein of interest. The invention also provides compositions and kits for synthesis of proteins in soluble form, in which the compositions and kits include cell extracts for protein expression and isolation.

### PRIORITY

This application claims priority to U.S. Provisional Application Ser. Nos. 60/892,525 filed Mar. 1, 2007; 60/908,678 filed Mar. 28, 2007; 60/910,209 filed Apr. 4, 2007; and, 60/910,211 filed Apr. 5, 2007.

### SEQUENCE LISTING

The instant application contains a Sequence Listing which is hereby incorporated by reference in its entirety.

### FIELD OF THE INVENTION

The invention relates generally to in vitro protein synthesis systems and more specifically to in vitro translation of membrane proteins and hydrophobic proteins.

### BACKGROUND INFORMATION

Strategies for treating medical conditions such as aging-related disorders, autoimmune diseases, and cancer rely heavily on understanding protein function. The majority of drug targets are proteins, and it is thought that at least half of protein drug targets are membrane proteins. The ability to efficiently synthesize proteins, and particularly membrane proteins, in amounts that can be used for studies of structure and function is critical to the discovery of new drugs that can combat disease.

In vitro protein synthesis systems, in which proteins can be made from a nucleic acid template in a cell free extract, allowing for efficient synthesis and subsequent isolation of proteins, can allow for high throughput structural and functional analysis of proteins that can accelerate research and drug discovery efforts in particular.

Unfortunately, not all proteins are synthesized in soluble form in in vitro synthesis systems. Membrane proteins in particular are often insoluble when produced in cell-free translation system, making it necessary to solubilize the proteins, often in denaturing detergents and then attempt to renature the proteins to investigate their native structure and activity. These endeavors are laborious and often unsuccessful.

Bayburt et al. have described the spontaneous formation of nanoscale lipid-protein particles when detergent solubilized apolipoprotein A1 (“Apo A1”) and phospholipids are mixed (Bayburt, T. H., Carlson, J. W., and Sligar, S. G. (1998) “Reconstitution and Imaging of a Membrane Protein in a Nanometer-Sized Phospholipid Bilayer.” Journal of Structural Biology, 123, 37-44.) Dialyzing away the detergent leaves nanoscale lipid-protein particles that, by structural analysis have been determined to be composed of a lipid bilayer encircled by the Apo A1 protein. Bayburt and Sligar have described synthetic variants of Apo A1 (“scaffold proteins”) that behave like Apo A1 in forming lipid-protein particles in the presence of detergent. (Civjan, N. R., Bayburt, T. H., Schuler, M. A., and Sligar, S. G. (2003) “Direct Solubilization of Heterologously Expressed Membrane Proteins by Incorporation into Nanoscale Lipid Bilayers.” BioTechniques, 35, 556-563; U.S. Pat. No. 7,048,949; U.S. Pat. No. 7,083,958; and U.S. Patent Application Publication No. 2005/0182243, all of which are herein incorporated by reference in their entireties. These researchers have found that other membrane proteins, when solubilized with detergent, will incorporate into the lipid bilayer of the nanodiscs if provided in the same self-assembly detergent mix and then subjected to dialysis.

This technology for providing a membrane protein in soluble form however still requires a large effort in purifying and solubilizing the membrane protein before it is combined with the nanodisc components in the self-assembly detergent mix. These processes must be individualized for particular proteins, are time-consuming and labor-intensive, and often require the use of harsh denaturing reagents that can affect protein function. Thus, a need exists for a convenient method of expressing membrane proteins in in vitro systems that provide the protein in a soluble, native, and substantially purified or readily purifiable form using faster procedures.

### SUMMARY OF THE INVENTION

Described herein are compositions and methods for the in vitro synthesis of one or more proteins of interest (POI) in the presence of one or more “scaffold proteins” having one or more amphipathic alpha helices such that the POI and the scaffold protein form a complex that improves the solubility of the POI. In certain embodiments, a phospholipid is also included such that the POI, scaffold protein, and phospholipid form phospholipid protein particles (PPPs). In certain embodiments, the POI is encoded by a nucleic acid. It may be desired to complex the phospholipid and scaffold protein prior to expression of the POI such that it is expressed in the presence of the phospholipid-scaffold protein complex. The POI and scaffold protein may also be encoded on the same or separate nucleic acids and co-expressed in the in vitro synthesis system, either in the presence or absence of phospholipids. The phospholipid-scaffold protein complex may also be referred to as a PPP; thus, a PPP requires at a minimum a phospholipid and a scaffold protein.

In certain embodiments, a phospholipid is utilized. Suitable phospholipids are any capable of forming a phospholipid bilayer into which a scaffold protein and/or POI may be incorporated. Many suitable phospholipids are known in the art. Exemplary phospholipids include but are not limited to phosphatidyl choline, phosphatidyl ethanolamine, phosphatidyl inositol, dipalmitoyl-phosphatidylcholine, dimyristoyl phosphatidyl choline, 1-palmitoyl-2-oleoyl-phosphatidyl choline, dihexanoyl phosphatidyl choline, dipalmitoyl phosphatidyl ethanolamine, dipalmitoyl phosphatidyl inositol, dimyristoyl phosphatidyl ethanolamine, dimyristoyl phosphatidyl inositol, dihexanoyl phosphatidyl ethanolamine, dihexanoyl phosphatidyl inositol, 1-palmitoyl-2-oleoyl-phosphatidyl ethanolamine, and 1-palmitoyl-2-oleoyl-phosphatidyl inositol.

A scaffold protein is typically utilized, with or without one or more phospholipids. A suitable scaffold protein is one that is capable of associating with a POI to improve its solubility, and in certain embodiments is also capable of associating with a phospholipid bilayer. It is preferred that association of a scaffold protein with a POI, with or without phospholipids, increase the solubility of the POI translated in the IVPS system by at least 10%, 15%, 20%, or 25% over the solubility of the POI produced in the IVPS system in the absence of the scaffold protein. Solubility may be measured by any known technique including, as shown herein, gel electrophoresis. Preferred scaffold proteins are proteins that associate with lipids, preferably phospholipids, and include at least one amphipathic alpha helix (“amphipathic alpha helix containing protein” or “AAHC”). As described herein, in certain embodiments, the scaffold protein is an apolipoprotein. Exemplary scaffold proteins include, for example, apolipoproteins such as Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, Lipoprotein (a), Apoliphorin I, Apoliphorin II, Apoliphorin III; MSP1; synucleins (e.g., synuclein alpha (e.g., NM007308 (SEQ ID NO:84) or NM000345 (SEQ ID NO:85), synuclein beta (NM001001502 (SEQ ID NO:86) or NM003085 (SEQ ID NO:87), or gamma (NM003087; SEQ ID NO:88), apomyoglobin; or, peptabiols such as, for example, melitin, almethicin, or a gramicidin; or any variants thereof. Variants of naturally-occurring scaffold proteins may be utilized. For instance, in certain embodiments, a scaffold protein may include an amphipathic alpha helix that is approximately 70, 80, 90, 95 or 99% identical to at least, for example, approximately 10 or 15 amino acids of any of the exemplary scaffold proteins described herein. The scaffold protein may have an amino acid sequence that is modified with respect to the amino acid sequence of a wild-type protein by having one or more amino acid deletions, insertions, or substitutions. The scaffold protein may include one or more chemical or enzymatic modifications, and/or a label or tag, such as a peptide tag. In certain embodiments, such labels or tags are detectable and/or useful for isolating the POI associated with the scaffold protein (e.g., the POI and scaffold proteins co-associate). The terms scaffold protein, “protein that comprises one or more amphipathic alpha helices”, “amphipathic alpha helix containing protein” (“AAHC”) protein” are interchangeable within this disclosure.

A suitable POI is a hydrophobic protein that is not typically expressible at high levels in a soluble form. For example, membrane proteins are often difficult to isolate using bacterial (e.g., E. coli) expression systems. Many such proteins are known in the art. In certain embodiments, such proteins include but are not limited to enzymes, structural proteins, carrier proteins, transporters, receptors (e.g., a G protein-coupled receptor, a tyrosine kinase receptor, a cytokine receptor, etc.), ion channel proteins, G proteins, pore-forming proteins, adhesion proteins (e.g., a cell adhesion molecule (CAM) or substrate adhesion molecule (SAM)), hormones, growth factors, inhibitors, or activators. Additional non-limiting examples include bacterial membrane protein, EmrE (SEQ ID NO: 43), bacteriorhodopsin (SEQ ID NO: 44), a polypeptide expressible from the Invitrogen Ultimate™ ORF clone collection, a G protein-coupled receptor (GPCR), G protein-coupled receptor family C group 5 member C (NM_{—}022036; SEQ ID NO: 45), G protein-coupled receptor 157 (BC018691.1; SEQ ID NO: 46), serotonin receptor HTR1 (IOH46452; SEQ ID NO: 47), endothelin receptor type B (NM_{—}000115.1; SEQ ID NO: 48), opiate receptor-like 1 (NM_{—}000913.3; SEQ ID NO: 50), cholinergic receptor muscarinic 2 (NM_{—}000739.2; SEQ ID NO: 50), histamine receptor H2 (BC054510.2; SEQ ID NO: 51), dopamine receptor D1 (NM_{—}000794.3; SEQ ID NO: 52), melanocortin 5 receptor (NM_{—}005913.1; SEQ ID NO: 53), corticotropin releasing hormone receptor 1 (NM_{—}004382.2; SEQ ID NO: 54), 5-hydroxytryptamine (serotonin) receptor 1A (NM_{—}000524.2; SEQ ID NO: 55), cholinergic receptor muscarinic 1 (NM_{—}000738.2; SEQ ID NO: 56), CD24 (NM_{—}013230.2; SEQ ID NO: 57), glycophorin E (BC017864.1; SEQ ID NO: 58), glycophorin B (NM_{—}002100.3; SEQ ID NO: 59), chemokine-like factor (NM_{—}181640.1; SEQ ID NO: 60), glycophorin A (BC005319.1; SEQ ID NO: 61), murine microsomal glutathione S-transferase 1 (BC009155.1; SEQ ID NO: 62), phosphatidylinositol glycan anchor biosynthesis class P (NM_{—}153681.2; SEQ ID NO: 63), epiregulin (NM_{—}007950.1; SEQ ID NO: 64), epiregulin (NM_{—}001432.2; SEQ ID NO: 65), CD99 (NM_{—}002414.3; SEQ ID NO: 66), murine Mpv17 transgene (NM_{—}008622.2; SEQ ID NO: 67), MpV17 mitochondrial inner membrane protein (NM_{—}002437.4; SEQ ID NO: 68), translocase of inner mitochondrial membrane 22 homolog (NM_{—}013337.2; SEQ ID NO: 69), ninjurin 2 (NM_{—}016533.4; SEQ ID NO: 70), signal peptide peptidase-like 2B (BC001788.1; SEQ ID NO: 71), CKLF-like MARVEL transmembrane domain containing 1 (NM_{—}181268.2; SEQ ID NO: 72), golgi transport 1 homolog B (NM_{—}016072.3; SEQ ID NO: 73), leukotriene C4 synthase (NM_{—}145867.1; SEQ ID NO: 74), angiotensin II receptor-associated protein (NM_{—}001040194.1; SEQ ID NO: 75), arachidonate 5-lipoxygenase-activating protein (NM_{—}001629.2; SEQ ID NO: 76), signal peptide peptidase 3 (NM_{—}025781.1; SEQ ID NO: 77), leptin receptor (NM_{—}017526.2; SEQ ID NO: 78), microsomal glutathione S-transferase 3 (NM_{—}004528.2; SEQ ID NO: 79), dystrobrevin binding protein 1 (NM_{—}033542.2; SEQ ID NO: 80), PRA1 domain family member 2 (NM_{—}007213.1; SEQ ID NO: 81), phosphatidic acid phosphatase type 2 domain containing 1B (NM_{—}032483.3; SEQ ID NO: 82), and human adrenomedullin receptor protein (SEQ ID NO: 83). Fragments or variants of POIs may also be used. As described herein, POIs may also be co-expressed or complexed with other proteins such as chaperonins or subunits normally expressed with the POI in a cell. Functional domains of POIs may also be utilized, either alone or as fusion proteins with other proteins that may serve to anchor the domain within the PPP. POIs may also be used in conjunction with or expressed as fusion proteins with other proteins such as those tagged with, for example, a fluorescent tag (e.g., green fluorescent protein (GFP, EGFP), blue fluorescent protein (BFP, EBFP, EBFP2, Azurite, mKalama1), cyan fluorescent protein (CFP, ECFP, Cerulean, CyPet), red fluorescent protein (RFP), or yellow fluorescent protein (YFP, YFP, Citrine, Venus, YPet) or fluorescent variants thereof with at least 80% sequence identity to a native GFP, EGFP, BFP, CFP, RFP, or YFP) for utilization in detection assays (e.g., FRET assays).

Also provided are methods for producing a POI in soluble form using in vitro expression systems. The method includes adding a nucleic acid template that encodes a POI to an in vitro protein synthesis system in the presence of a scaffold protein, and optionally one or more phospholipids, and incubating the in vitro protein synthesis system under conditions amendable to production of a soluble POI. In certain embodiments, such conditions include but are not limited to the inclusion of a scaffold protein, either as a co-translated expression product of a nucleic acid, or as the protein per se, and optionally the inclusion of one or more phospholipids. The POI and scaffold proteins may be encoded by one or more nucleic acid templates. The nucleic acid templates encoding the POI and scaffold protein may be the same or different. A single nucleic acid template encoding both the POI and the scaffold protein may include separate promoters controlling expression of the POI and the scaffold protein, and/or may include a common promoter along with another element, such as an IRES sequence inserted between the two gene sequences, allowing for expression of both proteins from the same promoter. The nucleic acid template or templates may consist of any type of nucleic acid, such as DNA or RNA. Where multiple templates are utilized, the templates may be different types of nucleic acids. For example, where two templates are utilized, one may be DNA and one may be RNA, or both may be either DNA or RNA. The POI is preferably synthesized in soluble form through its association with the scaffold protein and, in certain embodiments, one or more phospholipids.

In another aspect, the invention provides an in vitro protein synthesis system (“IVPS”) that includes a cell extract, a scaffold protein, and optionally one or more phospholipids. Cell extracts that include components of the protein synthesis machinery are well-known in the art, and can be from prokaryotic or eukaryotic cells. The in vitro protein synthesis system can further include one or more nucleic acid templates. In one embodiment, an in vitro protein synthesis system including a cell extract, a nucleic acid template encoding a scaffold protein, a nucleic acid template encoding a POI, and optionally one or more phospholipids is provided. In other embodiments, an in vitro protein synthesis system including a cell extract, a nucleic acid template encoding both a scaffold protein and a POI, and optionally one or more phospholipids is provided. A nucleic acid template present in an in vitro protein synthesis system may also encode more than one type of POI and/or type of scaffold protein. Following translation of the nucleic acid template or templates, the scaffold proteins, POIs and phospholipids (when present) form a complex that enhances the solubility of the POI. The nucleic acid templates in an in vitro protein synthesis system may be bound to a solid support, such as, for example, a bead, matrix, chip, array, membrane, sheet, dish, or plate.

The in vitro protein synthesis system preferably includes at least one chemical energy source for providing the energy for protein synthesis. Non-limiting examples of energy sources are nucleotides, such as ATP or GTP, glycolytic intermediates, phosphorylated compounds, and energy-generating enzymes. In vitro protein synthesis systems described herein may further comprise free amino acids, tRNAs, labels, salts, buffering compounds, reducing agents enzymes, inhibitors, or cofactors.

In vitro protein synthesis systems of the invention can further comprise one or more detergents or surfactants or one or more lipids, such as but not limited to one or more phospholipids.

In some aspects of the present invention, an IVPS system can include a cell extract and nanoscale phospholipid bilayer discs in which the nanoscale phospholipid bilayer discs include components of the protein translocation machinery. Suitable components of the protein translocation machinery may include, for example, Sec YEG proteins or mammalian counterparts, the protein translocation (pore-forming) proteins, the SRP receptor, the ribosome receptor, and the like, in order to facilitate membrane protein insertion. Other proteins such SecA, SecB, or FtsY (among others) might be exogenously added to the reaction. Chaperonins that aid in protein folding and membrane insertion can also be added. POI components of the protein translocation machinery may be provided in pre-made PPPs, in which case the protein translocation proteins can be inserted through solubilization/dialysis methods of making PPPs, or may be inserted into PPPs using in vitro translation systems, as described herein.

Certain methods described herein improve the process for manufacturing PPPs. For instance, methods are provided wherein a detergent in included during the preparation of a scaffold protein-phospholipid complex. The method preferably comprises combining a phospholipid and a detergent to produce a stock solution; combining a scaffold protein with the stock solution to produce a phospholipid protein particle mixture; removing the detergent from the mixture; and, expressing the membrane POI from a nucleic acid in the presence of the phospholipid protein particle such that the membrane POI is incorporated into the particle. In certain embodiments, the detergent is an anionic detergent such as cholate.

Methods for preparing phospholipids protein particles comprising a scaffold protein, a POI, a ligand of the POI, and optionally also including one or more phospholipids, as well as compositions comprising the same, are also provided. The method comprises expressing the POI from a nucleic acid molecule using an in vitro translation system in the presence of a phospholipid, a scaffold protein, and the ligand. The phospholipids and scaffold protein may be complexed prior to expression of the POI (e.g., a form of PPP), or may associate during the in vitro translation process. These methods provide compositions comprising a POI, a scaffold protein, a ligand of the POI, and optionally one or more phospholipids. In certain embodiments, the ligand may include a detectable label. In others, association of the ligand with its POI causes the PPP (scaffold protein, phospholipids, POI and ligand) to become detectable by, for instance, inducing a detectable color change.

Also provided are compositions comprising one or more phospholipids, one or more scaffold proteins, one or more POIs, and/or one or more dyes. The dye is preferably a lipophilic dye such as DiR, DiI, DiD, and DiA. Such compositions may or may not include other detectable labels. Methods for visualizing or imaging such compositions, either in vitro or in vivo, are also described.

Also provided are compositions comprising a phospholipid, a scaffold protein, a POI, and a functional moiety such as a therapeutic or targeting agent. The therapeutic or targeting agent may be, for example, an antibody, peptide or ligand that directs the composition to a particular cell type or tissue in an in vitro or in vivo setting. Such compositions may or may not include dyes or other detectable labels. Also provided are methods for using such compositions to treat patients or visualize or image cells or tissues of a patient. These methods and compositions may also be used in in vitro assays.

In some embodiments of the invention, the methods further include isolating the POI from the in vitro synthesis mixture. Isolation can be, for example, by means of a peptide tag that is part of the POI, or by a peptide tag that is part of scaffold protein or is separately associated with the PPP. Labeled free amino acids, or labeled amino acid moieties of charged tRNAs may also be utilized. In embodiments that include synthesizing a POI in an in vitro synthesis system that includes phospholipid-protein particles, isolation can also be by means of an affinity tag that is attached to a lipid or lipid analog that is incorporated into the phospholipid-protein particle that is present in the in vitro protein synthesis mixture.

Kits are also provided. The kits preferably include a cell extract and at least one scaffold protein or at least one nucleic acid encoding a scaffold protein. The kit may optionally further include one or more of a solution of one or more amino acids, one or more buffers, one or more salts, one or more nucleotides, one or more enzymes, one or more inhibitors, one or more energy sources, one or more lipids, one or more phospholipids, one or more surfactants, one or more detergents, one or more nucleic acid vectors, or one or more nucleic acid constructs encoding, for example, a POI. The kit may include a cell extract and at least one PPP composition, which may be present in the cell extract, or may be provided separately. The scaffold protein may be present in the cell extract, or can be provided separately as a solid or in solution. The nucleic acid template may be an RNA construct or a DNA construct and can be provided as a solid, such as a lyophilate, or in solution. The kit may also optionally include instructions for use.

In certain embodiments, commercial services for performing a method and/or that uses a composition contemplated herein is provided. In one embodiment, one such service may include, without limitation, performing a drug screening method by, for example, contacting an isolated PPP comprising a target protein (e.g., a POI as described herein) with a test compound and detecting a change in the target protein. In another embodiment, the service may be a protein expression service, in which a POI is produced within a PPP comprising the protein. In illustrative embodiments, the protein is produced using in vitro translation.

The methods and compositions described herein are not limited to specific compositions or process steps, as such may vary. Features of particular embodiments may be combined with features of other disclosed embodiments of the invention, or with features of related technologies as they are known in the art, such as but not limited to, in vitro translation systems; protein engineering, protein, protein complex, and membrane protein isolation and structural analysis; protein and lipid labeling; protein assays (including but not limited to assays for membrane protein function, such as, for example, binding activity, signaling activity, kinase or other enzymatic activity, transporter activity, ion channel activity, etc.), including fluorescence-based assays; and the like as they are known in the art, to create further embodiments. Section headings provided herein are for convenience of the reader only, and are not intended to limit the scope of the invention.

### BRIEF DESCRIPTION OF THE DRAWINGS

FIG. 1 depicts a gel on which aliquots of whole (“W”) IVPS reactions or soluble fractions (“S”) of IVPS reactions were loaded. Bacteriorhodopsin was synthesized in an IVPS system that included PPPs made using Apolipoprotein A1 and phospholipid. Lanes 2 and 3 are aliquots of reactions that included 5 mg/mL PPPs made with a 70:1 ratio of DMPC to ApoA1; Lanes 4 and 5 are aliquots of reactions that included 5 mg/mL PPPs made with a 140:1 ratio of DMPC to ApoA1; and lanes 6 and 7 are aliquots of reactions that included 5 mg/mL PPPs made with a 140:1 ratio of DMPC to ApoA1. Lanes 13 and 14 are aliquots of reactions that included 5 mg/mL of Apo A1 protein but did not include PPPs.

FIG. 2 depicts a gel on which total (“T”) IVPS reactions or soluble fractions (“S”) of IVPS reactions were loaded. Bacteriorhodopsin was synthesized in the presence of 35S methionine label. Lanes 1 and 2 are reactions in the absence of MSP1. Lanes 3 and 4 are aliquots of reactions in which the MSP1 gene was added to the IVPS system. Lanes 5 and 6 are aliquots of reactions that included nucleic acid templates for both Bacteriorhodopsin and MSP1. Lanes 7 and 8 are aliquots of reactions that included both Bacteriorhodopsin and MSP1 nucleic acid templates, and also included phospholipid (DMPC, 30 ug). Lanes 9 and 10 include aliquots of control reactions that included pre-formed, purified PAPS that included MSP1 and DMPC).

FIG. 3 A) is a table of GPCR proteins that were translated in IVPS systems that contained or did not contain PPPs. B) is an autoradiographed gel showing electrophoresed samples of soluble (S) and total (T) protein synthesized in the absence (−) and presence (+) of PPPs for one GPCR protein (serotonin receptor HTR1; IOH46452). C) shows the total yields of several GPCR proteins synthesized in vitro in the presence of PPPs, and D) shows the percent solubility for IVPS reactions that included (black bars, on right) or did not include (gray bars, on left) PPPs in the IVPS reactions.

FIG. 4 A) is an autoradiogram of Ni-NTA column fractions of an incubated IVPS system in which GFP was synthesized in a rabbit reticulocyte extract that included PPPs and his-tagged MSP1. B) is an autoradiogram of Ni-NTA column fractions of an incubated IVPS system in which the adrenomedullin receptor was synthesized in a rabbit reticulocyte extract that included PPPs that included his-tagged MSP1. C) is an autoradiogram of Ni-NTA column fractions of an incubated IVPS system in which GFP was synthesized in a wheat germ extract that included PPPs that included his-tagged MSP1. D) is an autoradiogram of Ni-NTA column fractions of an incubated IVPS system in which the adrenomedullin receptor was synthesized in a wheat germ extract that included PPPs that included his-tagged MSP1. L, load, FT, flow through, W1, wash 1 W2 wash 2, W3 wash 3, E1, elution 1, E2, elution 2.

FIG. 5 shows PPPs labeling with Di dyes.

FIG. 6 shows the results of FRET experiments with A) Lumio-tagged EmrE-containing PPPs (no lipid label), B) DiI labeled PPPs (no EmrE present); and C) Lumio-tagged EmrE inserted into PPPs having incorporated DiI.

FIG. 7 shows the results of an EmrE ligand binding assay using Ni-NTA agarose beads.

FIG. 8 demonstrates affinity chromatography purification of EmrE-PPP.

### DETAILED DESCRIPTION

Unless defined otherwise, all technical and scientific terms used herein have the same meaning as commonly understood by one of ordinary skill in the art to which this invention is related. The following terms are defined for purposes of the invention as described herein. The singular form “a”, “an” and “the” include plural referents unless the context clearly dictates otherwise. Thus, for example, reference to “a ligand” includes a plurality of ligands and reference to “an antibody” includes a plurality of antibodies, etc.

As used herein, the terms “about” or “approximately” when referring to any numerical value are intended to mean a value of ±10% of the stated value. For example, “about 50° C.” (or “approximately 50° C.”) encompasses a range of temperatures from 45° C. to 55° C., inclusive. Similarly, “about 100 mM” (or “approximately 100 mM”) encompasses a range of concentrations from 90 mM to 110 mM, inclusive.

The terms “in vitro protein synthesis” (IVPS), “in vitro translation”, “cell-free translation”, “RNA template-driven in vitro protein synthesis”, “RNA template-driven cell-free protein synthesis” and “cell-free protein synthesis” are used interchangeably herein and are intended to refer to any method for cell-free synthesis of a protein. In vitro transcription-translation (IVTT) is one non-limiting example of IVPS.

The terms “in vitro transcription” and “cell-free transcription” are used interchangeably herein and are intended to refer to any method for cell-free synthesis of RNA from DNA without synthesis of protein from the RNA. A preferred RNA is messenger RNA (mRNA), which encodes proteins.

The terms “in vitro transcription-translation” (IVTT), “cell-free transcription-translation”, “DNA template-driven in vitro protein synthesis” and “DNA template-driven cell-free protein synthesis” are used interchangeably herein and are intended to refer to any method for cell-free synthesis of mRNA from DNA (transcription) and of protein from mRNA (translation).

As used herein, the term “gene” refers to a nucleic acid that encodes a polypeptide, protein, or untranslated RNA (e.g., rRNA, tRNA, anti-sense RNA). The gene can also include a promoter, as well as other sequences involved in expression of an RNA or protein.

As used herein, the phrase “nucleic acid molecule” refers to a sequence of contiguous nucleotides (riboNTPs, dNTPs, ddNTPs, or combinations thereof) of any length. A nucleic acid molecule may encode a full-length polypeptide or a fragment of any length thereof, or may be non-coding. As used herein, the terms “nucleic acid molecule” and “polynucleotide” may be used interchangeably and can refer to RNA, DNA, or synthetic nucleic acids (for example, peptide nucleic acid molecule, a nucleic acid molecule that includes sugar residues other than ribose or deoxyribose (e.g., a “locked” nucleic acid molecule), or a nucleic acid molecule that includes any combination of these. A nucleic acid molecule can include one or more non-naturally occurring bases, including derivatized bases.

“Operably linked” refers to a juxtaposition wherein the components so described are in a relationship permitting them to function in their intended manner. For example, a control sequence operably linked to a coding sequence is positioned in such a way that expression of the coding sequence is achieved under conditions compatible with control sequences.

As used herein, the term “polypeptide” refers to a sequence of contiguous amino acids of any length. The terms “peptide,” “oligopeptide,” or “protein” may be used interchangeably herein with the term “polypeptide.”

A “mutation” is a change in the genome with respect to the standard wild-type sequence. Mutations can be deletions, insertions, or rearrangements of nucleic acid sequences at a position in the genome, or they can be single base changes at a position in the genome, referred to as “point mutations”.

A “substitution,” as used herein, refers to the replacement of one or more amino acids or nucleotides by different amino acids or nucleotides, respectively.

A “variant” of a polypeptide or protein, as used herein, refers to an amino acid sequence that is altered with respect to the referenced polypeptide or protein by one or more amino acids. Preferably a variant of a polypeptide retains at least one activity of the polypeptide. Preferably a variant of a polypeptide has at least 60% identity to the referenced protein over a sequence of at least 15 amino acids. More preferably a variant of a polypeptide is at least 70% identical to the referenced protein over a sequence of at least 15 amino acids. Protein variants can be, for example, at least 80%, at least 90%, at least 95%, or at least 99% identical to referenced polypeptide over a sequence of at least 15 amino acids. Protein variants of the invention can be, for example, at least 80%, at least 90%, at least 95%, or at least 99% identical to referenced polypeptide over a sequence of at least 20 amino acids. The variant may have “conservative” changes, wherein a substituted amino acid has similar structural or chemical properties (e.g., replacement of leucine with isoleucine). A variant may also have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, DNASTAR software.

“Conservative amino acid substitutions” are those substitutions that are predicted to least interfere with the properties of the original protein, i.e., the structure and especially the function of the protein is conserved and not significantly changed by such substitutions. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Conservative substitutions include: the exchange of one negatively charged amino acid for another, where negatively charged amino acids may include aspartic acid and glutamic acid; the exchange of one positively charged amino acid for another, where one positively charged amino acids include lysine and arginine; and the exchange of amino acids with uncharged polar head groups having similar hydrophilicity values, where one group of amino acids with similar hydrophobicity may include leucine, isoleucine, and valine, another group may include glycine and alanine, a third group may include asparagine and glutamine, a fourth group may include serine and threonine, and a fifth group may include phenylalanine and tyrosine. In another sense, conservative amino acids can include the substitution of any noncharged amino acid for any other noncharged amino acid, an aromatic amino acid for any other aromatic amino acid, a polar amino acid for any other polar amino acid, a noncharged and nonpolar amino acid for any other noncharged and nonpolar amino acid, an acidic amino acid for any other acidic amino acid, or a basic amino acid for any other basic amino acid.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, biotinylation, or any similar process that retains at least one biological or immunological function of the polypeptide from which it was derived.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 10, at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) or a later version, such as Version 2.2.12 released Aujust 28, 2005; 2.2.13 released Dec. 6, 2005, or 2.2.14, released May 7, 2006, with blastp set at default parameters. Such default parameters may be, for example: Matrix: BLOSUM62; Open Gap: 11 and Extension Gap: 1 penalties; Gap x drop-off. 50; Expect: 10; Word Size: 3 Filter: on.

“Substantially purified” refers to the state of a species or activity that is the predominant species or activity present (for example on a molar basis it is more abundant than any other individual species or activities in the composition) and preferably a substantially purified fraction is a composition wherein the object species or activity comprises at least about 50 percent (on a molar, weight or activity basis) of all macromolecules or activities present. Generally, a substantially pure composition will comprise more than about 80 percent of all macromolecular species or activities present in a composition, more preferably more than about 85%, 90%, or 95%.

The terms “detectably labeled” and “labeled” are used interchangeably herein and are intended to refer to situations in which a molecule (e.g., a nucleic acid molecule, protein, nucleotide, amino acid, and the like) have been tagged with another moiety or molecule that produces a signal capable of being detected by any number of detection methods, such as by instrumentation, eye, photography, radiography, and the like. In such situations, molecules can be tagged (or “labeled”) with the molecule or moiety producing the signal (the “label” or “detectable label”) by any number of art-known methods, including covalent or ionic coupling, aggregation, affinity coupling (including, e.g., using primary and/or secondary antibodies, either or both of which may comprise a detectable label), and the like. Suitable detectable labels for use in preparing labeled or detectably labeled molecules in accordance with the invention include, for example, heavy isotope labels, heavy atom labels, radioactive isotope labels, fluorescent labels, chemiluminescent labels, bioluminescent labels and enzyme labels, and others that will be familiar to those of ordinary skill in the art.

The term “label” as used herein refers to a chemical moiety or protein that is directly or indirectly detectable (e.g. due to its spectral properties, conformation or activity) when attached to a target or compound and used in the present methods. The label can be directly detectable (fluorophore) or indirectly detectable (hapten or enzyme). Such labels include, but are not limited to, radiolabels that can be measured with radiation-counting devices; pigments, dyes or other chromogens that can be visually observed, imaged, or measured with a spectrophotometer; spin labels that can be measured with a spin label analyzer; heavy atom labels used, for example, in X-ray crystallography and NMR; heavy isotope labels used, for example, in mass spectrometry; and fluorescent labels (fluorophores), where the output signal is generated by the excitation of a suitable molecular adduct and that can be visualized by excitation with light that is absorbed by the dye or can be measured with standard fluorometers or imaging systems, for example. The label can be a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. In the context of the present invention, the term “label” typically does not include naturally occurring amino acids, such as amino acids that might be weakly fluorescent (e.g., tryptophan) or absorb in the UV. Such amino acids are not intended to be encompassed by the term “label” or “detectable label”. The term label can also refer to a “tag” or hapten that can bind selectively to a conjugated molecule such that the conjugated molecule, when added subsequently along with a substrate, is used to generate a detectable signal. For example, one can use biotin as a tag and then use an avidin or streptavidin conjugate of horseradish peroxidate (HRP) to bind to the tag, and then use a calorimetric substrate (e.g., tetramethylbenzidine (TMB)) or a fluorogenic substrate such as Amplex Red reagent (Molecular Probes, Inc.) to detect the presence of HRP. Numerous labels are know by those of skill in the art and include, but are not limited to, particles, fluorophores, haptens, enzymes and their calorimetric, fluorogenic and chemiluminescent substrates and other labels that are described in RICHARD P. HAUGLAND, MOLECULAR PROBES HANDBOOK OF FLUORESCENT PROBES AND RESEARCH PRODUCTS (9^{th} edition, CD-ROM, September 2002), supra.

A “tag” or an “amino acid sequence tag” is a series of amino acids that can be specifically bound by an affinity reagent. Examples of tags that can be incorporated into proteins for capture or detection of the protein using an affinity reagent include, without limitation, his tags comprising multiple (four or more, typically six) histidines, FLAG tag, Hemaglutinin tag, myc tag, or amino acid sequences derived from: glutathione-S-transferase, maltose binding protein, calmodulin, chitin binding protein, etc. Another amino acid sequence tag is a tetracysteine-containing lumio tag that can be used for purification or detection of a protein using a tetraaresenical or biarsenical reagent (see, e.g., U.S. Pat. Nos. 6,054,271; 6,008,378; 5,932,474; 6,451,569; WO 99/21013, which are incorporated into the present disclosure by reference).

A “solid support” is a solid material having a surface for attachment of molecules, compounds, cells, or other entities. A solid support can be a chip or array that comprises a surface, and that may comprise glass, silicon, nylon, polymers, plastics, ceramics, or metals. A solid support can also be a sheet of material, such as a membrane, such as a paper or other fiber, nylon, nitrocellulose, or polymeric sheet or membrane, or a plate or dish and can be comprised of glass, ceramics, metals, or plastics, such as, for example, a 96-well plate made of, for example, polystyrene, polypropylene, polycarbonate, or polyallomer. A solid support can also be a bead or particle of any shape, and is preferably spherical or nearly spherical, and preferably a bead or particle has a diameter or maximum width of 1 millimeter or less, more preferably of between 0.1 to 100 microns. Such particles or beads can be comprised of any suitable material, such as glass or ceramics, and/or one or more polymers, such as, for example, nylon, TEFLON™ polymer (polytetrafluoroethylene), polystyrene, polyacrylamide, sepaharose, agarose, cellulose, cellulose derivatives, or dextran, and/or can comprise metals, particularly paramagnetic metals, such as iron.

As used herein “associated with” means directly or indirectly bound to. A first biomolecule that is associated with s second biomolecule can be co-isolated with the second biomolecule using at least one capture or separation procedure that is based on the binding or mobility properties of the second biomolecule.

A “phosphophospholipid-protein particle” (“PPP”) is a molecular complex that includes at least one protein bound to at least one phospholipid. The protein is preferably a scaffold protein that includes at least one amphipathic alpha helix, and preferably is bound to a plurality of phospholipid molecules that are arranged in a bilayer. For example, a PPP based on apolipoprotein fragments that have amphipathic helical structures is described in Vanloo et al. (1995) Journal of Lipid Research 36: 1686-1696. A phosphophospholipid-protein particle is preferably in a discoidal shape of nanometer dimensions (e.g., from about 1 nm to about 995 nanometers in diameter, or more typically, from about 2 to about 700 nm in diameter, or from about 4 to about 600 nanometers in diameter, or from about 4 to about 400 nanometers in diameter, or from about 4 to about 200 nanometers in diameter, or from about 4 to about 100 nanometers in diameter, or from about 4 to about 50 nanometers in diameter, or from about 4 to about 20 nanometers in diameter. Where a protein bound to the phospholipid of a PPP is a naturally-occurring apolipoprotein, a variant of a naturally-occurring apolipoprotein, or an engineered apolipoprotein, a PPP may also be referred to as “phosphophospholipid-apolipoprotein particle” (PAP). PPPs may also be also referred to as or “Nanoscale Lipid Particles” (NLPs), or where the PPPs include any of the membrane scaffold proteins described in U.S. Patent Application Publication 2005/0182243, the PPPs may be referred to as “nanodiscs”. PPPs may also include other proteins such as a protein of interest (POI).

A “phosphophospholipid-apolipoprotein particle” (“PAP”) is a molecular complex that includes at least one apolipoprotein and at least one phospholipid, in which the phospholipid is arranged in a bilayer, and typically in a discoidal shape of nanometer dimensions (e.g., from about 1 nm to about 995 nanometers in diameter, or more typically, from about 2 to about 700 nm in diameter, or from about 4 to about 600 nanometers in diameter, or from about 4 to about 200 nanometers in diameter, or from about 4 to about 100 nanometers in diameter, or from about 4 to about 50 nanometers in diameter, or from about 4 to about 20 nanometers in diameter. Naturally-occurring and synthetic phophophospholipid-apolipoprotein particles are described, for example, in Pownall et al. (1978) Biochemistry 17: 1183-1188; Pownall et al. (1981) Biochemistry 20: 6630-6635; Jonas et al. (1984) J. Biol. Chem. 259: 6369-6375; Jonas et al. (1989) J. Biol. Chem. 264: 4818-4824; Jonas et al. (1993) J. Biol. Chem. 268: 1596-1602; Leroy et al. (1993) J. Biol. Chem. 268: 4798-4805; Tricerri et al. (2000) Biochemistry 39: 14682-14691; Segall et al. (2002) J. Lipid Res. 43: 1688-1700; Manchekar et al. (2004) J. Biol. Chem. 279: 39757-39766; Pearson et al. (2005) J. Biol. Chem. 280: 38576-38582, all incorporated by reference herein in their entireties.

The term “FRET” means fluorescence resonance energy transfer, and refers to the radiationless transmission of an energy quantum from its site of absorption to the site of its utilization in a molecule, or system of molecules, by resonance interaction between fluorophores, over distances considerably greater than interatomic, without substantial conversion to thermal energy, and without the donor and acceptor coming into kinetic collision. Fluorescence time-resolved fluorescence resonance energy transfer (TRET) is one type of FRET.

A “FRET donor” or “donor” is a moiety that initially absorbs energy (e.g., optical energy), and a “FRET acceptor” or “acceptor” is the moiety to which the energy is subsequently transferred. Nonlimiting examples of acceptors include coumarins and related fluorophores; xanthenes such as fluoresceins; fluorescent proteins; rhodols, and rhodamines; resorufins; cyanines; difluoroboradiazaindacenes; and phthalocyanines. Together the donor and acceptor form a “FRET pair” that operates via resonance energy transfer.

In FRET applications, acceptors may re-emit energy transferred from a donor fluorescent moiety. In other FRET applications, acceptors generally do not re-emit the transferred energy and are sometimes referred to as “fluorescence quenchers.” A fluorescent donor moiety and a quenching acceptor moiety may be referred to herein as a “quenching FRET pair”, Examples of fluorescence quenchers include indigos; benzoquinones; anthraquinones; azo compounds; nitro compounds; indoanilines; and di- and triphenylmethanes.

The term “quencher” refers to a molecule or part of a compound that is capable of reducing light emission (e.g. fluorescence emission) from a detectable moiety. Such reduction includes reducing the emission of light after the time when a photon is normally emitted from a fluorescent moiety. Quenching may occur by any of several mechanisms, including resonance energy transfer (RET), fluorescence resonance energy transfer (FRET), photo-induced electron transfer, paramagnetic enhancement of intersystem crossing, Dexter exchange coupling, dark quenching, and excitation coupling (e.g., the formation of dark complexes). Preferred quenchers include those that operate by FRET.

Other terms used in the fields of recombinant nucleic acid technology, biochemistry, and molecular and cell biology as used herein will be generally understood by one of ordinary skill in the applicable arts.

### IVPS Systems

The invention uses in vitro protein synthesis systems such as those known in the art, which can include cell extracts of prokaryotic or eukaryotic cells. The cell extracts can be from cells that are mutated in one or more genes, such as, for example, nuclease-encoding genes or protease-encoding genes, or can be cells engineered to express or overexpress one or more endogenous or exogenous genes, such as, for example, genes encoding tRNAs, polymerases, enzyme inhibitors, etc. The cell extracts may be supplemented with proteins or other molecules that can prevent template degradation, enhance transcription or translation, etc.

Nonlimiting examples of in vitro protein synthesis (IVPS) systems that can be used in the methods and compositions of the invention include but are not limited to those described in, for example, U.S. Pat. No. 5,478,730, to Alakhov et al., entitled “Method of preparing polypeptides in cell-free translation system”; U.S. Pat. Nos. 5,665,563; 5,492,817; and 5,324,637, to Beckler et al., entitled “Coupled transcription and translation in eukaryotic cell-free extract”; U.S. Pat. No. 6,337,191 to Swartz et al., entitled “In vitro Protein Synthesis using Glycolytic Intermediates as an Energy Source”; U.S. Pat. No. 6,518,058 to Biryukov et al., “Method of preparing polypeptides in cell-free system and device for its realization”; U.S. Pat. No. 6,670,173, to Schels et al., entitled “Bioreaction module for biochemical reactions”; U.S. Pat. No. 6,783,957 to Biryukov et al., entitled “Method for synthesis of polypeptides in cell-free systems”; United States Patent Application 2002/0168706 to Chatterjee et al., published Nov. 14, 2002, entitled “Improved in vitro synthesis system”; U.S. Pat. No. 6,168,931 to Swartz et al., issued Jan. 8, 2002, entitled “In vitro macromolecule biosynthesis methods using exogenous amino acids and a novel ATP regeneration system”; U.S. Pat. No. 6,548,276 to Swartz et al., issued Apr. 15, 2003, entitled “Enhanced in vitro synthesis of active proteins containing disulfide bonds”; United States Patent Application 2004/0110135 to Nemetz et al., published Jun. 10, 2004, entitled “Method for producing linear DNA fragments for the in vitro expression of proteins”; United States Patent Application 2004/0209321 to Swartz et al., published Oct. 21, 2004, entitled “Methods of in vitro protein synthesis”; United States Patent Application 2004/0214292 to Motoda et al., published Oct. 28, 2004, entitled “Method of producing template DNA and method of producing protein in cell-free protein synthesis system using the same”; United States Patent Application 2004/0259081 to Watzele et al., published Dec. 23, 2004, entitled “Method for protein expression starting from stabilized linear short DNA in cell-free in vitro transcription/translation systems with exonuclease-containing lysates or in a cellular system containing exonucleases”; United States Patent Applications 2005/0009013, published Jan. 13, 2005, and 2005/0032078, published Feb. 10, 2005, both to Rothschild et al. and both entitled “Methods for the detection, analysis and isolation of nascent proteins”; United States Patent Application 2005/0032086 to Sakanyan et al., published Feb. 10, 2005, entitled “Methods of RNA and protein synthesis”; Published PCT patent application WO 00/55353 to Swartz et al., published Mar. 15, 2000, entitled “In vitro macromolecule biosynthesis methods using exogenous amino acids and a novel ATP regeneration system”. All of these patents and patent applications are hereby incorporated by reference in their entireties.

The preparation of cell extracts that support the synthesis of proteins in vitro from purified mRNA transcripts, or from mRNA transcribed from DNA during the in vitro synthesis reaction are well known in the art. To synthesize a protein under investigation, a translation extract is “programmed” with an mRNA corresponding to the gene and protein under investigation. The mRNA can be produced from DNA, or the mRNA can be added exogenously in purified form. The RNA can be prepared synthetically from cloned DNA using RNA polymerases in an in vitro reaction.

Both prokaryotic cells and eukaryotic cells can be used for protein and/or nucleic acid synthesis according to the invention (see, e.g., Pelham et al, European Journal of Biochemistry, 67: 247, 1976). Prokaryotic systems can be used for simultaneous or “coupled” transcription and translation. The cell extracts used for IVTT contain the components necessary both for transcription (to produce mRNA) and for translation (to synthesize protein) in a single system. In such a system, the input template nucleic acid molecule is DNA.

As demonstrated by the Examples provided herein, the cell-free extracts used in the methods can be prokaryotic or eukaryotic extracts. Eukaryotic in vitro protein synthesis (IVPS) extracts include without limitation rabbit reticulocyte lysates, wheat germ lysates, Drosophila embryo extracts, scallop lysates (Storch et al. J. Comparative Physiology B, 173:611-620, 2003), extracts from mouse brain (Campagnoni et al., J Neurochem. 28:589-596, 1977; Gilbert et al. J Neurochem. 23:811-818, 1974), and chick brain (Liu et al. Transactions of the Illinois State Academy of Science, Volume 68, 1975). A eukaryotic extract for IVPS can be an extract of cultured cells. Cultured cells can be of any type. As nonlimiting examples, HeLa, COS, or CHO cell extracts can be used for in vitro translation systems.

Cells that can be used for preparing cell-free extracts include but are not limited to yeast cells (e.g., Saccharomyces cerevisiae cells and Pichia pastoris cells); insect cells (e.g., Drosophila (e.g., Drosophila melanogaster), Spodoptera (e.g., Spodoptera frugiperda Sf9 and Sf21 cells) and Trichoplusa (e.g., High-Five cells); nematode cells (e.g., C. elegans cells); avian cells (e.g., QT6 cells, QT-35 cells); amphibian cells (e.g., Xenopus laevis cells); reptilian cells; and mammalian cells (e.g., NIH3T3, 293, CHO, COS, VERO, C127, BHK, Per-C6, Bowes melanoma and HeLa cells). Cells from insects, mammals (such as hamsters, mouse, rat, gerbil, porcine, bovine, monkey, and humans), for example, sometimes are utilized. These and other suitable host cells are available commercially, for example, from Invitrogen Corporation, (Carlsbad, Calif.), American Type Culture Collection (Manassas, Va.), and Agricultural Research Culture Collection (NRRL; Peoria, Ill.).

Prokaryotic extracts can be from any prokaryotic cells, including, without limitation, gram negative and gram positive bacteria, including Escherichia sp. (e.g., E. coli), Klebsiella sp., Streptomyces sp., Streptocococcus sp., Shigella sp., Staphylococcus sp., Erwinia sp., Klebsiella sp., Bacillus sp. (e.g., B. cereus, B. subtilis and B. megaterium), Serratia sp., Pseudomonas sp. (e.g., P. aeruginosa and P. syringae), Salmonella sp. (e.g., S. typhi and S. typhimurium), and Rhodobacter sp. Bacterial strains and serotypes suitable for the invention can include E. coli serotypes K, B, C, and W. A typical prokaryotic cell extract is made from E. coli strain K-12. Cell extracts can be made from bacterial strains mutated to lack a nuclease or protease activity, or to lack the activity of one or more proteins that can interfere with purification or detection of translated proteins (see U.S. Patent Publication No. US2005/0136449, incorporated by reference herein in its entirety).

Cell-free extracts often are prepared from cells capable of performing one or more post-translational modifications of interest. Post translational modifications include, but are not limited to, addition of a phosphoryl, alkyl (e.g., methyl), fatty acid (e.g., myristoyl or palmitoyl), isoprenyl, glycosyl (e.g., polysaccharide), acetyl or peptidyl (e.g., ubiquitin) moiety to a synthesized protein or peptide and proteolytic cleavage of a portion of the synthesized target protein or target peptide. A cell utilized for preparing a cell-free extract sometimes is deficient in one or more native components, such as components that reduce DNA or RNA stability or components that interfere with translation or detection of the target proteins or peptides, which are known to those skilled in the art. Such components sometimes are reduced in cells by deleting or otherwise inactivating one or more genes or transcripts that encode a component. In some embodiments, the cells produce reduced amounts, non-detectable amounts or none of one or more of the following components: an exonuclease or endonuclease (e.g., an RNase such as RNase E, F, H, P and/or T; a DNase such as DNase I and/or II; a Rec protein; exonucleaseIII; exonuclease lambda; exonucleaseVII; endonuclease s1), topoisomerase and/or a component that binds to arsenic-containing agent (e.g., SlyD), for example (e.g., U.S. Patent application Publication no. 20050136449, filed Oct. 1, 2004, entitled “Compositions and Methods for Synthesizing, Purifying, and Detecting Biomolecules”, incorporated by reference herein in its entirety). Cell extracts sometimes are prepared from cells that express one or more suppressor tRNAs, such as a suppressor tRNA capable of loading any one of the twenty naturally occurring amino acids or an unnatural amino acid.

Eukaryotic extracts, optionally with added enzymes, substrates, and/or cofactors, can be used for translating proteins with post-translational modifications. Enzymes, substrates and/or cofactors for post-translational modification can also be added to prokaryotic extracts for IVPS. Cell-free extracts can be made using detergent, which is added to cells or cell lysate prior to centrifuging the lysate to make extract, as described in US Patent Application Publication No. 2006/0110788 (serial application Ser. No. 11/240,651, incorporated by reference herein in its entirety), herein incorporated by reference in its entirety for all disclosure of methods and compositions for in vitro protein synthesis systems. For example, nonionic or zwitterionic detergents can be used in the preparation of translation extracts, at concentrations at or slightly above the CMC.

IVPS systems can allow simultaneous and rapid expression of various proteins in a multiplexed configuration, for example in an array format, and can be used for screening of multiple proteins. IVTT systems that use DNA templates can provide increased efficiency in these formats by eliminating the need to separately synthesize and subsequently purify RNA transcripts. In addition, various kinds of unnatural amino acids or labeled amino acids can be efficiently incorporated into proteins for specific purposes using IVPS systems (see, for example, Noren et al., Science 244:182-188, 1989, incorporated by reference herein in its entirety).

In certain aspects, the cellular extract or an IVPS system that uses the extract, additionally includes at least one other component of any of the components in U.S. Pub. Pat. App. No. 2002/0168706, incorporated herein in its entirety. For example, the cellular extract can include one inhibitor of at least one enzyme, e.g., an enzyme selected from the group consisting of a nuclease, a phosphatase and a polymerase; and optionally the extract can be modified from a native or wild type extract to exhibit reduced activity of at least one enzyme, e.g., an enzyme selected from the group consisting of a nuclease, a phosphatase and a polymerase; and at least two energy sources that supply energy for protein and/or nucleic acid synthesis. In certain aspects the extract includes the Gam protein.

Enzymes, substrates and/or cofactors for post-translational modification can optionally be added to prokaryotic or eukaryotic extracts for IVPS, or may be present in a eukaryotic cell extract.

In addition to a cell extract, an IVPS typically includes at least one amino acid that is added to the cell extract. Typically, an IVPS comprises a cell extract, at least one amino acid, and at least one added energy source that supports translation. Where the in vitro translation system is a transcription/translation system, a polymerase is also preferably added. Where the in vitro translation system is a transcription/translation system, a polymerase is also preferably added. In vitro protein synthesis systems, including their manufacture and methods of use, are well known in the art. In exemplary embodiments, at least two amino acids and at least one compound that provides energy for translation is added to a cell extract to provide an IVPS system. In some exemplary embodiments, an IVPS comprises a cell extract, the twenty naturally-occurring amino acids, and at least one compound that provides energy for translation. In some preferred embodiments, an IVPS includes at least two compounds that serve as energy sources for translation, at least one of which can be a glycolytic intermediate. At least one of the amino acids provided in an IVPS system can optionally be labeled, for example, one or more amino acids can be radiolabeled for detection of a translated protein that incorporates the labeled amino acid. In some embodiments, a feeding solution that comprises one or more additional energy sources and additional amino acids is added after an initial incubation of the IVPS. Feeding solutions for IVPS systems and their use are described in U.S. Patent Application Publication No. 2006/0110788, incorporated by reference herein.

Some examples of IVPS systems and other related embodiments are disclosed in U.S. Patent Application Publication No. 2002/0168706, “Improved In vitro Synthesis Systems” filed Mar. 7, 2002; U.S. Patent Application Publication No. 2005/0136449, “Compositions and Methods for Synthesizing, Purifying, and Detecting Biomolecules” filed Oct. 1, 2004; U.S. Patent Application Publication No. 2006/0084136, “Production of Fusion Proteins by Cell-Free Protein Synthesis” filed Jul. 14, 2005; U.S. Patent Application Publication No. 2006/0110788, “Feeding Buffers, Systems, and Methods for In vitro Synthesis” filed Oct. 1, 2005; U.S. Patent Application Publication No. 2006/0110788, “Feeding Buffers, Systems, and Methods for In vitro Synthesis” filed Oct. 1, 2005; and U.S. Patent Application Publication No. 2006/0211083, filed Jan. 20, 2006, “Products and Processes for In vitro Synthesis of Biomolecules” the disclosures of which applications are incorporated by reference herein in their entireties.

In some embodiments, the invention uses Invitrogen's EXPRESSWAY™ in vitro translation systems (Invitrogen, Carlsbad, Calif.) that include a cell-free S30 extract and a translation buffer. The S30 extract contains the majority of soluble translational components including initiation, elongation and termination factors, ribosomes and tRNAs from intact cells. The translation buffer contains amino acids, energy sources such as ATP and GTP, energy regenerating components such as phosphoenol pyruvate/pyruvate kinase, acetyl phosphate/acetate kinase or creatine phosphate/creatine kinase and a variety of other important co-factors (Zubay, Ann. Rev. Genet. 7:267-87, 1973; Pelham and Jackson, Eur J Biochem. 67:247, 1976; and Erickson and Blobel, Methods Enzymol. 96; 38-50, 1983). The reaction buffer, methionine, T7 Enzyme Mix, and DNA template of interest, operably linked to a T7 promoter, are mixed with the E. coli extract. As the DNA template is transcribed, the 5′ end of the mRNA becomes bound by ribosomes and undergoes translation to synthesis the encoded protein.

### Scaffold Proteins

Described herein are methods and compositions for using scaffold proteins such as AAHC proteins or apolipoproteins in an IVPS system. An apolipoprotein can be present in a cell extract when a template encoding a POI is added, or can be added during the synthesis reaction, or an apolipoprotein can be translated from a nucleic acid construct added to the IVPS system.

Apolipoproteins are proteins that bind and transport lipids in the circulatory system of animals. Sequence homology studies among different apolipoproteins and across species and structural analysis and predictions indicate that apolipoproteins have similar structures, which includes several amphipathic helices. Accordingly, variant apolipoproteins or engineered apolipoproteins provided herein typically include at least one and can include 2, 3, 4, or more amphipathic helices, and typically include the sequence of an amphipathic helix of a wild-type or naturally-occurring apolipoprotein, or a conservative amino acid substitution thereof. Furthermore, a variant or engineered apolipoprotein used in the methods and compositions of the invention typically retains the ability to bind lipids.

Apolipoprotein variants can be tested for the ability to bind lipid and to form particles, such as discoidal particles, by methods known in the art, such as but not limited to electron microscopy, scanning probe microscopy, atomic force microscopy, circular dichroism, infrared spectroscopy, fluorescence polarization measurements, and gel filtration (size fractionation). See, for example, Vanloo et al. (1995) Journal of Lipid Research, 36: 1686-1696, as well as U.S. Pat. No. 7,048,949; U.S. Pat. No. 7,083,958; and U.S. Patent Application Publication 20050182243; all of which are incorporated by reference in their entireties.

As used herein, the term “apolipoprotein” is used broadly to mean proteins that bind lipids, and are soluble in aqueous solution in both their free and lipid-bound forms. Apolipoproteins of the invention have at least one helical domain that preferably forms, or is predicted to form, an amphipathic helix. Apolipoproteins used in the methods and compositions of the invention preferably are either: naturally-occurring apolipoproteins, which can be of any species origin, sequence variants of naturally-occurring apolipoproteins, as described in more detail below, or engineered proteins having at least one helical domain that has at least 70% homology to at least 15 amino acids or at least 90% homology to at least 10 amino acids of at least one helical domain of a naturally-occurring apolipoprotein. Apolipoproteins used in the methods and compositions of the present invention have the property of, when present in an IVPS system (an in vitro translation system), increasing the soluble yield of a membrane protein by at least 10%, where the soluble yield is calculated as either: the amount of soluble protein synthesized, or the percentage of soluble protein to total protein synthesized.

In some embodiments, an apolipoprotein used in the methods and compositions of the invention can comprise the sequence of a non-truncated naturally-occurring mature, processed form of an apolipoprotein. In some embodiments, an apolipoprotein used in the methods and compositions of the invention can comprise the sequence of a non-truncated naturally-occurring “pro” form of an apolipoprotein, with an unprocessed N-terminus. In some embodiments, an apolipoprotein used in the methods and compositions of the invention can comprise the sequence of a non-truncated naturally-occurring precursor form of an apolipoprotein, with an unprocessed N-terminus, and at least a portion of the signal peptide. These apolipoprotein forms can include additional sequences, such as but not limited to amino acid tag sequences.

Apolipoproteins used in the methods and compositions of the invention include apolipoprotein variants, including proteins having at least 10, 15, 20, 25, 50, 75, 100, 150, or 200 consecutive amino acids that have at least 50, 60, 70, 75, 80, 85, 90, 95, 96, 97, 98, or 99% sequence identity to a wild-type apolipoprotein of any species, in which the variant, when present in an IVPS system, increases the solubility of at least one protein translated in the IVPS system by at least 10%. In certain aspects, the soluble protein produced in an IVPS system is increased by at least 15%, 20%, or 25%, or is increased in a detectable manner, over the same protein produced in the IVPS system in the absence of the apolipoprotein or variant thereof. Apolipoprotein variants can have one or more sequence deletions or insertions with respect to naturally-occurring apolipoproteins. As nonlimiting examples, amino acid tag sequences can be added, or non-helical domains deleted in some apolipoprotein variants.

A variant apolipoprotein, in certain aspects, is a variant of a wild-type mammalian apolipoprotein, especially a variant of Apolipoprotein A-I (Apo A-I), Apolipoprotein A-II (Apo A-II), Apolipoprotein A-IV (Apo A-IV), Apolipoprotein A-V (Apo A-V), Apolipoprotein B-100 (Apo B-100), Apolipoprotein B-48 (Apo B-48), Apolipoprotein C-I (Apo C-I), Apolipoprotein C-II (Apo C-II), Apolipoprotein C-III (Apo C-III), Apolipoprotein D (Apo D), Apolipoprotein E (Apo E), Apolipoprotein H (Apo H), or Lipoprotein (a) (Lp(a)).

Some apolipoproteins, called exchangeable apolipoproteins, reversibly bind lipid, and have stable conformations when bound to lipid and when not bound to lipid. The exchangeable apoplipoproteins are typically less than about 50 kDa in size, and share structural similarity based on a variable number of amphipathic alpha helical domains that are thought to bind the surface of lipoprotein particles (Segrest et al. J. Lipid Res. 33: 141-166 (1992); Pearson et al. J. Biol. Chem. 280, 38576-38582 (2005); Boguski et al. Proc. Natl. Acad. Sci. U.S.A. 83: 8457-8461 (1985)). The invention includes the use of exchangeable apolipoproteins and their variants in the methods and compositions of the invention. Exchangeable apolipoproteins include, without limitation, Apolipoprotein A-I, Apolipoprotein A-II, Apolipoprotein A-IV, Apolipoprotein A-V, Apolipoprotein C-I, Apolipoprotein C-II, Apolipoprotein C-III, Apolipoprotein E, and Apoliphorin III.

The apolipoproteins used in the compositions and methods of the invention can be of any animal origin, or based on the sequence of apolipoproteins of any animal species. In some embodiments, the apolipoprotein used in the method of the invention is a mammalian apolipoprotein, is an apolipoprotein variant that has one or more sequences derived from a sequence of one or more mammalian apolipoproteins, such as, for example, Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, or Lipoprotein (a). The designations of these apolipoproteins used herein may originate from their identification in one or more species; in many cases, the names designate human proteins. For example, the sequences of human apolipoproteins include, without limitation: gi 37499465 (human apolipoprotein A1, SEQ ID NO:1), human proapolipoprotein A1 (SEQ ID NO:2); human apolipoprotein A-II (gi 296633, SEQ ID NO:3), human apolipoprotein A-IV (gi 178759, SEQ ID NO:4); human apolipoprotein A-V (gi 60391728, SEQ ID NO:5), Apolipoprotein B-100, (gi 114014, SEQ ID NO:6); Apolipoprotein B-48 (gi 178732, SEQ ID NO:7); Apolipoprotein C-I (gi 30583123, SEQ ID NO:8); Apolipoprotein C-II (gi 37499469; SEQ ID NO:9); Apolipoprotein C-III (gi 521205, SEQ ID NO:10); Apolipoprotein D (gi5466584, SEQ ID NO:11; gi 1246096, SEQ ID NO:12); Apolipoprotein E (gi 178853, SEQ ID NO:13); Apolipoprotein H (gi 178857, SEQ ID NO:14); and Apolipoprotein Lp(a) (gi 5031885, SEQ ID NO:15), and their variants having at least 10, 15, 20, 25, 50, 75, 100, 150, or 200 consecutive amino acids that have at least 50, 60, 70, 75, 80, 85, 90, 95, 96, 97, 98, or 99% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, and SEQ ID NO:18 are apolipoproteins that are included in the methods and compositions of the invention.

The designations of Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, or Lipoprotein (a) however are used herein to also refer to analogues of these proteins in species other than homo sapiens (including but not limited to species of mammal, fish, bird, marsupial, reptile, amphibian, mollusk, or arthropod). The analogues of the proteins referenced herein by their assigned name for homo sapiens proteins are thus included as apolipoproteins of the invention. Such apolipoproteins and apolipoprotein variants of the invention from species other than homo sapiens may or may not have the same name in other species.

As nonlimiting examples, an Apolipoprotein A-I of any of: rat (gi 6978515), mouse (gi 2145141), golden hamster (gi 4063843), Atlantic salmon (gi 64356), zebrafish (gi 18858281; NM_{—}113128; SEQ ID NO: 89), duck (gi 627301), pufferfish (gi 57157761), orangutan (gi 23379768), chimpanzee (gi 23379764), gorilla (gi 23379766), pig (gi 47523850), baboon (gi 86653), rabbit (gi 71790), or sequence variants thereof, can be used. As nonlimiting examples, an Apolipoprotein A-II of any of: rat (gi 202948), mouse (gi 7304897), macaque (gi 38049), cow (gi 6225059), horse (gi 47115663), or sequence variants thereof, can be used. As nonlimiting examples, an Apolipoprotein A-IV of any of: rat (gi 8392909), mouse (gi 6680702), chicken (gi 45384392), baboon (gi 510276), pig (gi 47523830), chimpanzee (gi 601801), or sequence variants thereof, can be used. As nonlimiting examples, an Apolipoprotein A-V of any of: rat (gi 18034777), mouse (gi 31560003), cow (gi 76635264), or dog (gi 57086253), or sequence variants thereof, can be used.

As nonlimiting examples, an Apolipoprotein B of any of: rat (gi 61098031), chicken (gi 114013), rabbit (gi 114015), lemur (gi 31558958), pig (gi 951375), macaque (gi 930126), squirrel (gi 31558956), hedgehog (gi 31558952), or sequence variants thereof, can be used.

As nonlimiting examples, an Apolipoprotein C-I of any of: rat (gi 6978521), mouse (gi 6680704), macaque (gi 114017), rabbit (gi 416626), or sequence variants thereof, can be used. As nonlimiting examples, an Apolipoprotein C-II of any of: mouse (gi 6753100), dog (gi 50979236), macaque (gi 342077), guinea pig (gi 191239), cow (gi 114019), pufferfish (gi 74096407), or sequence variants thereof, can be used. As nonlimiting examples, an Apolipoprotein C-III of any of: rat (gi 8392912), mouse (gi 15421856), dog (gi 50979230), pig (gi 50657386), cow (gi 47564119), or sequence variants thereof, can be used.

As nonlimiting examples, an Apolipoprotein D of any of: rat (gi 287650), mouse (gi 75677437), chicken (gi 58696426), guinea pig (gi 1110553), or deer (gi 82469911), or sequence variants thereof, can be used.

As nonlimiting examples, an Apolipoprotein E of any of: rat (gi 20301954), mouse (gi 6753102), chimpanzee (gi 57113897), rhesus monkey (gi 3913070), baboon (gi 176569), pig (gi 311233), cow (gi 312893), or sequence variants thereof, can be used.

As nonlimiting examples, an Apolipoprotein H of any of: rat (gi 56971279), mouse (gi 94400779), woodchuck (gi 92111519), dog (gi 54792721), cow (gi 27806741), or sequence variants thereof, can be used.

In some embodiments, an apolipoprotein used in the method of the invention is an insect apolipoprotein, or has sequences derived from the sequences of an insect apolipoprotein, such as, for example, Apoliphorin I, Apoliphorin II, or Apoliphorin III. Such proteins can be of any species, such as for example, Drospophila species, Manduca species, Locusta species, Lethocerus species, Ostrinia species, Bombyx species, and also their analogues in other insect or in non-insect species. For example, Apolipophorin I (gi 2498144, SEQ ID NO:16), Apolipophorin II (gi 2746729, SEQ ID NO:17); Apolipophorin III (gi 159481, SEQ ID NO:18), and apolipoprotein variants having at least 10, 15, 20, 25, 50, 75, 100, 150, or 200 consecutive amino acids that have at least 50, 60, 70, 75, 80, 85, 90, 95, 96, 97, 98, or 99% sequence identity to SEQ ID NO: 16, SEQ ID NO: 17, and SEQ ID NO: 18 are nonlimiting examples of apolipoproteins that can be used in the compositions and methods of the invention.

Apolipoproteins that can be present in an IVPS system of the invention include, without limitation, Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, Lipoprotein (a), Apoliphorin I, Apoliphorin II, or Apoliphorin III analogues of any species, including variants of analogues of any species.

In some exemplary embodiments, an apolipoprotein present in an IVPS system is an exchangeable apolipoprotein, such as, for example, Apolipoprotein A-I, Apolipoprotein A-II, Apolipoprotein A-IV, Apolipoprotein A-V, Apolipoprotein C-I, Apolipoprotein C-II, Apolipoprotein C-III, Apolipoprotein E, or Apoliphorin III.

In some embodiments, an apolipoprotein used in the compositions and methods of the invention has at least 70% identity to at least 20 consecutive or contiguous amino acids of an apolipoprotein, such as but not limited to, Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, Lipoprotein (a), Apoliphorin I, Apoliphorin II, or Apoliphorin III of any species. An apolipoprotein used in the methods and compositions of the invention has, in preferred embodiments, at least 70% identity to an apolipoprotein over a continuous sequence of at least 10 amino acids, 15 amino acids, least 20 amino acids, at least 30 amino acids, at least 40 amino acids, at least 50 amino acids, at least 60 amino acids, at least 70 amino acids, at least 80 amino acids, at least 90 amino acids, or at least 100 amino acids of the apolipoprotein. In some preferred embodiments, an apolipoprotein when present in an IVPS system improves the solubility of at least one protein synthesized in the IVPS system, and has at least 70% identity to an apolipoprotein over a continuous sequence of at least 10 amino acids, at least 15 amino acids, at least 20 amino acids, at least 30 amino acids, at least 40 amino acids, at least 50 amino acids, at least 60 amino acids, at least 70 amino acids, at least 80 amino acids, at least 90 amino acids, or at least 100 amino acids of the apolipoprotein. In some embodiments, an apolipoprotein used in the methods and compositions of the invention when present in an IVPS system improves the solubility of at least one protein synthesized in the IVPS system, and has at least 70%, at least 80%, at least 90%, at least 95%, or at least 99% identity to an apolipoprotein of any species over a continuous sequence of at least 20 amino acids.

In some embodiments, an apolipoprotein used in the compositions and methods of the invention has at least 70% at least 80%, at least 90%, at least 95%, or at least 99% identity to an exchangeable apolipoprotein, such as but not limited to, Apolipoprotein A-I, Apolipoprotein A-II, Apolipoprotein A-IV, Apolipoprotein C-I, Apolipoprotein C-II, Apolipoprotein C-III, Apolipoprotein E, or Apoliphorin III of any species over a continuous sequence of at least 10 amino acids, at least 15 amino acids, at least 20 amino acids, at least 30 amino acids, at least 40 amino acids, at least 50 amino acids, at least 60 amino acids, at least 70 amino acids, at least 80 amino acids, or at least 100 amino acids. In some embodiments, an apolipoprotein used in the methods and compositions of the invention when present in an IVPS system improves the solubility of at least one protein synthesized in the IVPS system, and has at least 70% identity to an apolipoprotein of any species over a continuous sequence of at least 10 amino acids, at least 15 amino acids, at least 20 amino acids, at least 30 amino acids, at least 40 amino acids, at least 50 amino acids, at least 60 amino acids, at least 70 amino acids, at least 80 amino acids, or at least 100 amino acids.

In some embodiments, an apolipoprotein is a mammalian apolipoprotein or has at least 70%, at least 80%, at least 90%, at least 95%, or at least 99% identity to a mammalian apolipoprotein such as, but not limited to, Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, or Lipoprotein (a) over a continuous sequence of at least 10 amino acids, at least 15 amino acids, at least 20 amino acids, at least 30 amino acids, at least 40 amino acids, at least 50 amino acids, at least 60 amino acids, at least 70 amino acids, at least 80 amino acids, or at least 100 amino acids.

In some embodiments, an apolipoprotein is an insect apolipoprotein such as Apoliphorin I, Apoliphorin II, or Apoliphorin III, or has at least 70%, at least 80%, at least 90%, at least 95%, or at least 99% identity to an insect Apoliphorin I, Apoliphorin II, or Apoliphorin III over a continuous sequence of at least 10 amino acids, at least 15 amino acids, at least 20 amino acids, at least 30 amino acids, at least 40 amino acids, at least 50 amino acids, at least 60 amino acids, at least 70 amino acids, at least 80 amino acids, or at least 100 amino acids.

In some exemplary embodiments, an apolipoprotein used in the methods and compositions of the invention is a wild-type exchangeable apolipoprotein or a variant thereof having at least 90% sequence identity to at least 100 contiguous amino acids of the wild-type exchangeable apolipoprotein, and capable of increasing the soluble protein production of a POI in an IVPS reaction by at least 10%. In some embodiments, an apolipoprotein used in the methods and compositions of the invention is Apolipoprotein A-I, Apolipoprotein A-II, Apolipoprotein A-IV, Apolipoprotein A-V, Apolipoprotein C-I, Apolipoprotein C-II, Apolipoprotein C-III, Apolipoprotein E, or Apoliphorin III, or a variant of any of these having at least 90% sequence identity to at least 100 contiguous amino acids of the wild-type exchangeable apolipoprotein, and capable of increasing the soluble protein production of a POI such as bacterial EmrE protein or a human GABA protein in an IVPS reaction by at least 10%.

In an exemplary embodiment, an apolipoprotein used in the methods and compositions of the invention is Apolipoprotein A-I or a variant of Apolipoprotein A-I having at least 90% sequence identity to at least 100 contiguous amino acids of wild-type Apolipoprotein A-I, and having the ability to increase soluble protein production of a POI by at least 10%.

Suitable apolipoproteins also include engineered apolipoproteins having at least 90% amino acid sequence identity with at least 10 residues or at least 15 residues of a helical domain of a naturally-occurring apolipoprotein. Such proteins include engineered apolipoproteins disclosed in US Patent Application Publication 2005/0182243, incorporated herein by reference in its entirety, such as histidine tagged MSP1 (SEQ ID NO: 19); MSP1 (SEQ ID NO:20); MSP2 (his tagged) (SEQ ID NO:21); MSP2 (his tagged, long linker) (SEQ ID NO:22); MSP1D5D6 (SEQ ID NO:23); MSP1D6D7 (SEQ ID NO:24); MAP1T4 (SEQ ID NO:25); MSP1T5 (SEQ ID NO:26); MSP1T6 (SEQ ID NO:27); MSP1N1 (SEQ ID NO:28); MSP1E3TEV (SEQ ID NO:29); MSP1E3D1 (SEQ ID NO:30); HisTEV-MSP2 (SEQ ID NO:31); MSP2N1 (SEQ ID NO:32); MSP2N2 (SEQ ID NO:33); MSP2N3 (SEQ ID NO:34); MSP2N4 (SEQ ID NO:35); MSP2N5 (SEQ ID NO:36); MSP2N6 (SEQ ID NO:37); MSP2CPR (SEQ ID NO:38); His-TEV-MSP1T2-GT (SEQ ID NO:39); MSP1RC12′ (SEQ ID NO:40); MSP1K90C (SEQ ID NO:41); and MSP1K152C (SEQ ID NO:42).

The apoplipoproteins used here may be from any source, for example, isolated from organisms or tissue, including blood, plasma, or serum, isolated from cell culture, or expressed recombinantly prior to be added to the in vitro synthesis system. Preferably, an apolipoprotein is at least partially purified prior its addition to an in vitro synthesis system.

The amino acid sequence of an apolipoprotein used in the methods and compositions of the invention can be modified with respect to the sequence of a wild-type apolipoprotein, having one or more deletions, additional amino acids, or amino acid substitutions with respect to a wild-type sequence, while having the property of enhancing the yield of protein in soluble form made in an IVPS reaction when the apolipoprotein is present in the IVPS reaction.

For example, an apolipoprotein used in the methods or compositions of the invention can have an N-terminal or C-terminal truncation, or can have one or more internal deletions or insertions with respect to a wild-type apolipoprotein sequence. An apolipoprotein used in the methods and compositions of the invention can be a multimer of an apolipoprotein or a portion thereof, for example, two or more copies of an apolipoprotein, or a variant or portion thereof, joined by a linker. An apolipoprotein used in the methods and compositions of the invention can be a chimeric apolipoprotein, comprising sequences of two different apolipoproteins (or variants thereof). Furthermore, the apolipoprotein can be bound to a peptide or another protein sequence, as part of a fusion protein. The peptide sequence can be a purification and/or detection tag, for example.

In some embodiments of the invention, apolipoproteins used in an IVPS include membrane scaffold proteins (MSPs) based on the sequence of Apolipoprotein A-1 disclosed in U.S. Pat. No. 7,048,949; U.S. Pat. No. 7,083,958; U.S. Patent Application Publication No. 2005/0182243 A1, 2005/0152984 A1, 2004/0053384 A1, and 2006/0088524 A1, all incorporated by reference herein in their entireties.

The apolipoprotein provided herein can be bound to a lipid or can be a lipid free apolipoprotein. For example, an apolipoprotein can be isolated from an organism (such as from blood or plasma), from tissue culture cells or media, or from bacterial cells engineered to express a recombinant apolipoprotein. An apolipoprotein can also by synthesized, for example, using chemical synthesis of peptides, optionally with peptide ligation to form larger peptides or proteins. The isolated apolipoprotein can be bound to lipid using methods known in the art (see, for example, Pownall et al. (1978) Biochemistry 17: 1183-1188; Pownall et al. (1981) Biochemistry 20: 6630-6635; Jonas et al. (1984) J. Biol. Chem. 259: 6369-6375; Jonas et al. (1989) J. Biol. Chem. 264: 4818-4824; Jonas et al. (1993) J. Biol. Chem. 268: 1596-1602; Tricerri et al. (2000) Biochemistry 39: 14682-14691; Segall et al. (2002) J. Lipid Res. 43: 1688-1700; Pearson et al. (2005) J. Biol. Chem. 280: 38576-38582, all incorporated by reference herein in their entireties). In some embodiments, apolipoproteins can be provided in IVPS systems that also include one or more naturally occurring or synthetic lipids such as but not limited to one or more phospholipids. Cholesterol, a cholesterol ester, or one or more other neutral lipids, such as, but not limited to, a sterol ester, a mono-, di-, or triacylglyceride, or an acylglycerol, can optionally also be included. Lipids can be present at a concentration of from about 1 microgram per milliliter to about 20 milligrams per milliliter, or from about 5 micrograms per milliliter to about 10 milligrams per milliliter, or from about 10 micrograms per milliliter to about 5 milligrams per milliliter. One or more phospholipids can be bound to an apolipoprotein in the IVPS system. In some embodiments of the invention, apolipoproteins are translated using in vitro protein systems that include one or more lipids, such as but not limited to one or more phospholipids. The apolipoproteins synthesized in the cell-free system can bind one or more lipids during or following translation.

Suitable scaffold proteins also include proteins with at least one amphipathic alpha helix, or that are predicted by amino acid sequence analysis to have at least one amphipathic alpha helix (amphipathic alpha helix containing proteins, or AAHC proteins), which may include an apolipoprotein described herein. These may also be used in the IVPS methods and compositions described herein. Such proteins preferably bind lipid, as can be demonstrated using art-recognized methods, including, but not limited to electron microscopy, scanning probe microscopy, atomic force microscopy, circular dichroism, infrared spectroscopy, fluorescence polarization measurements, and gel filtration (size fractionation). Nonlimiting examples of such AAHC proteins are apomyoglobin, synucleins (for example, synuclein alpha (SEQ ID NO:84), synuclein alpha (SEQ ID NO:85), synuclein beta (SEQ ID NO:86), synuclein beta (SEQ ID NO:87), synuclein gamma (SEQ ID NO:88), or peptabiols such as, for example, melitin, almethicin, or a gramicidin (such as gramicidin A, B, or C). Other examples of proteins that have one or more amphipathic helices can be found, for example, in Advances in Protein Chemistry, Volume 45, pages 303-369, Schumaker, ed., Academic Press, New York (1994), incorporated herein by reference in its entirety. Included in the compositions and methods of the invention are proteins that include sequences of naturally-occurring AAHC protein with at least 10, 15, 20, 25, 50, 75, 100, 150, or 200 consecutive amino acids that have at least 50, 60, 70, 75, 80, 85, 90, 95, 96, 97, 98, or 99% sequence identity to a wild-type or naturally-occurring AAHC protein of any species, in which the variants, when present in an IVPS system, increase the solubility of at least one protein translated in the IVPS system by at least 10%. In certain aspects, the soluble protein produced in an IVPS system is increased by at least 15%, 20%, or 25%, or is increased, optionally in a detectable manner, over the same protein produced in the IVPS system in the absence of the AAHC protein or variant thereof. AAHC protein variants can have one or more sequence deletions or insertions with respect to naturally-occurring AAHC proteins. As nonlimiting examples, amino acid tag sequences can be added, or non-helical domains deleted in some AAHC protein variants.

An AAHC protein used in the methods and compositions of the invention has, in preferred embodiments, at least 70% identity to an AAHC protein over a continuous sequence of at least 10 amino acids, over a continuous sequence of at least 15 amino acids, over a continuous sequence of at least 20 amino acids, over a continuous sequence of at least 30 amino acids, over a continuous sequence of at least 40 amino acids, over a continuous sequence of at least 50 amino acids, over a continuous sequence of at least 60 amino acids, over a continuous sequence of at least 70 amino acids, over a continuous sequence of at least 80 amino acids, over a continuous sequence of at least 90 amino acids, or over a continuous sequence of at least 100 amino acids of the AAHC protein. In some preferred embodiments, an AAHC protein when present in an IVPS system improves the solubility of at least one protein synthesized in the IVPS system, and has at least 70% identity to an apolipoprotein over a continuous sequence of at least 10 amino acids, over a continuous sequence of at least 15 amino acids, over a continuous sequence of at least 20 amino acids, over a continuous sequence of at least 30 amino acids, over a continuous sequence of at least 40 amino acids, over a continuous sequence of at least 50 amino acids, over a continuous sequence of at least 60 amino acids, over a continuous sequence of at least 70 amino acids, over a continuous sequence of at least 80 amino acids, over a continuous sequence of at least 90 amino acids, or over a continuous sequence of at least 100 amino acids of the AAHC proteins. In some embodiments, an AAHC protein used in the methods and compositions of the invention when present in an IVPS system improves the solubility of at least one protein synthesized in the IVPS system, and has at least 70%, at least 80%, at least 90%, at least 95%, or at least 99% identity to an AAHC protein of any species over a continuous sequence of at least 20 amino acids.

In some embodiments, an AAHC protein used in the compositions and methods of the invention has at least 70% at least 80%, at least 90%, at least 95%, or at least 99% identity to a peptabiol, a synuclein such as synuclein alpha (SEQ ID NO:84), synuclein alpha (SEQ ID NO:85), synuclein beta (SEQ ID NO:86), synuclein beta (SEQ ID NO:87), synuclein gamma (SEQ ID NO:88), or an apomyoglobin of any species over a continuous sequence of at least 10 amino acids, at least 15 amino acids, at least 20 amino acids, at least 30 amino acids, at least 40 amino acids, at least 50 amino acids, at least 60 amino acids, at least 70 amino acids, at least 80 amino acids, or at least 100 amino acids. In some embodiments, an AAHC protein used in the methods and compositions of the invention when present in an IVPS system improves the solubility of at least one protein synthesized in the IVPS system, and has at least 70% identity to an AAHC protein of any species over a continuous sequence of at least 10 amino acids, at least 15 amino acids, at least 20 amino acids, at least 30 amino acids, at least 40 amino acids, at least 50 amino acids, at least 60 amino acids, at least 70 amino acids, at least 80 amino acids, or at least 100 amino acids.

The AAHC protein (including a variant of a naturally-occurring AAHC protein) provided herein can be bound to a lipid or can be a lipid free apolipoprotein. For example, an AAHC protein can be isolated from an organism, from microorganism culture or tissue culture cells or media, or from bacterial cells engineered to express a recombinant AAHC protein. An AAHC protein can also by synthesized, for example, using chemical synthesis of peptides, optionally with peptide ligation to form larger peptides or proteins. The isolated apolipoprotein can be bound to lipid using methods known in the art (see, for example, Pownall et al. (1978) Biochemistry 17: 1183-1188; Pownall et al. (1981) Biochemistry 20: 6630-6635; Jonas et al. (1984) J. Biol. Chem. 259: 6369-6375; Jonas et al. (1989) J. Biol. Chem. 264: 4818-4824; Jonas et al. (1993) J. Biol. Chem. 268: 1596-1602; Tricerri et al. (2000) Biochemistry 39: 14682-14691; Segall et al. (2002) J. Lipid Res. 43: 1688-1700; Pearson et al. (2005) J. Biol. Chem. 280: 38576-38582, all incorporated by reference herein in their entireties).

### Phospholipid-Protein Particles (PPPs)

In some embodiments, scaffold proteins may be provided in IVPS systems that also include one or more lipids, such as but not limited to one or more phospholipids. The scaffold proteins in illustrative embodiments are recombinant scaffold proteins. Cholesterol, a cholesterol ester, or one or more other neutral lipids, such as, but not limited to, a sterol ester, a mono-, di-, or triacylglyceride, or an acylglycerol, can optionally also be included. Lipids can be present at a concentration of from about 1 microgram per milliliter to about 20 milligrams per milliliter, or from about 5 micrograms per milliliter to about 10 milligrams per milliliter, or from about 10 micrograms per milliliter to about 5 milligrams per milliliter. One or more phospholipids can be bound to a scaffold protein in the IVPS system. In some embodiments of the invention, apolipoproteins are translated using in vitro protein systems that include one or more lipids, such as but not limited to one or more phospholipids. The scaffold proteins synthesized in the cell-free system can bind one or more lipids during or following translation.

In some embodiments of the invention, scaffold proteins can be present in an IVPS system as phospholipid-protein particles (PPPs) in which the particles comprise phospholipids organized into a bilayer disc bound by the apolipoprotein or AAHC protein. Some examples of phospholipid-protein particles and methods of making phospholipid-protein discs (including phospholipid apolipoprotein disc that comprise apolipoprotein variants) are known in the art and described, for example, in Jonas et al. (1984) J. Biol. Chem. 259: 6369-6375; Jonas et al. (1989) J. Biol. Chem. 264: 4818-4824; Jonas et al. (1993) J. Biol. Chem. 268: 1596-1602; U.S. Pat. No. 7,048,949; U.S. Patent Application Publication No. 2005/0182243 A1, 2005/0152984 A1, 2004/0053384 A1, and 2006/0088524 A1, all incorporated by reference herein in their entireties.

Nanoscopic bilayer discs, herein disclosed as phospholipid-protein particles, or “PPPs”, are described, for example, in Jonas et al. (1982) Biochemistry 21: 6867-6872; Jonas et al (1986) Methods in Enzymology 128: 553-582; Zorich et al. (1987) Biochemica Biophysica Acta 919: 781-789; McGuire et al. (1996) J. Lipid Res 37: 1519-28; Bayburt et al. (1998); J. Structural Biology 123: 37-44; Rogers et al (1998) Biochemistry 37: 11714-25; Garda et al. (2002) J. Biological Chemistry 277: 19773-82; and in U.S. Pat. No. 7,048,949, U.S. Pat. No. 7,083,958; U.S. Patent Application Publication Nos. 2005/0182243, 2005/0152984, 2004/0053384, and WO 02/040501, all of which are incorporated by reference in their entireties, and in particular for disclosure of nanoscopic phospholipids bilayer discs, their components, their manufacture, methods of isolation of nanoscale phospholipid bilayer discs; methods of measuring the dimensions and analyzing the structure of nanoscale phospholipid bilayer discs; and methods of use. The methods of the invention produce membrane proteins that are inserted into phospholipid-protein particles, or nanoscopic phospholipid bilayer discs. A nucleic acid template is added to an IVPS system that comprises a cell extract and a preparation of PPPs; and the IVPS system is incubated to synthesize a membrane protein in soluble form, in which the membrane protein in soluble form is inserted into PPPs.

The present invention includes translation systems and methods comprising phospholipid bilayer particles or discs that include a scaffold protein such as a scaffold protein. Preferably the scaffold protein provided as a phospholipid-protein has at least one amphipathic helical domain. Illustrative examples include apolipoproteins, pepbiols, apomyoblobin, and synucleins (e.g., synuclein alpha (SEQ ID NO:84), synuclein alpha (SEQ ID NO:85), synuclein beta (SEQ ID NO:86), synuclein beta (SEQ ID NO:87), synuclein gamma (SEQ ID NO:88)).

The apolipoprotein can be, for example, Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, Lipoprotein (a), Apolipophorin I, Apolipophorin II, or Apolipophorin III or derivatives or variants thereof (for example, chimeric apolipoproteins, C-terminal or N-terminal truncated apolipoproteins, internally deleted apolipoproteins, apolipoproteins comprising additional amino acid sequences or altered amino acid sequences). In preferred embodiments, a phospholipid-apolipoprotein particle in an IVPS is Apo A-I, Apo A-IV, Apo A-V, Apo C-I, Apo C-II, Apo C-III, Apo-E, or Apolipophorin III, or a variant of any of these. In some embodiments, the length of an amphipathic helical domain of any apolipoprotein or AAHC protein can be altered to promote the formation phospholipid-protein particles of different desired diameters. This can be advantageous for accommodating multiple proteins within a phospholipid-protein particle.

Phospholipids used to form phospholipid-protein particles or discs in translation systems can be glycerol or sphingolipid based, and can contain, for example, two saturated fatty acids of from 6 to 20 carbon atoms and a commonly used head group such as, but not limited to, phosphatidyl choline, phosphatidyl ethanolamine and phosphatidyl serine. The head group can be uncharged, positively charged, negatively charged or zwitterionic. The phospholipids can be natural (those which occur in nature) or synthetic (those which do not occur in nature), or mixtures of natural and synthetic. Nonlimiting examples of phospholipids include, without limitation, PC, phosphatidyl choline; PE, phosphatidyl ethanolamine, PI, phosphatidyl inositol; DPPC, dipalmitoyl-phosphatidylcholine; DMPC, dimyristoyl phosphatidyl choline; POPC, 1-palmitoyl-2-oleoyl-phosphatidyl choline; DHPC, dihexanoyl phosphatidyl choline, dipalmitoyl phosphatidyl ethanolamine, dipalmitoyl phosphatidyl inositol; dimyristoyl phosphatidyl ethanolamine; dimyristoyl phosphatidyl inositol; dihexanoyl phosphatidyl ethanolamine; dihexanoyl phosphatidyl inositol; 1-palmitoyl-2-oleoyl-phosphatidyl ethanolamine; or 1-palmitoyl-2-oleoyl-phosphatidyl inositol; among others.

In addition to phospholipids, any of cholesterol, sphingolipids, glycolipids, lipopolysaccharides, ceramides, steroids, fatty acids, including derivatized versions or synthetic versions of these molecules, including but not limited to labeled analogs, can be incorporated into PPPs. Various hydrophobic or lipophilic molecules, or molecules with hydrophobic or lipophilic domains that can embed in a membrane bilayer, can be incorporated into the PPPs used in the methods and compositions of the invention.

The isolated apolipoprotein or AAHC protein and phospholipids can be mixed to assemble into phospholipid-protein particle, for example, as described in the art, including Jonas et al. (1984) J. Biol. Chem. 259: 6369-6375; Jonas et al. (1989) J. Biol. Chem. 264: 4818-4824; Jonas et al. (1993) J. Biol. Chem. 268: 1596-1602; U.S. Pat. No. 7,048,949; U.S. Pat. No. 7,083,958; U.S. Patent Application Publication No. 2005/0182243 A1, 2005/0152984 A1, 2004/0053384 A1, and 2006/0088524 A1, all incorporated by reference herein in their entireties, and in particular for methods of making and analyzing phospholipid-protein particles. The phospholipid-protein particles are then added to a cell extract or IVPS system.

In some other aspects of the invention, a nucleic acid construct encoding an scaffold protein is provided in an IVPS system that includes one or more phospholipids, and the scaffold protein translated in vitro associates with phospholipid to form a phosphophospholipid-protein particle in the IVPS system.

### Proteins of Interest (POI)

Proteins of interest (POI) that can be synthesized in vitro using the compositions and methods of the invention can be any proteins, and can be naturally-occurring proteins, sequence variants of naturally-occurring proteins, or engineered proteins, including fusion proteins, chimeric proteins, or proteins with sequences based on theoretical models. The protein synthesized using the methods and compositions of the invention can be any type of protein, for example, an enzyme, structural protein, carrier protein, transporter, receptor (e.g., a G protein-coupled receptor, a tyrosine kinase receptor, a cytokine receptor, etc.), ion channel protein, G protein, pore-forming protein, adhesion protein (e.g., a cell adhesion molecule (CAM) or substrate adhesion molecule (SAM)) hormone, growth factor, inhibitor, or activator.

Of particular interest are hydrophobic proteins and membrane proteins that are difficult to solubilized and isolate in the absence of denaturants, such as denaturing detergents. A membrane protein can be a transmembrane protein, an embedded membrane protein, or a peripheral membrane protein. Membrane proteins can be proteins with one or more membrane spanning domains, such as membrane spanning alpha helical domains. A membrane protein can also be a protein that associates with membranes.

A membrane protein can be a receptor protein. A receptor protein synthesized using the compositions and methods of the invention can be, for example, a receptor protein-tyrosine kinase (e.g., an insulin receptor, an EGF receptor, an NGF receptor, a PDGF receptor), a cytokine receptor (e.g., an interleukin-2 receptor, an erythropoietin receptor), or a G protein coupled receptor. G protein-coupled receptors can be of any class or family of GPCR, for example, a G protein-coupled receptor can be a Class A “rhodopsin-like” GPCR, a Class B “Secretin-like” GPCR, a Class C “Metabotropic glutamate/pheromone” GPCR, a Class D “Fungal pheromone” GPCR, a Class E “cAMP receptor” GPCR, a member of the “Frizzled/Smoothened family of GPCRs, or a taste receptor GPCR. A receptor can be, as illustrative and nonlimiting examples, a muscarinic acetylcholine receptor, an alpha adrenoceptor, a dopamine receptor, a histamine receptor, a serotonin receptor an octopamine receptor, a trace amine receptor, an angiotensin receptor, a bombesin receptor, a bradykinin receptor a C5a anaphylatoxin receptor, and Fmet-leu-phe receptor, an APJ like receptor, an interleukin receptor, a C—C chemokine receptor, a C—X—C chemokine receptor, a C—X3-C chemokine receptor, a C—C chemokine receptor, an opioid receptor, a somatostatin receptor, a tachykinin receptor, a vasopressin receptor, a urotensin receptor, and adrenomedullin receptor, an FSH receptor, a gonadotropin receptor, rhodopsin, an olfactory receptor, a prostaglandin receptor, and adenosine receptor, a cannaboid receptor, a purinoceptor, a platelet activating factor receptor, a gonadotropin-releasing hormone receptor, and the like.

A suitable POI is a hydrophobic protein that is not typically expressible at high levels in a soluble form. For example, membrane proteins are often difficult to isolate using bacterial (e.g., E. coli) expression systems. Many such proteins are known in the art. Exemplary proteins include but are not limited to enzymes, structural proteins, carrier proteins, transporters, receptors (e.g., a G protein-coupled receptor, a tyrosine kinase receptor, a cytokine receptor, etc.), ion channel proteins, G proteins, pore-forming proteins, adhesion proteins (e.g., a cell adhesion molecule (CAM) or substrate adhesion molecule (SAM)), hormones, growth factors, inhibitors, or activators. Additional non-limiting examples include, for example, EmrE (SEQ ID NO: 43), bacteriorhodopsin (SEQ ID NO: 44), a polypeptide expressible from the Invitrogen Ultimate™ ORF clone collection (www.invitrogen.com), a G protein-coupled receptor (GPCR), G protein-coupled receptor family C group 5 member C (IOH5520; NM_{—}022036; SEQ ID NO: 45), G protein-coupled receptor 157 (BC018691.1; SEQ ID NO: 46), serotonin receptor HTR1 (IOH46452; SEQ ID NO: 47), endothelin receptor type B (IOH14234; NM_{—}000115.1; SEQ ID NO: 48), opiate receptor-like 1 (IOH 27433; NM_{—}000913.3; SEQ ID NO: 49), cholinergic receptor muscarinic 2 (IOH28351; NM_{—}000739.2; SEQ ID NO: 50), histamine receptor H2 (IOH28904; BC054510.2; SEQ ID NO: 51), dopamine receptor D1 (IOH29556; NM_{—}000794.3; SEQ ID NO: 52), melanocortin 5 receptor (IOH29738; NM_{—}005913.1; SEQ ID NO: 53), corticotropin releasing hormone receptor 1 (IOH39398; NM_{—}004382.2; SEQ ID NO: 54), 5-hydroxytryptamine (serotonin) receptor 1A (IOH46452; NM_{—}000524.2; SEQ ID NO: 55), cholinergic receptor muscarinic 1 (IOH56940; NM_{—}000738.2; SEQ ID NO: 56), CD24 (IOH5911; NM_{—}013230.2; SEQ ID NO: 57), glycophorin E (IOH12322; BC017864.1; SEQ ID NO: 58), glycophorin B (NM_{—}002100.3; SEQ ID NO: 59; IOH58935), chemokine-like factor (IOH58583; NM_{—}181640.1; SEQ ID NO: 60), glycophorin A (IOH7353; BC005319.1; SEQ ID NO: 61), murine microsomal glutathione S-transferase 1 (IOM19680; BC009155.1; SEQ ID NO: 62), phosphatidylinositol glycan anchor biosynthesis class P (IOH44755; NM 153681.2; SEQ ID NO: 63), epiregulin (IOM14930; NM_{—}007950.1; SEQ ID NO: 64), epiregulin (IOH42289, IOH58999; NM_{—}001432.2; SEQ ID NO: 65), CD99 (IOH5089; NM_{—}002414.3; SEQ ID NO: 66), murine Mpv17 transgene (IOM15042; NM_{—}008622.2; SEQ ID NO: 67), MpV17 mitochondrial inner membrane protein (IOH3860; NM_{—}002437.4; SEQ ID NO: 68), translocase of inner mitochondrial membrane 22 homolog (IOH3712; NM_{—}013337.2; SEQ ID NO: 69), ninjurin 2 (IOH43470; NM_{—}016533.4; SEQ ID NO: 70), signal peptide peptidase-like 2B (IOH4396; BC001788.1; SEQ ID NO: 71), CKLF-like MARVEL transmembrane domain containing 1 (IOH58697; NM_{—}181268.2; SEQ ID NO: 72), golgi transport 1 homolog B (IOH10546; NM_{—}016072.3; SEQ ID NO: 73), leukotriene C4 synthase (IOH54642; NM_{—}145867.1; SEQ ID NO: 74), angiotensin II receptor-associated protein (IOH 14721; NM_{—}001040194.1; SEQ ID NO: 75), arachidonate 5-lipoxygenase-activating protein (IOH11710; NM_{—}001629.2; SEQ ID NO: 76), signal peptide peptidase 3 (IOH11788; NM_{—}025781.1; SEQ ID NO: 77), leptin receptor (IOH13675; NM_{—}017526.2; SEQ ID NO: 78), microsomal glutathione S-transferase 3 (IOH7518; NM_{—}004528.2; SEQ ID NO: 79), dystrobrevin binding protein 1 (IOH26587; NM_{—}033542.2; SEQ ID NO: 80), PRA1 domain family member 2 (IOH57177; NM_{—}007213.1; SEQ ID NO: 81), phosphatidic acid phosphatase type 2 domain containing 1B (IOH54702; NM_{—}032483.3; SEQ ID NO: 82), and human adrenomedullin receptor protein (SEQ ID NO: 83). Fragments, variants, and derivatives of POIs are also contemplated herein.

As described herein, POIs may also be co-expressed or complexed with other proteins such as chaperonins or subunits normally expressed with the POI in a cell. Suitable chaperonins include, for example, general chaperones such as BiP (e.g, NP_{—}005338, NP_{—}071705), GRP94 (e.g., NP_{—}003290, NP_{—}035761), and/or GRP170; lectin chaperones such as calnexin (e.g., NP001019820, NP_{—}031623) and calreticulin (e.g., NP_{—}004334, NP_{—}031617); non-classical chaperones such as HSP47 (e.g., NP_{—}001226, XP_{—}994015) and ERp29 (e.g., NP_{—}001029197, NP_{—}080405); heat shock proteins such as Hsp10 (e.g., NP 002148, NP_{—}032329), Hsp27 (e.g., NP_{—}001531, NP_{—}038588), Hsp47 (e.g., NP_{—}001226, XP_{—}994015), Hsp60 (e.g., NP_{—}002147, NP_{—}034607), Hsp70 (NM_{—}005345), Hsp90 (HUGO Code HSP90AA1), or Hsp100; folding chaperones such as protein disulfide isomerase (PDI) (e.g., NM_{—}006849, NM_{—}005313, NM_{—}004911, NM_{—}006810, NM_{—}005742), peptidyl prolyl cis-trans-isomerase (PPI), or ERp57 (NM_{—}005313); and/or bacterial chaperonins such as GroEL or GroES or their mammalian homologs (e.g., NP_{—}002147, NP_{—}034607, NP_{—}002148, NP_{—}032329). Other suitable accessory proteins may also be utilized.

Functional domains of POIs may also be utilized, either alone or as fusion proteins with other proteins that may serve to anchor the domain within the PPP. POIs may also be expressed as fusion proteins with other proteins such as those tagged with, for example, a fluorescent tag (e.g., Green Fluorescent Protein (GFP)) for utilization in detection assays (e.g., FRET assays). POIs may also be expressed along with some or all of the subunit proteins the POI with which the POI is normally expressed in cells.

### Recombinational Cloning

Cloning systems that utilize recombination at defined recombination sites, including the GATEWAY® recombination cloning system, vectors, enzymes, and kits available from Invitrogen (Carlsbad, Calif.) have been previously described in U.S. application Ser. No. 09/177,387, filed Oct. 23, 1998; U.S. application Ser. No. 09/517,466, filed Mar. 2, 2000; and U.S. Pat. Nos. 5,888,732 and 6,277,608, all of which are specifically incorporated herein by reference. These systems can be used for cloning MPOI coding sequences and/or apolipoprotein coding sequences into expression vectors for in vitro translation, and multisite GATEWAY® vectors can be used to accommodate multiple open reading frames for simultaneous translation of two or more proteins in a single reaction.

In brief, the GATEWAY® Cloning System utilizes vectors that contain at least one recombination site to clone desired nucleic acid molecules in vivo or in vitro. More specifically, the system utilizes vectors that contain at least two different site-specific recombination sites based on the bacteriophage lambda system (e.g., att1 and att2) that are mutated from the wild-type (att0) sites. Each mutated site has a unique specificity for its cognate partner att site (i.e., its binding partner recombination site) of the same type (for example, attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the GATEWAY® cloning system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

### Methods and Systems for Synthesizing Proteins in Vitro Using Scaffold Proteins

The present invention provides efficient systems and methods for synthesizing membrane proteins in a cell-free system in soluble form. The methods include translating membrane proteins in a cell free system that includes phospholipid-protein particles.

The present invention is based on the finding that membrane proteins can insert into phospholipid-protein particles (phospholipids bilayer discs) when the membrane proteins are translated in the presence of phospholipid-protein particles (PPPs). As illustrated in the Examples provided herein, synthesis of a membrane POI (MPOI) in an IVPS (IVPS) system that contains PPPs results in production an MPOI with enhanced solubility, in which the MPOI is incorporated into PPPs.

It has also been determined that membrane proteins may be translated in the presence of a scaffold protein such as an apolipoprotein or AAHC that is not part of a PPP, in which the MPOI translated in the presence of an apolipoprotein has enhanced solubility with respect to the same MPOI translated in vitro in the absence of the scaffold protein. The invention thus includes in vitro synthesis methods and systems for translating proteins in the presence of the scaffold protein. The invention includes in vitro synthesis methods and systems for translating proteins in the presence of a scaffold protein in which the scaffold protein in the IVPS system is not provided in a PPP. The invention also includes in vitro synthesis methods and systems for translating proteins in the presence of a scaffold protein in which exogenous phospholipids are not present in the IVPS system.

It has also been determined that the scaffold protein may be translated in the same IVPS system in which an MPOI is translated, and when both the MPOI and the scaffold protein are synthesized in the same IVPS reaction, the MPOI has enhanced solubility with respect to its solubility when synthesized in an IVPS reaction that does not contain the scaffold protein or does not include a nucleic acid template encoding the scaffold protein.

In one aspect, then, the invention provides a method of synthesizing a POI in vitro, comprising: adding a nucleic acid template that encodes a POI to an IVPS system that includes a scaffold protein such as a scaffold protein, or a nucleic acid template encoding the scaffold protein, and incubating the IVPS system to synthesize the POI. In some preferred embodiments, the POI is synthesized in soluble form. In some preferred embodiments, the POI is a membrane protein or hydrophobic protein. In preferred embodiments, the POI is a hydrophobic protein such as a membrane protein, and a majority (51% or greater) of the protein synthesized in the IVPS system that includes a scaffold protein such as a scaffold protein is synthesized in soluble form. In preferred embodiments, the percentage of soluble protein synthesized (with respect to total protein synthesized) in the IVPS system that includes the scaffold protein is higher than the percentage of soluble protein synthesized in an IVPS system that does not include the scaffold protein.

As described above, a POI translated in the IVPS system can be any POI, such as an enzyme, G protein, ion channel protein, pore-forming protein, cell adhesion protein, substrate adhesion protein, receptor, G protein-coupled receptor, structural protein, carrier protein, binding protein, antibody, hormone, growth factor, inhibitor, or activator. In some embodiments, the protein synthesized in the in vitro system is not a membrane protein. The Examples provided herein demonstrate the presence of apolipoprotein in an IVPS reaction does not deleteriously affect translation of non-membrane proteins. In some preferred embodiments, a POI translated using the methods of the invention is a membrane protein (“MPOI”), or a protein that in its native state associates with biological membranes, such as, for example, a transmembrane protein, an embedded membrane protein, or a peripheral membrane protein. Nonlimiting examples of membrane proteins are provided herein.

In some preferred embodiments, a POI translated using the methods of the invention is a membrane protein, and after incubating the IVPS system a majority (51% or greater) of the synthesized protein is in soluble form. In some preferred embodiments, a POI translated using the methods described herein is a membrane protein, and after incubating the IVPS system a larger amount of the membrane POI (MPOI) is synthesized in soluble form than when the protein is translated in the absence of the scaffold protein. For example, in preferred embodiments at least 10%, at least 20%, at least 30%, at least 40%, at least 50%, at least 60%, at least 70%, at least 80%, at least 90% or at least 100% more of the MPOI is synthesized in soluble form in the presence of a scaffold protein such as a scaffold protein (or when the scaffold protein is being translated in the same in vitro synthesis system) than when there is no scaffold protein present (i.e., as pre-formed protein or as a co-translated expression product) in the IVPS reaction. In some preferred embodiments, after incubating the IVPS system that includes a scaffold protein or a nucleic acid template encoding a scaffold protein with a nucleic acid template encoding a MPOI under conditions that promote protein synthesis, there is a higher percentage of soluble MPOI to total POI synthesized than when the MPOI is translated in the absence of the scaffold protein, or a nucleic acid template encoding the scaffold protein. For example, in preferred embodiments the percentage of soluble MPOI to total MPOI synthesized in an IVPS reaction increases by at least 10%, at least 20%, at least 30%, at least 40%, at least 50%, at least 60%, at least 70%, at least 80%, at least 90% or at least 100% when the MPOI is synthesized in the presence of the scaffold protein with respect to the percentage of soluble MPOI to total MPOI synthesized when the MPOI is synthesized without scaffold protein being present in the IVPS reaction.

As described herein, a scaffold protein such as a scaffold protein provided in an IVPS system is a protein that is either a naturally-occurring apolipoprotein or other AAHC protein such as MSP1 (SEQ ID NO: 20), synuclein alpha (SEQ ID NO:83), synuclein alpha (SEQ ID NO:84), synuclein beta (SEQ ID NO:85), synuclein beta (SEQ ID NO:86), synuclein gamma (SEQ ID NO:87), apomyoglobin, a peptabiol, melitin, almethicin, and gramicidin, of any species origin; a sequence variant thereof, or, an engineered protein having at least one alpha helical domain that has at least 90% homology to an alpha helical domain of a naturally-occurring apolipoprotein or AAHC protein. Scaffold proteins such as apolipoproteins and AAHC proteins used in the methods and compositions of the present invention have the property of increasing the soluble yield of a membrane protein by at least 10%, where the soluble yield is calculated as either the amount of soluble protein synthesized, or the percentage of soluble protein to total protein synthesized, when the scaffold proteins are provided in an IVPS system or translated in an IVPS that is also translating the membrane protein.

A scaffold protein such as a scaffold protein that is present in an IVPS system can be present at any concentration that permits translation of a MPOI. As general guidelines only, the scaffold protein may be provided in an IVPS system at concentration of from about 0.5 micrograms per mL to about 2 milligrams per mL, or from about 1 microgram per mL to about 1 mg per mL, or from about 5 micrograms per mL to about 500 micrograms per mL, or from about 10 micrograms per mL to about 250 micrograms per mL. More than one scaffold protein may be present in a single IVPS reaction.

The one or more scaffold proteins can be added to an IVPS reaction after a nucleic acid template is added to the reaction, but preferably a scaffold protein such as a scaffold protein is present in an IVPS reaction when a nucleic acid template encoding a POI is added. As used herein, “adding to an IVPS system” means adding to a cell extract prepared for IVPS, to which other components for in vitro synthesis (for example, amino acids, buffers, enzymes, cofactors, energy sources, tRNAs, labels, etc.) may have already been added, or are yet to be added.

In some embodiments, the methods further include isolating the POI from the IVPS mixture. Isolation procedures can be, for example, by means of a peptide tag that is part of the scaffold protein or by a peptide tag that is incorporated into the sequence of the POI, or by using a specific binding member, such as but not limited to an antibody, that binds a domain of the POI or scaffold protein.

The invention thus includes, in another aspect, a cell extract for in vitro translation that includes at least one scaffold protein as described herein. Cell extracts for in vitro translation include all those disclosed herein, and can be prokaryotic or eukaryotic. In some embodiments, the invention includes an IVPS system that includes a scaffold protein, a cell extract, and a chemical energy source. In some embodiments, the invention includes an IVPS system that includes a scaffold protein, a cell extract, a chemical energy source added to the extract, and one or more added amino acids. In some embodiments, the invention includes an IVPS system that includes a scaffold protein, a cell extract, a chemical energy source that has been added to the extract, one or more amino acids that have been added to the extract, and a nucleic acid template. The nucleic acid template can be a DNA or RNA template, and in some embodiments encodes a membrane protein. The IVPS system can optionally include one or more lipids, detergents, surfactants, salts, buffering compounds, enzymes, inhibitors, reducing agents, or cofactors.

In some embodiments of the methods of the invention, a scaffold protein is added to or present in an IVPS system that includes one or more lipids, such as but not limited to one or more phospholipids. In some embodiments of the methods of the invention, a scaffold protein is added to an IVPS system that includes one or more lipids and the scaffold protein becomes associated with one or more lipids in the IVPS system. In some embodiments, the scaffold protein is associated with one or more lipids when it is added to an IVPS system. In some embodiments, the scaffold protein is added to an IVPS system that includes one or more lipids, or the scaffold protein is associated with one or more lipids when it is added to an IVPS system, and during incubation of the IVPS system, a synthesized POI become associated with the scaffold and its associated lipid(s) in the IVPS system.

In some embodiments of the methods of the invention, a scaffold protein added to an IVPS system is added as a phospholipid-protein particle (PPP). In certain embodiments, a PPP includes one or more scaffold proteins and one or more phospholipids. In some embodiments of the methods of the invention, a scaffold protein added to an IVPS system is added as a PPP and a MPOI synthesized in the system becomes associated with a PPP, such that the MPOI synthesized in the system can be isolated with the PPP.

In a further aspect, therefore, the invention includes a cell extract for translation that includes phospholipid-protein particles (PPPs) as described herein. Cell extracts for in vitro translation include all those disclosed herein, and can be prokaryotic or eukaryotic. In some embodiments, the invention includes an IVPS system that includes PPPs, a cell extract, and a chemical energy source. In some embodiments, the invention includes an IVPS system that includes PPPs, a cell extract, a chemical energy source that has been added to the cell extract, and one or more added amino acids. In some embodiments, the invention includes an IVPS system that includes PPPs, a cell extract, an added chemical energy source, one or more added amino acids, and a nucleic acid template. The IVPS system can optionally include one or more lipids, detergents, salts, buffering compounds, enzymes, inhibitors, or cofactors.

Phospholipid-protein particles (PPPs) as described in detail above, can be added to or provided in an IVPS system in any concentration that permits in vitro translation, but is preferably added at a concentration that enhances the solubility of a MPOI translated in the IVPS. As general guidelines only, PPPs can be added at concentrations ranging from about 0.5 micrograms per mL to about 2 milligrams per mL, or from about 1 microgram per mL to about 1 mg per mL, or from about 5 micrograms per mL to about 500 micrograms per mL, or from about 10 micrograms per mL to about 250 micrograms per mL, where the concentration given is based on the protein content of the PPPs. More than one type of PPP can be present in a single IVPS reaction, where different PPPs have different scaffold proteins and/or different phospholipid compositions.

In yet another aspect of the invention, a scaffold protein can be provided in an IVPS system by translating the scaffold protein in the IVPS system that translates the POI. The invention provides a method of synthesizing a protein in vitro, in which the method includes: adding to an in vitro synthesis system a nucleic acid construct that encodes a scaffold protein and a nucleic acid construct that encodes a POI, and incubating the IVPS system to synthesize a scaffold protein and a POI. In some preferred embodiments, the POI is synthesized in soluble form. In some preferred embodiments, the POI is a membrane protein, as described hereinabove.

In some embodiments, a scaffold protein is provided on a first nucleic acid construct, and a POI is provided on a second nucleic acid construct. In other embodiments of this aspect of the invention, sequences encoding a scaffold protein and sequences encoding a POI are provided on the same nucleic acid construct. GATEWAY® vectors and cloning systems (Invitrogen, Carlsbad, Calif.) can optionally be used in making nucleic acid constructs that encode one or both of a scaffold protein and a POI. In some embodiments, a DNA construct that includes sequences encoding a scaffold protein and sequences encoding a POI has a first promoter for the apolipoprotein or AAHC protein coding sequences a second promoter for the POI coding sequences. In one alternative, a nucleic acid construct that includes sequences encoding a scaffold protein and sequences encoding a POI include an IRES sequence between the two coding sequences.

A nucleic acid construct encoding a scaffold protein can encode any apolipoprotein or AAHC as disclosed herein, including a naturally-occurring apolipoprotein or AAHC protein, a sequence variant of a naturally-occurring apolipoprotein or AAHC protein, or an engineered apolipoprotein or AAHC protein having at least one helical domain that has at least 70%, 80%, or 90% homology to a helical domain of a naturally-occurring apolipoprotein or AAHC protein. A nucleic acid construct encoding a scaffold protein may have an amino acid sequence that is modified with respect to the amino acid sequence of a wild-type scaffold protein. In some embodiments, a nucleic acid construct encoding a scaffold protein variant encodes a tag sequence fused to the scaffold sequence.

In some preferred embodiments, a POI translated in an IVPS that includes a template encoding a scaffold protein and a template encoding a membrane protein, and after incubating the IVPS system, a larger amount of the membrane POI (MPOI) is synthesized in soluble form than when the MPOI is translated in the absence of scaffold protein being present or produced under in vitro synthesis conditions that are otherwise the same. In preferred embodiments, the percentage of soluble protein synthesized (with respect to total protein synthesized) in the IVPS system that includes a scaffold protein is higher than the percentage of soluble protein synthesized in an IVPS system that does not include a scaffold protein. In preferred embodiments, a majority (51% or greater) of a membrane protein or hydrophobic protein is synthesized in the IVPS system that includes a scaffold protein is synthesized in soluble form.

In some embodiments, an IVPS system of the invention that comprises nucleic acid construct(s) encoding a POI and a scaffold protein comprises one or more lipids, such as but not limited to one or more phospholipids. For example, one or more phospholipids, such as, for example, DPPC, DOPC, POPC, or any others disclosed herein, can be present at a concentration of from about 1 microgram to 1 mg per mL, or from about 5 micrograms to about 800 micrograms per mL, or from about 10 to about 600 micrograms per mL, or from about 25 to about 500 micrograms per mL. For example, one or more phospholipids can be present at a concentration of from about 10 to about 50 micrograms per mL, from about 50 to about 100 micrograms per mL, from about 100 to about 200 micrograms per mL, from about 200 to about 300 micrograms per mL, from about 300 to about 400 micrograms per mL, from about 400 to about 500 micrograms per mL from about 500 to about 700 micrograms per mL, or from about 700 micrograms to about 1 mg per mL. In some embodiments, methods of the invention that comprise synthesizing a POI in soluble form comprise adding to an in vitro synthesis system that comprises at least one lipid a nucleic acid construct that encodes a scaffold protein and a nucleic acid construct that encodes a POI and incubating the IVPS system to synthesize a scaffold protein particle and a POI associated with the phospholipid-protein particle.

The invention thus also includes methods of making a protein-phospholipid particle, in which the method includes: synthesizing a protein that includes at least one amphipathic helix in vitro in the presence of phospholipid to make a protein phospholipid particle. The method includes adding a nucleic acid template to an in vitro protein synthesis system, in which the in vitro protein synthesis includes a cell extract, at least one exogenously added energy source, and phospholipid, and incubating the in vitro synthesis system to synthesize a protein-phospholipid particle.

The methods of making PPPs by providing components in an IVPS system can be combined with other embodiments described herein, including, use of a tagged apolipoprotein, translation of MPOIs with PPP components on arrays or multiwell plates, translation of two or more MPOIs with PPP components, inclusion of components of the protein translocation machinery in the IVPS reaction mix that includes PPPs or PPP components, and translation of one or more components of the protein translocation machinery in the IVPS reaction mix that also includes PPPs or PPP components.

The invention therefore provides, in a further aspect, an IVPS system that includes a cell extract, a nucleic acid template that encodes a scaffold protein, and a nucleic acid template that encodes a POI. In certain embodiments, the invention includes an IVPS system that includes a cell extract, a first nucleic acid molecule that encodes a scaffold protein, and a second nucleic acid molecule that encodes a POI. In other embodiments, an IVPS system that includes a cell extract and a nucleic acid template that encodes a scaffold protein and a POI. Either or both of the nucleic acid templates can be DNA or RNA.

A construct that encodes a scaffold protein to be translated in an IVPS system can also encode an amino acid tag fused in frame with the scaffold protein sequence. A nucleic acid template that encodes a scaffold protein can be a DNA template or an RNA template. A nucleic acid template that encodes an apolipoprotein can be bound to a solid support, such as, for example, a bead, matrix, chip, array, membrane, sheet, dish, or plate.

A nucleic acid template that encodes a POI can be a DNA template or an RNA template, and can encode any POI of any species, such as but not limited to an enzyme, structural protein, carrier protein, hormone, growth factor, receptor (e.g., a GPCR, tyrosine kinase receptor, cytokine receptor, etc.), adhesion molecule, channel protein, pore-forming protein, transporter, inhibitor, or activator. In some preferred embodiments, a POI translated using the methods of the invention is a membrane protein. A construct that encodes a POI can also encode an amino acid tag fused in frame with the POI sequence. An amino acid tag can be an affinity tag, as disclosed herein, or can be a “self-labeling tag”, such as, for example, a LUMIO® tag (FlAsH or ReAsH tag), a Halotag, or a SNAP-tag.

A nucleic acid construct present in an IVPS system of the invention can encode more than one POI. A nucleic acid template that encodes a POI can be bound to a solid support, such as, for example, a bead, matrix, chip, array, membrane, sheet, dish, or plate.

### Use of Affinity Tags

The invention also provides methods for efficient systems and methods for in vitro synthesis of membrane proteins in soluble and readily purifiable form. In these methods, an MPOI is synthesized in an in vitro translation reaction that includes a scaffold protein, in which the scaffold protein has a purification tag. Capture of the scaffold protein using the purification tag leads to the co-isolation of membrane proteins synthesized in vitro in the presence of the apolipoprotein. In embodiments in which the scaffold protein is incorporated into a PPP, capture of the scaffold protein using the purification tag leads to isolation of PPPs that include the MPOI. The PPPs having incorporated MPOIs can be used for any of a number of assays, and also for structural studies, such as but not limited to NMR or X-ray crystallography.

In another embodiment, a membrane POI (MPOI) can optionally be translated in the presence of a scaffold protein, or can be co-translated with a scaffold protein, in which the MPOI has a protein tag attached for further identification, isolation, tethering, or purification or immobilization of the synthesized protein. In this case, the scaffold protein can optionally also have a tag.

The invention includes methods of synthesizing a membrane protein or hydrophobic protein in vitro, in which the membrane protein or hydrophobic protein is synthesized in an IVPS system that includes scaffold protein that includes an affinity tag. The scaffold protein can be present in a PPP. An affinity tag is, in preferred embodiments, a peptide sequence that can be used for labeling, immobilizing, separating, or purifying a protein by binding of a specific binding reagent to the affinity tag. Examples of tags that can be incorporated into proteins for capture or detection of the synthesized membrane or hydrophobic protein using an affinity reagent include, without limitation, his tags comprising multiple (four or more, typically six) histidines, FLAG tag, hemaglutinin tag, myc tag, glutathione-S-transferase, maltose binding protein, calmodulin, chitin binding protein, a HAT sequence, a T7 gene 10 sequence, etc. Another amino acid sequence tag is a tetracysteine-containing lumio tag that can be used for purification or detection of a protein using a tetraaresenical or biarsenical reagent (see, e.g., U.S. Pat. Nos. 6,054,271; 6,008,378; 5,932,474; 6,451,569; WO 99/21013, which are incorporated into the present disclosure by reference). A tag can also be a chemical moiety that can be bound by an affinity reagent, for example, biotin or nitroloacetic acid (NTA).

Capture of the AAHC protein using a reagent that specifically binds the affinity tag leads to isolation of PPPs that include the in vitro synthesized membrane protein or hydrophobic POI. The affinity reagent can be attached to any solid or semi-solid support, such as, for example, a column matrix, resin, gel, bead, membrane, filter, chip, slide, well, dish, chip, or array. The affinity reagent also can be a label, such as a fluorescent label, that is used to separate PPPs by detection of a labeled fraction in chromatographic separation or by flow cytometry. PPPs that are separated or purified using an affinity tag can be used for assays for binding or activity of the synthesized membrane protein or hydrophobic protein, or can be used for structural studies of the POI, such as, for example, NMR spectroscopy or X-ray crystallography.

The present invention further provides methods for in vitro synthesis of POIs, including MPOIs, where the identity of the proteins may be known or unknown, in IVPS reactions that include scaffold proteins (in the context of PPPs or not in PPPs), in which multiple reactions are performed in parallel, for example, in a multiwell plate to obtain multiple solubilized proteins for assays. The proteins can be expressed from vector-driven templates, where the vectors include transcriptional and translational expression sequences located near cloning sites. The vectors can be used to clone libraries of sequences, and can optionally include protein tag sequences that can be translated in frame with the POIs.

In one preferred embodiment, a scaffold protein of a PPP can include an affinity tag (such as a his tag, glutathione tag, streptavidin tag, etc.) used to tether the PPP containing a MPOI to a solid support, such as but not limited to a microwell surface, a chip surface, a sheet, a membrane, a matrix or bead. MPOIs translated with PPPs can be immobilized to a microwell, chip surface, sheet, membrane, matrix, or bead via their insertion into the tethered PPPs. The PPP can be tethered to the solid support before or after translation of the MPOI in the presence of the PPP.

Thus, the methods of the present invention can be used to make membrane protein arrays or multiwell assay plates, where localized in vitro translation reactions that include PPPs allow for tethering of PPPs having individual MPOIs inserted to specific locations on the array. Such arrays can be used for many types of screens and assays, including but not limited to enzymatic assays, ion channel assays, and drug binding assays. Labeling of MPOIs in the translation reaction, as described below, can be performed for facilitating array assays.

The arrays or multiwell assay plates can be made by in vitro translation reactions that are performed on the array or plate itself. For example, each location on an array, or well or a plate, can receive an IVPS reaction that includes a cell extract, PPPs, and a nucleic acid template that encodes an MPOI. The PPPs can become tethered to the array via a histidine tag, glutathione, streptavidin, or other tag engineered into the apolipoprotein. An MPOI can be a known or unknown protein.

In another embodiment the MPOI can be engineered to include a tag, for example, it can be cloned into a vector that provides a sequence that encodes a tag as an N-terminal or C-terminal amino acid sequence of the POI. The tag can be used for further isolation, tethering, or purification or immobilization of the proteins, which can be translated in the presence of a scaffold protein that can be provided without associated phospholipids, or in the context of PPPs. The synthesized protein can be captured, for example, to the bottom of a well, or an array locus or well, or to a filter, matrix, or bead, that has been treated or coated with an affinity capture reagent.

In yet other embodiments, the invention includes methods of making PPPs that include lipids that include affinity tags. For example, biotin can be conjugated to lipids, such as phospholipids and PPPs that contain the biotin-functionalized lipids can be isolated by their binding to avidin (see, for example, Peker et al. (2004) “Affinity Purification of Lipid Vesicles” Biotechnol. Prog. 20: 262-268). The invention includes methods of making PPPs that include combining a scaffold protein, a phospholipid, at least one lipid comprising an affinity tag, and detergent; incubating the mixture; and removing the detergent from the mixture to produce PPPs that include the scaffold protein, phospholipid, and at least one lipid comprising an affinity tag. The lipid that includes the affinity tag can be a phospholipid (e.g., DPPC, DOPC, POPC, etc.) or can be another type of lipid, such as, for example, a sphingolipid or a glycolipid that is incorporated into the PPPs. In some embodiments, the methods further include isolating the PPPs using an affinity reagent that binds the affinity tag. The affinity reagent can be bound to a solid or semi-solid support, for example, a column matrix, resin, gel, bead, plate, slide, well, chip, array, filter, or membrane.

In some embodiments, the methods include methods of making PPPs that include translating a scaffold protein in the presence of phospholipid and at least one lipid comprising an affinity tag to produce PPPs that include the scaffold protein, phospholipid, and at least one lipid comprising an affinity tag. The lipid that includes the affinity tag can be a phospholipid (e.g., DPPC, DOPC, etc) or can be another type of lipid, such as, for example, a sphingolipid or a glycolipid that is incorporated into the PPPs. In some embodiments, the methods further include isolating the PPPs using an affinity reagent that binds the affinity tag. The affinity reagent can be bound to a solid or semi-solid support, for example, a column matrix, resin, gel, bead, plate, slide, well, chip, array, filter, or membrane.

In other embodiments, the invention includes methods of making PPPs that include at least one membrane protein or at least one hydrophobic protein that include: combining a scaffold protein, at least one POI (e.g., membrane protein or hydrophobic protein), phospholipid, at least one lipid comprising an affinity tag, and detergent; incubating the mixture; and removing the detergent from the mixture to produce PPPs that include the scaffold protein, at least one membrane protein or protein, phospholipid, and at least one lipid comprising an affinity tag. In yet other embodiments, methods are provided for synthesizing a membrane protein or POI in soluble form, in which the methods include translating a membrane protein or POI in an in vitro protein synthesis system that includes PPPs having incorporated lipids that include an affinity tag. In yet other embodiments, methods are provided for synthesizing a membrane protein or hydrophobic protein in soluble form, in which the methods include translating a scaffold protein and a membrane protein or POI in an in vitro protein synthesis system that includes phosopholipid and at least one lipid comprising an affinity tag. In all of these methods, the lipid that includes the affinity tag can be a phospholipid (e.g., DPPC, DOPC, etc), which can be the same or different from the predominant phospholipid that constitutes the PPP, or can be another type of lipid, such as, for example, a sphingolipid or a glycolipid that is incorporated into the PPPs. In some embodiments, the methods further include isolating the PPPs using an affinity reagent that binds the affinity tag. The affinity reagent can be bound to a solid or semi-solid support, for example, a column matrix, resin, gel, bead, plate, slide, well, chip, array, filter, or membrane.

### Incorporation of Labels

The invention also includes methods of translating membrane proteins or hydrophobic proteins in an IVPS system that includes a scaffold protein (or an IVPS system that co-translates a scaffold protein) in which the MPOIs are labeled during translation, such as, for example, with a radiolabel, a heavy isotope label, or a fluorescent label (such as BODIPY® FL fluorophore incorporated at the N-terminus through inclusion of tRNA met (fmet) misaminoacylated with a methionine containing a BODIPY® FL fluorophore at its amino group). Alternatively, MPOIs can be engineered to contain a tag that can bind a label, such as, for example, a fluorescent label (as nonlimiting examples, LUMIO™ tetracysteine sequence motif detection technology can be used (Invitrogen, Carlsbad, Calif.; see for example US 2003/0083373, U.S. Pat. No. 5,932,474, U.S. Pat. No. 6,008,378, U.S. Pat. No. 6,054,271, WO 99/21013, all herein incorporated by reference in their entireties) or PRO-Q® Sapphire 532, 365, or 488 Oligohistidine stain for his-tagged proteins (Invitrogen, Carlsbad, Calif.). The method includes: translating a membrane protein in an in vitro synthesis reaction that includes a scaffold protein and at least one label that can be incorporated into the synthesized membrane protein. In an alternative embodiment, the method includes: translating a membrane protein in an in vitro synthesis reaction that includes at least one apolipoprotein or AAHC protein where the translated membrane protein includes at least one tag that can bind a label. The methods result in the production of labeled or tagged membrane proteins in soluble form. The method in preferred embodiments results in production of a tagged and/or labeled membrane protein membrane protein having enhanced solubility. In certain illustrative aspects of the invention, the labeled PPPs of the invention, such as PAPs of the invention, include a labeled phospholipid, such as a fluorescently labeled phospholipid. In order to form such labeled PPPs, a labeled phospholipid can be added, for example, into an in vitro translation reaction mixture.

Using the methods described herein, a POI may be synthesized in soluble form in an in vitro synthesis system that includes a scaffold protein so that the membrane protein or hydrophobic protein incorporates one or more labeled amino acids. The labeled amino acids can be labeled with one or more radioisotopes, heavy atoms, or heavy isotopes. The labeled amino acids can also be labeled with one or more fluorophores.

A POI translated using the methods described herein may be a fusion protein, in which the POI is linked to a fluorescent protein, such as green fluorescent protein or any of its derivatives or mutants, or any other fluorescent protein. For example, sequences encoding GFP, EGFP, BFP, CFP, RFP, or YFP or fluorescent variants thereof, can be fused to a sequence encoding a POI.

In some preferred embodiments of these methods, the scaffold proteins present in the IVPS system are in PPPs. The invention therefore includes translating a POI in an in vitro synthesis reaction that includes phospholipid-protein particles and at least one label that can be incorporated into the synthesized membrane protein to produce a labeled POI in soluble form. The method includes: translating a POI in an in vitro synthesis reaction that includes phospholipid-protein particles and at least one label that can be incorporated into the synthesized membrane protein or hydrophobic protein to produce a labeled membrane protein inserted into phospholipid-protein particles. In an alternative embodiment, the method includes: translating a POI in an in vitro synthesis reaction that includes at least one phospholipid-protein particle, in which the translated POI includes at least one tag that can bind a label. The method includes: translating a POI in an in vitro synthesis reaction that includes phospholipid-protein particles, in which the translated membrane protein or hydrophobic protein includes at least one tag that can bind a label to produce a tagged membrane protein or tagged hydrophobic protein inserted into phospholipid-protein particles.

A label can be, without limitation, a fluorescent label (e.g., fluoroscein, FITC, rhodamine, B-phycoerythrin, R-phycoerythrin, Texas Red, allophycocyanin, Cy3, Cy5, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and Alexa Fluor 750, DyLight 405, DyLight 488, DyLight 549, DyLight 633, DyLight 649, DyLight 680, DyLight 800, HiLyte Fluor™ 488, HiLyte Fluor™ 555, HiLyte Fluor™ 647, HiLyte Fluor 680, HiLyte Fluor™ 750), a radioisotope (e.g., ³H, ³⁵S, ¹²⁵I), a heavy atom (e.g., selenium) or a heavy isotope (e.g., ¹³C, ¹⁵N, ¹⁸O, ³⁴S, ²H). For example, amino acids or charged tRNAs that include amino acids having incorporated ¹⁸O, ¹³C, ¹⁵N, etc. can be present in an in vitro synthesis system for incorporation into proteins via the in vitro translation process to label the proteins for mass spectrometry or nuclear magnetic resonance spectroscopy. Selenium or other heavy atoms can also be incorporated into amino acids (such as, for example selenomethionine) or the amino acid portion of charged tRNAs for labeling of proteins with heavy atoms, for proteins to be analyzed, for example, using NMR spectroscopy or X-ray crystallography.

Free amino acids or the amino acid moieties of tRNAs can modified to include fluorophores can be incorporated into proteins using in vitro translation. As nonlimiting examples, amino acids can be labeled with BODIPY dyes, fluorescein isothiocyanate (FITC), fluorescamine dyes, or cyanine dyes. In some embodiments, constructs encoding a POI are engineered to contain stop codons and suppressor tRNAs charged with labeled amino acids are incorporated into proteins during in vitro translation. See, for example, Traverso et al. (2003) “Multicolor in vitro translation” in Nature Biotechnology, 21: 1093-97, and Kajihara et al. (2006) Nature Methods 3: 923-929, and Hohsaka et al. (2003) Nucleic Acids Res. Suppl. No. 3 271-272, incorporated by reference herein for all disclosure of incorporating fluorophore-conjugated amino acids into proteins. In other embodiments, initiator tRNAs are included in the in vitro translation reaction, in which the initiator tRNAs are charged with fluorophore-containing amino acids for incorporation into the translated protein. See, for example, U.S. Pat. No. 6,306,628 and U.S. Pat. No. 6,875,592, which are incorporated by reference herein in their entirety.

In yet other embodiments, a POI is translated in an IVPS system that includes a scaffold protein, or a POI is translated in an IVPS system that also synthesizes a scaffold protein, and the POI includes a sequence that can bind a fluorophore or can bind a reagent that can be conjugated to a fluorophore. The sequence can be a peptide tag, such as a lumio tag that binds tetra-arsenical or biarsenical compounds that fluorescently label the protein, or can be a streptavidin sequence for binding biotin, that can be conjugated to a fluorophore, or any other affinity tag for binding a labeled reagent.

Fluorescence assays, such as but not limited to fluorescence resonance energy transfer (FRET), time-resolved fluorescence (TRF), fluorescence polarization (FP), fluorescence recovery after photobleaching (FRAP), fluorescence activated cell sorting (FACS), fluorescence correlation spectroscopy (FCS), fluorescence microscopy, or Cary fluorescence spectrophotometery may be performed on fluorophore-labeled proteins to study ligand binding or protein-protein interactions. The fluorophore-labeled POI can include a FRET donor or acceptor, where the other member of the FRET pair is a label on another residue or region of the same POI, a label on a second POI provided in the same assay system, or a label on a lipid or partitioned with lipid that is part of the PPP that includes the POI.

In FRET, fluorescent moieties are typically chosen such that the excitation spectrum of one of the moieties (the acceptor fluorescent moiety) overlaps with the emission spectrum of the donor fluorescent moiety. The donor fluorescent moiety is excited by light of appropriate wavelength and intensity within the donor fluorescent moiety's excitation spectrum and under conditions in which direct excitation of the acceptor fluorophore is minimized. The donor fluorescent moiety then transfers the absorbed energy by non-radiative means to the acceptor, which subsequently re-emits some of the absorbed energy as fluorescence emission at a characteristic wavelength. FRET applications can include TR-FRET applications. In these embodiments, a Ln complex, such as a Eu or Tb metal chelate, is used as a fluorescent donor moiety, as described above. Typically, the Ln complex is chosen so that one of its emission bands overlaps with an excitation band of the acceptor fluorescent moiety. FRET pairs and their selection are well-known in the art.

The efficiency of FRET is dependent on the separation distance and the orientation of the donor fluorescent moiety and acceptor fluorescent moiety, the fluorescent quantum yield of the donor moiety, and the spectral overlap with the acceptor moiety. Forster derived the relationship: E=(F.degree.−F)/F.degree.=Ro.sup.6/(R.sup.6+Ro.sup.6) where E is the efficiency of FRET, F and F.degree. are the fluorescence intensities of the donor in the presence and absence of the acceptor, respectively, and R is the distance between the donor and the acceptor. Ro, the distance at which the energy transfer efficiency is 50% of maximum is given (in .ANG.) by: Ro=9.79.times.10.sup.3(K.sup.2QJn.sup.−4).sup.1/6 where K2 is an orientation factor having an average value close to 0.67 for freely mobile donors and acceptors, Q is the quantum yield of the unquenched fluorescent donor, n is the refractive index of the intervening medium, and J is the overlap integral, which expresses in quantitative terms the degree of spectral overlap. The characteristic distance Ro at which FRET is 50% efficient depends on the quantum yield of the donor, the extinction coefficient of the acceptor, the overlap between the donor's emission spectrum and the acceptor's excitation spectrum, and the orientation factor between the two fluorophores.

Labeling of a POI such as a membrane protein that is inserted into PPPs can make possible membrane protein-ligand binding studies, in which ligand binding affects the fluorescence properties of the labeled protein. In related embodiments, the ligand can also be labeled, and fluorescence detection methods such as FRET can be used to assess ligand-membrane protein binding. The present invention thus includes methods of translating a membrane protein in an IVPS system that includes PPPs, in which a label or tag that can directly or indirectly bind a label is incorporated into the translated membrane protein.

Labeling of a membrane protein that is inserted into PPPs can also make possible protein-protein interaction studies, including but not limited to membrane protein-protein interaction studies (such as but not limited to receptor dimerization studies) in which protein-protein interaction affects the fluorescence properties of the labeled protein The assays can include, but are not limited to, FRET and TRET, and include assays that monitor fluorescence quenching. One or both of the proteins can be labeled. One or both of the proteins can be synthesized as a fluorescent protein fusion protein.

Assays, including but not limited to assays of ligand binding, ion channel activity, and protein-protein interaction can be conducted on arrays, in which the arrays include PPPs with inserted MPOIs. In this way, assays on membrane proteins can be conducted in a high throughput mode, as laborious and customized purification procedures are obviated.

The present invention also includes methods of incorporating two or more different membrane proteins of interest into a common PPP using in vitro translation methodologies. In these embodiments, the different membrane proteins can be translated in a common in vitro reaction using the same or different nucleic acid template molecules. For example, multi-site GATEWAY® vectors (Invitrogen, Carlsbad, Calif.) can be used to clone at least two open reading frames in the same vector. Labels can be incorporated into the proteins during translation or the different proteins can designed with different tags that can be used for binding different labeling reagents. In this way, fluorescence measurements, such as but not limited to FRET and TRET can be used to monitor protein-protein interactions in a phospholipids bilayer, including protein-protein interactions that occur within protein complexes having multiple proteins.

FRET can be manifested as a reduction in the intensity of the fluorescent signal from the donor, reduction in the lifetime of its excited state, and/or an increase in emission of fluorescence from the acceptor fluorescent moiety. For example, when a membrane POI having a donor fluorescent moiety and, for example, a lipid having an acceptor fluorescent moiety are within the required distance, FRET is observed. When the donor fluorescent moiety and the acceptor fluorescent moiety physically separate, FRET is diminished or eliminated. Under these circumstances, fluorescence emission from the donor increases and fluorescence emission from the acceptor decreases. Accordingly, a ratio of emission amplitudes at wavelengths characteristic (e.g., the emission maximum) of the donor relative to the acceptor should increase as compared to the same ratio under FRET conditions (e.g., when emission of the donor is quenched (e.g., reduced) by the acceptor).

Changes in the degree of FRET can be determined as a function of a change in a ratio of the amount of fluorescence from the donor and acceptor moieties, a process referred to as “ratioing.” By calculating a ratio, the assay is less sensitive to, for example, well-to-well fluctuations in substrate concentration, photobleaching and excitation intensity, thus making the assay more robust. This is of particular importance in automated screening applications where the quality of the data produced is important for its subsequent analysis and interpretation.

For example, in some embodiments of the method, a ratiometric analysis is performed, wherein a ratio of fluorescence emission at two different wavelengths is compared between a protease mixture and a control mixture. In a typical FRET-based assay, the two wavelengths can correspond to the emissions maxima for two detectable (e.g., fluorescent) moieties of the composition. Thus, if a receptor protein comprises a label that is a member of a FRET pair, the receptor bound by a ligand may have a different conformation than when not bound, and thus a different distance from its FRET partner (the ligand itself, a lipid in the PPP, or another protein present in the PPP). Accordingly, in a ligand-bound state, for example, the receptor may maintain FRET between the donor and acceptor moieties (e.g., the FRET pair), resulting in a low emissions ratio of the donor to the acceptor moiety. An unbound receptor will display (in this example) reduced FRET between the donor and acceptor moieties, leading to a larger emissions ratio of the donor to the acceptor moiety. In some embodiments, the emissions ratio of the “no ligand” control sample will be more than 1.5, 2, 3, 4, 5, 7, 10, 15, 20, 25, 30, 40, 50, or 100 times larger than the emissions ratio of a sample with a high affinity ligand. This example is for illustrative purposes only, as assay formats can vary widely.

Fluorescent labels can be incorporated into PPPs by partitioning into the lipid bilayer or by use of fluorphore-conjugated lipids in making the PPPs. For example, classes of lipophilic dyes that associate with lipids within bilayers are provided in the Molecular Probes Handbook, 10^{th} edition, herein incorporated by reference in its entirety. Lipids can also be labeled by conjugating any of a variety of fluorophores. Fluorescence changes due to conformational changes in a membrane protein in PPPs can be monitored, providing an assay for membrane protein function. In embodiments in which the membrane protein is labeled with a first fluorophore and one or more lipids is labeled with a second fluorophore, and FRET can occur between the first and second fluorophores, FRET or TRET based assays can be used to monitor protein function, such as ligand binding, which affects protein conformation.

A FRET pair includes a fluorophore donor and a fluorophore acceptor, in which the emission wavelength spectrum of the fluorphore donor overlaps the absorption wavelength spectrum of the fluorophore acceptor. Radiationless energy transfer leading to fluorescence at the acceptor wavelength occurs when the FRET partners are within a certain distance of one another, in most cases within 1-10 nm of one another.

Fluorescent labels can be incorporated into PPPs by partitioning into the lipid bilayer or by use of fluorphore-conjugated lipids in making the PPPs. For example, classes of lipophilic dyes that associate with lipids within bilayers include are provided in the Molecular Probes Handbook, 10^{th} edition (Chapter 13). For example, fatty acids labeled with BODIPY fluorophores (BODIPY 503/512, BODIPY 500/510, BODIPY 530/550, BODIPY 558/568, BODIPY 581/591), nitrobenzodiazole (NBD), and pyrene, as well as dansyl undecanoic acid (DAUSA) and cis-parinaric acid are available from Molecular Probes (Eugene Oreg.). Phospoholipids can also be labeled with BODIPY dyes; for example, BODIPY FL dye-labeled phosphtidic acid, BODIPY 530/550-labeled glycerophophocholine, and BODIPY 581/591-labeled glycerophosphocholine are all commercially available. The phospholipid analog beta-DPH HPC and derivatives as well as phospholipids with NBD-labeled acyl chains and purene-labeled acyl chains can also be incorporated into PPPs used in the methods and compositions of the invention. The head groups of a phospholipid can be labeled, for example, with NBD, fluorescein, Oregon green 488, BODIPY FL, rhodamine, Texas red, maleimide, dansyl, marina blue dye, pacific blue dye, or bioin, which can be conjugated to a dye. Sphingolipids for incorporation into PPPs can be labeled, for example, with BODIPY dyes or NBD, as can steroids, such as cholesteryl esters and cholesterol analogs. Lipopolysaccharides can be labeled with BODIPY or Alexa Fluor dyes for incorporation into PPPs. All of these conjugates are commercially available from Molecular Probes (Eugene, Oreg.).

Other labels, such as fluorophores, can be amphiphilic molecules having a charged fluorophore group that orients external to the membrane, and a hydrophobic tail that inserts into membranes. For example, dialkylcarbocyanine probes (e.g., DiI (e.g., 1,1′-dioctadecyl-3,3,3′,3′-tetramethylindocarbocyanine perchlorate; DiIC₁₈(3); e.g., Invitrogen Catalog Number D-282); DiO (e.g., 3,3′-dioctadecyloxacarbocyanine perchlorate; DiOC₁₈(3); e.g., Invitrogen Catalog Number—D-275); DiD (e.g., 1,1′-dioctadecyl-3,3,3′,3′-tetramethylindodicarbocyanine, 4-chlorobenzenesulfonate salt; DiIC₁₈(5); e.g., Invitrogen Catalog Number D-7757); DiR (e.g., 1,1′-dioctadecyl-3,3,3′,3′-tetramethylindotricarbocyanine iodide; DiTC₁₈(7); e.g., Invitrogen Catalog Number D-12731) and analogs thereof) can be incorporated into PPPs and used, for example, for detecting PPPs as well as for FRET studies. Other amphilic or nonpolar dyes that can be used in membrane labeling include, for example, amphiphilic derivatives of rhodamines, fluoresceins, and coumarins, for example, octadecyl rhodamine B, 5-dodecanoyl-aminofluorescein, 5-hexadecanoyl-fluorescein, 5-octadecanolyl-aminofluorescein, and 4-heptadecyl-7-hydroxycoumarin. Diphenylhexatriene (DPH), Trimethylammonium DPH, Trimethylammonium phosphate DPH, DPH propionic acid, and nonpolar BODIPY fluorophores are yet other lipid-partitioning fluorescent molecules. Nonpolar pyrenes, Nile red, bimane azide, prodan, laurdan, dapoxyl derivatives, anilinonaphthalenesulfonate (ANS), bis ANS, DCVJ, and 4-amino-4′-benzamidostilbene-2,2′-disulfonic acid are additional lipophilic molecules that can be used to label PPPs. All of the aforementioned fluorophore-labeled molecules are described in Haugland et al., and are available from Molecular Probes (Eugene, Oreg.).

Fluorescence changes due to conformational changes in a membrane protein in PPPs can be monitored, providing an assay for membrane protein function. In embodiments in which the membrane protein is labeled with a first fluorophore and one or more lipids is labeled with a second fluorophore, and FRET can occur between the first and second fluorophores, FRET or TRET based assays can be used to monitor protein function, such as ligand binding, which affects protein conformation.

PPPs having incorporated lipophilic dyes can be used for tissue or in vivo imaging, in which the PPPs include a POI that can target the PPP to a tissue, cell type, organ, etc. For example, a transmembrane protein inserted into PPPs can be fused to an antibody or portion thereof that recognizes a molecule expressed on cells or pathogens to be detected. Lipophilic fluorescent dyes include, but are not limited to, DiA, DiO, DiD, DiR, and DiI. CT contrast reagents such as iodinated or brominated fatty acids or cholesterol can also be inserted into PPPs in the self-assembly process. Drug delivery can also be effected by compound-loaded PPPs.

Fluorescence assays such as FRET and TRET assays are contemplated for PPPs that include membrane proteins of interest that are made using IVPS systems as well as manufactured using one or more membrane proteins that are not synthesized in an IVPS systems.

### Other Moieties

One or more other moieties or binding agents with in vitro or in vivo activity or utility may also be incorporated into PPPs, either with or without a POI. Active moieties or binding agents include, for example, polypeptides, peptides, protein- or peptide-nucleic acids (PNAs), antibodies, peptibodies, or derivatives or fragments thereof. Antibodies include whole antibodies, a human Fc region, fully human, antibodies, humanized antibodies, chimeric antibodies, CDR grafted antibodies, single chain variable fragments of a specific antibody, single chain Fv fragments of the a specific antibody, such as heavy chain variable regions of the antibody, light chain variable regions, Fab fragments of the antibody and other antibody fragments having specific binding activity to an antigen. A peptibody which refers to a molecule comprising an antibody Fc domain attached to at least one peptide (e.g., as described by PCT publication WO 00/24782, published May 4, 2000, which is incorporated herein by reference in its entirety). Other active moieties include, for example, cytotoxic drugs or active fragments thereof, diphtheria A chain, exotoxin A chain, ricin A chain, abrin A chain, curcin, crotin, phenomycin, enomycin, radiochemicals, and the like. Any of these moieties or binding agents may be used to target PPPs to particular cells or tissues in vitro or in vivo. In certain embodiments, such PPPs may also be used in therapeutic or other settings.

### Scaffold Protein—POI Compositions

The present invention provides, in another embodiment, a composition that includes one or more membrane proteins associated with one or more scaffold proteins. Typically, the composition is a soluble, isolated complex of one or more scaffold proteins and one or more membrane proteins in an aqueous solution. The complex can include a lipid, such as a phospholipid. The complex of a membrane protein and a scaffold protein can, in some embodiments, be substantially lipid-free. The membrane protein of the complex is typically synthesized using an IVPS system, as disclosed herein, typically in the presence of the scaffold protein. A complex in illustrative examples of this embodiment of the invention can be free of detergents. The complex can also be a cell-free complex that includes a scaffold protein, all or a portion of a membrane protein, typically at least the N-terminus portion, one or more ribosomes, and one or more RNA molecules, such as an RNA molecule encoding the membrane protein. The complex can include lipid or be substantially free of lipid. The complex can be an isolated complex. The complex can be optionally bound to a solid support via a nucleic acid template encoding either the scaffold protein or the POI, or via the scaffold protein or POI, either of which can optionally comprise a peptide tag.

In certain embodiments, isolated PPPs comprising one or more scaffold proteins, optionally one or more phospholipids, and one or more dyes are provided. The scaffold protein may be as described herein, and is preferably a recombinant scaffold protein. The dye is preferably a fluorophore such as a an amphiphilic molecule having a charged fluorophore group that orients external to the membrane, and a hydrophobic tail that inserts into membranes. In certain embodiments, the dye is a dialkylcarbocyanine probe such as DiI, DiO, DiD, DiR, or an analog thereof. In other embodiments, the may be an amphilic or nonpolar dye. Preferred dyes include, for example, and without limitation, amphiphilic derivative of rhodamine, fluorescein, or coumarin such as octadecyl rhodamine B, 5-dodecanoyl-aminofluorescein, 5-hexadecanoyl-fluorescein, 5-octadecanolyl-aminofluorescein, and 4-heptadecyl-7-hydroxycoumarin. Diphenylhexatriene (DPH), Trimethylammonium DPH, Trimethylammonium phosphate DPH, DPH propionic acid, or a nonpolar BODIPY fluorophore. In some embodiments, the dye is a lipid-partitioning fluorescent molecule. In others, the dye is a nonpolar pyrenes, Nile red, bimane azide, prodan, laurdan, dapoxyl derivatives, anilinonaphthalenesulfonate (ANS), bis ANS, DCVJ, or 4-amino-4′-benzamidostilbene-2,2′-disulfonic acid.

Isolated PPPs comprising a scaffold protein, optionally a phospholipid, one or more dyes, and a POI are also provided. In some embodiments, the POI is a membrane protein. Also provided are PPPs comprising a scaffold protein, optionally a phospholipid, one or more dyes, optionally one or more POIs, and one or more fluorescent proteins such as GFP, EGFP, BFP, CFP, RFP, or YFP or fluorescent variants thereof with at least 80% sequence identity thereto.

Composition comprising an isolated PPP comprising one or more scaffold proteins, optionally one or more phospholipids, one or more dyes and a cell extract. The PPPs may be as described herein and the cell extract may be of any origin including but not limited to prokaryotic, eukaryotic and/or synthetic. Prokaryotic cell extracts include those of bacteria such as E. coli. Eukaryotic extracts include but are not limited to those of mammalian cells, such as rabbit reticulocytes or plants, such as a wheat germ extract.

Composition comprising an isolated PPP comprising one or more scaffold proteins, optionally one or more phospholipids, optionally one or more dyes, optionally a cell extract, and a ligand are also provided. In certain embodiments, the PPP may further comprise a POI to which the ligand associates. In some embodiments, the ligand is associated with the POI prior the incorporation of the POI into the PPP. In others, the ligand is associated with the POI after incorporation of the POI into the PPP. The PPPs and cell extracts may be as described herein. Non-limiting illustrative examples of such compositions are described in the Examples, such as the association of EmrE and bacteriorhodopsin with their respective ligands.

In certain embodiments, compositions of the present invention may be administered to a host (e.g., a human being) using any of a variety of techniques known to those of skill in the art. The composition(s) may be processed in accordance with conventional methods of pharmacy to produce medicinal agents for administration to patients, including humans and other mammals (i.e., a “pharmaceutical composition”). The pharmaceutical composition is preferably made in the form of a dosage unit containing a given amount of POI or PPP, for example. A suitable daily dose for a human or other mammal may vary widely depending on the condition of the patient and other factors, but may be determined using routine methods.

The pharmaceutical composition may be administered orally, parentally, by inhalation spray, rectally, intranodally, or topically in dosage unit formulations containing conventional pharmaceutically acceptable carriers, adjuvants, and vehicles. The term “pharmaceutically acceptable carrier” or “physiologically acceptable carrier” as used herein refers to one or more formulation materials suitable for accomplishing or enhancing the delivery of the pharmaceutical composition. A “pharmaceutical composition” is a composition comprising a therapeutically effective amount of a nucleic acid or polypeptide. The terms “effective amount” and “therapeutically effective amount” each refer to the amount of a composition used to induce or enhance an effective response or to provide for its use as an imaging agent.

For oral administration, the pharmaceutical composition may be of any of several forms including, for example, a capsule, a tablet, a suspension, or liquid, among others. Liquids may be administered by injection as a composition with suitable carriers including saline, dextrose, or water. The term parenteral as used herein includes subcutaneous, intravenous, intramuscular, intrasternal, infusion, or intraperitoneal administration. Suppositories for rectal administration of the drug can be prepared by mixing the drug with a suitable non-irritating excipient such as cocoa butter and polyethylene glycols that are solid at ordinary temperatures but liquid at the rectal temperature.

The dosage regimen for immunizing a host or otherwise treating a disorder or a disease with a composition of this invention is based on a variety of factors, including the type of disease, the age, weight, sex, medical condition of the patient, the severity of the condition to be treated, the type of imaging procedure being performed, the route of administration, and/or the particular composition being employed.

### In Vivo Imaging

The PPPs and compositions comprising PPPs described herein may be utilized for in vivo or medical imaging. The PPPs will typically include a detectable label. PPPs may be used in magnetic resonance imaging (MRI), nuclear medicine, positron emission tomography, projection radiography, photoacoustic imaging, and various types of tomography (positron emission, linear, poly tomography, zonography, orthopantomography, computed tomography). PPPs containing detectable labels may be administered to a host to visualize particular cells or tissues. PPPs containing binding agents such as ligands or antibodies may also be targeted to particular cells and/or tissues to assist in the diagnosis and/or treatment of diseases such as cancer. For instance, a detectably labeled PPP may also include an antibody (or reactive portion thereof) with reactivity against a prostate cancer antigen. The detectably labeled PPP may be administered to a host and detected in the host to determine the where in the body prostate cancer cells are present. Other similar embodiments would be understood by one of skill in the art and are contemplated herein.

### Kits

Also provided are kits for IVPS including a cell extract for in vitro translation that includes at least one scaffold protein such as an apolipoprotein or AAHC protein as described above. The scaffold protein provided in the cell extract can be bound to lipid, such as phospholipid, such as in a phospholipid-protein particle, or in other embodiments, not bound to lipid. In some embodiments, a kit includes a cell extract for in vitro translation that includes a PPP, and at least one of a buffer, a salt, an enzyme, a chemical energy source, amino acids, a tRNA, an inhibitor, a label, a detergent, and a surfactant. In certain embodiments, components of the kit are affixed to a solid support such as a bead or multi-well plate. In certain embodiments, the PPP or POI, or PPP including a POI, are arranged in arrayed format for high-throughput screening.

The invention also includes kits that include a scaffold protein, and a cell extract that are provided in separate containers. The scaffold protein provided in the cell extract can be bound to lipid, such as phospholipid, such as in a phospholipid-protein particle, or in other embodiments, not bound to lipid. A scaffold protein can be any disclosed herein or available to one of skill in the art. A kit can include more than one scaffold protein. A PPP can be any disclosed herein. The kits can also include, provided in the cell extract or separately, a chemical energy source, and one or more amino acids. The kit can also include one or more buffers, one or more salts, one or more enzymes, one or more cofactors, one or more inhibitors, one or more labels, one or more lipids, or one or more surfactants, any or all of which can be provided in the cell extract, or separate from the cell extract.

The kits may include nucleic acid templates encoding one or more scaffold proteins and/or one or more POIs. The nucleic acid template or templates may consist of any type of nucleic acid, such as DNA or RNA. The POI and scaffold proteins may be encoded by one or more nucleic acid templates. Where multiple templates are utilized, the templates may be different types of nucleic acids. For example, where two templates are utilized, one may be DNA and one may be RNA, or both may be either DNA or RNA. The nucleic acid templates encoding the POI and scaffold protein may be the same or different. A single nucleic acid template encoding both the POI and the scaffold protein may include separate promoters controlling expression of the POI and the scaffold protein, and/or may include a common promoter along with another element, such as an IRES sequence inserted between the two gene sequences, allowing for expression of both proteins from the same promoter. The kit can also include one or more vectors including one or more nucleic acid templates. Suitable vectors are described herein and are known in the art.

In some embodiments, the kit includes a cell extract and a nucleic acid template that encodes a scaffold protein. A scaffold protein can be any disclosed herein or otherwise available to one of skill in the art. The cell extract of the kit can include one or more lipids, such as one or more phospholipids, or the cell extract can be essentially free of phospholipids. The nucleic acid template encoding a scaffold protein can be provided in the cell extract or separately. The kit can also include one or more buffers, one or more salts, one or more enzymes, one or more cofactors, one or more inhibitors, one or more labels, one or more lipids (e.g., phospholipids), or one or more surfactants, any or all of which can be provided in the cell extract, or separate from the cell extract.

In certain embodiments, the kit comprises a cell extract, a ligand, and an isolated PPP comprising a scaffold protein and one or more phospholipids. In certain embodiments, the kit also includes a POI. In some embodiments, the kit comprises an isolated cell extract (e.g., in a container such as a tube) and an isolated PPP. The PPP may also optionally include a dye or other tag as described herein. Thus, in certain embodiments, the kit contains a cell extract in one container and an isolated PPP in another container. In others, the kit contains a cell extract in one container and an isolated PPP and a dye or other tag in another container. In still others, the components are packaged within the same container. Where a POI is also part of the kit, it may be as an isolated protein or as a nucleic acid template encoding the POI. The POI may be included in a separate container, or in any of the other containers of the kit. In preferred embodiments, the kit would include separate containers for the cell extract, the PPP, and the POI whether in protein or nucleic acid template form. The contents of these containers may then be combined as needed to carry out the methods described herein.

The kits may include any useful components described herein or elsewhere, including but not limited to affinity tags, labels, reagents and systems for isolating PPPs (whether labeled or not labeled), buffers, enzymes, additional proteins or nucleic acid templates, and the like. These additional components may be provided in the same containers or in different containers, depending on the particular application. The kits may also include instructions for use.

### Services

In certain embodiments, a commercial service for performing a method and/or that uses a composition described herein is provided. Any of the methods provided herein can be sold as a commercial service. For example, the commercial service can include an offer for consideration and/or payment of consideration for performing a method that includes a drug screening method performed by contacting an isolated PPP comprising a target protein or POI such as EmrE (SEQ ID NO: 43), bacteriorhodopsin (SEQ ID NO: 44), a polypeptide expressible from the Invitrogen Ultimate™ ORF clone collection, a G protein-coupled receptor (GPCR), G protein-coupled receptor family C group 5 member C (SEQ ID NO: 45), G protein-coupled receptor 157 (SEQ ID NO: 46), serotonin receptor HTR1 (SEQ ID NO: 47), endothelin receptor type B (SEQ ID NO: 48), opiate receptor-like 1 (SEQ ID NO: 49), cholinergic receptor muscarinic 2 (SEQ ID NO: 50), histamine receptor H2 (SEQ ID NO: 51), dopamine receptor D1 (SEQ ID NO: 52), melanocortin 5 receptor (SEQ ID NO: 53), corticotropin releasing hormone receptor 1 (SEQ ID NO: 54), 5-hydroxytryptamine (serotonin) receptor 1A (SEQ ID NO: 55), cholinergic receptor muscarinic 1 (SEQ ID NO: 56), CD24 (SEQ ID NO: 57), glycophorin E (SEQ ID NO: 58), glycophorin B (SEQ ID NO: 59), chemokine-like factor (SEQ ID NO: 60), glycophorin A (SEQ ID NO: 61), murine microsomal glutathione S-transferase 1 (SEQ ID NO: 62), phosphatidylinositol glycan anchor biosynthesis class P (SEQ ID NO: 63), epiregulin (SEQ ID NO: 64), epiregulin (SEQ ID NO: 65), CD99 (SEQ ID NO: 66), murine Mpv17 transgene (SEQ ID NO: 67), MpV17 mitochondrial inner membrane protein (SEQ ID NO: 68), translocase of inner mitochondrial membrane 22 homolog (SEQ ID NO: 69), ninjurin 2 (SEQ ID NO: 70), signal peptide peptidase-like 2B (SEQ ID NO: 71), CKLF-like MARVEL transmembrane domain containing 1 (SEQ ID NO: 72), golgi transport 1 homolog B (SEQ ID NO: 73), leukotriene C4 synthase (SEQ ID NO: 74), angiotensin II receptor-associated protein (SEQ ID NO: 75), arachidonate 5-lipoxygenase-activating protein (SEQ ID NO: 76), signal peptide peptidase 3 (SEQ ID NO: 77), leptin receptor (SEQ ID NO: 78), microsomal glutathione S-transferase 3 (SEQ ID NO: 79), dystrobrevin binding protein 1 (SEQ ID NO: 80), PRA1 domain family member 2 (SEQ ID NO: 81), phosphatidic acid phosphatase type 2 domain containing 1B (SEQ ID NO: 82), and human adrenomedullin receptor protein (SEQ ID NO: 83), or other target protein or POI known to those of skill in the art with a test compound and detecting a change in the target protein.

In another embodiment, the commercial service can be a protein expression service, for expressing a protein selected from the group consisting of EmrE (SEQ ID NO: 43), bacteriorhodopsin (SEQ ID NO: 44), a polypeptide expressible from the Invitrogen Ultimate™ ORF clone collection, a G protein-coupled receptor (GPCR), G protein-coupled receptor family C group 5 member C (SEQ ID NO: 45), G protein-coupled receptor 157 (SEQ ID NO: 46), serotonin receptor HTR1 (SEQ ID NO: 47), endothelin receptor type B (SEQ ID NO: 48), opiate receptor-like 1 (SEQ ID NO: 49), cholinergic receptor muscarinic 2 (SEQ ID NO: 50), histamine receptor H2 (SEQ ID NO: 51), dopamine receptor D1 (SEQ ID NO: 52), melanocortin 5 receptor (SEQ ID NO: 53), corticotropin releasing hormone receptor 1 (SEQ ID NO: 54), 5-hydroxytryptamine (serotonin) receptor 1A (SEQ ID NO: 55), cholinergic receptor muscarinic 1 (SEQ ID NO: 56), CD24 (SEQ ID NO: 57), glycophorin E (SEQ ID NO: 58), glycophorin B (SEQ ID NO: 59), chemokine-like factor (SEQ ID NO: 60), glycophorin A (SEQ ID NO: 61), murine microsomal glutathione S-transferase 1 (SEQ ID NO: 62), phosphatidylinositol glycan anchor biosynthesis class P (SEQ ID NO: 63), epiregulin (SEQ ID NO: 64), epiregulin (SEQ ID NO: 65), CD99 (SEQ ID NO: 66), murine Mpv17 transgene (SEQ ID NO: 67), MpV17 mitochondrial inner membrane protein (SEQ ID NO: 68), translocase of inner mitochondrial membrane 22 homolog (SEQ ID NO: 69), ninjurin 2 (SEQ ID NO: 70), signal peptide peptidase-like 2B (SEQ ID NO: 71), CKLF-like MARVEL transmembrane domain containing 1 (SEQ ID NO: 72), golgi transport 1 homolog B (SEQ ID NO: 73), leukotriene C4 synthase (SEQ ID NO: 74), angiotensin II receptor-associated protein (SEQ ID NO: 75), arachidonate 5-lipoxygenase-activating protein (SEQ ID NO: 76), signal peptide peptidase 3 (SEQ ID NO: 77), leptin receptor (SEQ ID NO: 78), microsomal glutathione S-transferase 3 (SEQ ID NO: 79), dystrobrevin binding protein 1 (SEQ ID NO: 80), PRA1 domain family member 2 (SEQ ID NO: 81), phosphatidic acid phosphatase type 2 domain containing 1B (SEQ ID NO: 82), and human adrenomedullin receptor protein (SEQ ID NO: 83), or other target protein or POI known to those of skill in the art, wherein the protein is produced within a PPP comprising the protein. In illustrative embodiments, the protein is produced using in vitro translation.

The following examples are intended to illustrate but not limit the invention.

### EXAMPLE 1

### Manufacture of Nanolipoprotein Particles from Apolipoprotein and Phospholipid

Nanolipoprotein particles (PPPs) were made using the mature, processed form of Apolipoprotein A1, dimyristoyl phosphatidyl choline (DMPC), and cholate. The Apo A1 protein was synthesized in cultured E. coli cells (BL21 DE3*) that contained a construct that included a pEXP5-NT vector containing a his tag sequence (Invitrogen, Carlsbad, Calif.) and an engineered Apo A1 sequence from Invitrogen ULTIMATE™ ORF clone IOH7318 having the protein encoding sequence of Genbank gi 4557320 (NM_{—}00039.1). The sequence was deleted at the five prime end to create a sequence in the plasmid construct that encoded the mature, N-terminally processed form of the human Apo A1 gene. The protein, lipid, and detergent components were incubated to form phospholipid-apolipoprotein particles in a self assembly process, after which the cholate detergent was removed by absorption to Bio-Beads SM-2 (Bio-Rad, cat #152-3920).

A DMPC5 ml stock solution of 400 mM DMPC was prepared in 800 mM Cholate, 10 mM Tris, pH 8, 150 mM NaCl, 0.25 mM EDTA, 0.01% sodium azide. Briefly, DMPC powder was added to the cholate solution and vortexed in a glass screw cap tube. The DMPC was dissolved by using a cycle of 30° C. water bath incubation and waterbath sonication followed by gentle mixing at room temperature for 1 hour or until solubilzed. The final solution was sealed under nitrogen and stored at room temperature until use. Apo A1 protein (10 mg of mature-form human Apo A1 that included a his tag, purified by affinity chromatography using Ni-NTA, at 8.54 mg/mL) was added to a glass screw cap bottles containing various amounts of the DMPC/Cholate stock solution. Three molar ratios of [DMPC;ApoA:Cholate] were investigated. The different molar ratios were (a) 70:1:140, (b) 140:1:280 and (c) 280:1:560 in a final volume of 2.0 mL. The mixtures were incubated in a 30° C. water bath for 10 minutes then at room temperature for 10 min, with light mixing between temperature shifts. The incubation process was repeated two more times. The PPP mixture was then incubated at room temperature for 90 minutes. Cholate was removed with the addition Bio-Beads SM-2 resin (added a minimum of 0.345 grams of beads per gram of cholate) The mixture was mixed (end over end on a rotating mixer) for 90 minutes at room temperature. The crude PPPs were 0.2 um filtered through a PVDF syringe filter to remove the Bio-Beads.

To test for synthesis of a membrane protein in soluble form in an in vitro system that included the manufactured PPPs, bacteriorhodopsin from Halobacterium halobium was transcribed and translated from the pIVEX2.4b in the EXPRESSWAY™ coupled in vitro transcription/translation system (Invitrogen, Carlsbad, Calif.) that includes an E. coli cell extract. Six microliters of PPP self-assembly preparations that included 5 mg/mL Apo A1 protein PPPs were added to 100 microliters of EXPRESSWAY™ in vitro translation reaction. As controls, six microliters of 5 mg/mL or 27 mg/mL of purified “nanodiscs” that included the MSP1 protein (U.S. Pat. No. 7,048,949; amount of nanodiscs determined by MSP1 concentration) or 5 mg/mL Apo A1 protein were added to in vitro translation reactions were performed alongside the in vitro translation reactions performed with Apo A1-DMPC crude preparations. The IVPS reactions also included 10 mM retinal, the light absorbing ligand for bacteriorhodopsin that, when inserted appropriately into the bacteriorhodopsin protein, imparts a purple color to the protein. In a control reaction, retinal was omitted.

The in vitro transcription/translation reaction was performed according to the manufacturer's instruction for the EXPRESSWAY™ Maxi protein synthesis system (Invitrogen, Carlsbad, Calif.) (without the use of radiolabeled methionine) using 2.8 micrograms of the pIVEX2.4b construct that encoded the full-length bacteriorhodopsin gene as a template. The reaction was incubated for 3 hrs at 37 degrees C., with a 50 microliter feed buffer added 30 minutes into the incubation. Following incubation, one microliter aliquots of the reactions were removed from the reactions and either loaded directly on SDS PAGE gels (total, or “whole” reaction aliquots) or spun ten minutes to remove insoluble protein before loading on the gel (soluble fraction aliquots).

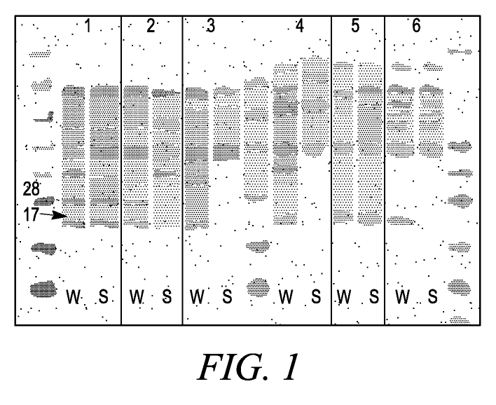

The results of gel electrophoresis are shown in FIG. 1, in which soluble bacteriorhodopsin was synthesized in an IVPS system that included PPPs made using Apolipoprotein A1 and phospholipid. “W” indicates an aliquot of the whole protein synthesis reaction (not separated into soluble and insoluble fraction); “S” indicates an equal aliquot of the soluble fraction of the reaction. Lanes 2 and 3 are the whole and soluble fractions of reactions that included 5 mg/mL PPPs made with a 70:1 ratio of DMPC to ApoA1; Lanes 4 and 5 are the whole and soluble fractions of reactions that included 5 mg/mL PPPs made with a 140:1 ratio of DMPC to ApoA1; and lanes 6 and 7 are the whole and soluble fractions of reactions that included 5 mg/mL PPPs made with a 140:1 ratio of DMPC to ApoA1. Lanes 13 and 14 are the whole and soluble fractions of reactions that included 5 mg/mL of Apo A1 protein but did not include PPPs. Comparison of lanes 2 and 3 with lanes 13 and 14 demonstrates that PPPs result in a majority of the synthesized protein being made in soluble form, and a greater amount of bacteriorhodopsin is synthesized in soluble form in the presence of PPPs (lane 3) than in the absence of PPPs (lane 14).

In vitro translation reactions that included retinal and PPPs were visibly purple in color after the in vitro synthesis reaction, indicating that the bacteriorhodopsin had been synthesized in its active conformation. Reactions that included retinal but no PPPs were yellowish, whereas in the absence of both retinal and PPPs, the IVPS reactions were colorless after incubation.

### EXAMPLE 2

### Co-Translation of a Scaffold Protein and a Membrane Protein in the Presence of Phospholipid Produces Active Soluble Membrane Protein

In separate experiments, and bacteriorhodopsin, a seven transmembrane domain membrane protein, was synthesized in vitro in a reaction in which the MSP1 membrane scaffold protein was also synthesized. In control reactions, bacteriorhodopsin and MSP1 were synthesized separately in the in vitro synthesis system. The EXPRESSWAY™ coupled in vitro transcription/translation system (Invitrogen, Carlsbad, Calif.) was used to produce MSP1 from the a pIVEX2.4b vector that included the MSP1 gene and bacteriorhodopsin from a pIVEX2.4b vector.

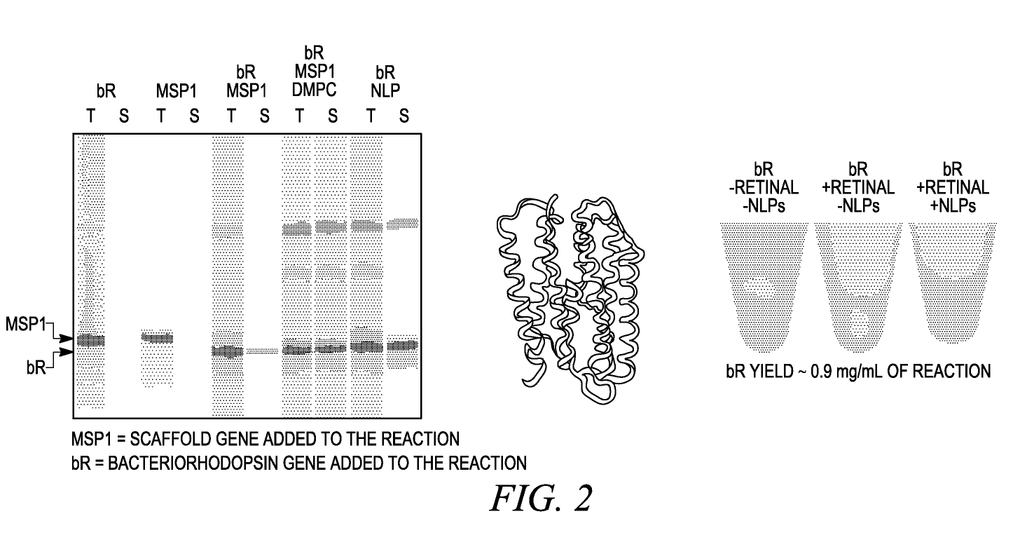

One microgram of each template was added to 100 microliter reactions in which DMPC liposomes were either present (30 micrograms) or not present. In a control reaction, pre-made purified “nanodiscs” that included DMPC and the MSP1 protein were included in the protein synthesis reactions. ³⁵S labeled methionine was included in the reactions for labeling of in vitro synthesized proteins. The reactions were set up and incubated for 3 hours at 37 degrees C. according to the manufacturer's instructions. After incubation, an aliquot of the total unfractionated reaction was removed for electrophoresis, and the incubated reactions were spun 10 min at 12,000×g, and an aliquot of the supernatant was removed to provide a soluble fraction. The aliquots were electrophoresed on SDS PAGE gels and autoradiographed. FIG. 2 shows that bacteriorhodopsin is synthesized in the absence of MSP1 in the in vitro synthesis system, but only in insoluble form (Lane 1). Scaffold protein MSP1 is also synthesized in the in vitro synthesis system, but the vast majority of the synthesized MSP1 is insoluble (Lane 3 versus Lane 4). Cotranslation of bacteriorhodopsin and MSP1 in the same reaction results in the synthesis of both proteins, but the vast majority of both synthesized proteins is in insoluble form (Lanes 5 and 6). In the presence of 30 ug of DMPC, however, both proteins are synthesized, and the majority of the synthesized protein is in soluble form (Lanes 7 and 8). As a control, bacteriorhodopsin synthesized in vitro in the presence of pre-formed, purified PPPs (that include MSP1 and DMPC) is found to be synthesized in soluble form (Lanes 9 and 10).

### EXAMPLE 3

### In Vitro Synthesis of Membrane Proteins with PPPs

To demonstrate the wide range of membrane proteins that can be translated in soluble form when PPPs are present in the reaction, different human membrane proteins were synthesized using an IVPS system that included PPPs that included the MSP1 membrane scaffold proteins and 1-palmitoyl-2-oleoyl-phosphatidyl choline (POPC). Clones from the Invitrogen Ultimate™ ORF clone collection (Invitrogen, Carlsbad, Calif.; Invitrogen.com; searchable clone collection provided at orf.invitrogen.com/cgi-bin/ORF_Browser) were used to express membrane proteins in the EXPRESSWAY™ in vitro protein synthesis system to which 100 ug of PPPs that included the MSP1 scaffold protein and POPC. Clones used for expression of GPCR proteins included: IOH14234, endothelin receptor type B (EDNRB) (NM_{—}000115.1; SEQ ID NO: 48); IOH 27433, opiate receptor-like 1 (NM_{—}000913.3; SEQ ID NO: 49); IOH28351 cholinergic receptor muscarinic 2 (NM_{—}000739.2; SEQ ID NO: 50); IOH28904, histamine receptor H2 (BC054510.2; SEQ ID NO: 51); IOH29556, dopamine receptor D1 (NM_{—}000794.3; SEQ ID NO: 52); IOH29738, melanocortin 5 receptor (NM_{—}005913.1; SEQ ID NO: 53); IOH39398, corticotropin releasing hormone receptor 1 (NM_{—}004382.2; SEQ ID NO: 54); IOH46452, 5-hydroxytryptamine (serotonin) receptor 1A (NM_{—}000524.2; SEQ ID NO: 55); and IOH56940, cholinergic receptor muscarinic 1 (NM_{—}000738.2; SEQ ID NO: 56). Clones used for expression of other membrane proteins included: IOH5911, CD24 molecule (NM_{—}013230.2; SEQ ID NO: 57); IOH12322, glycophorin E (BC017864.1; SEQ ID NO: 58); IOH58935, glycophorin B (NM_{—}002100.3; SEQ ID NO: 59); IOH58583, chemokine-like factor (NM_{—}181640.1; SEQ ID NO: 60); IOH5520, G protein-coupled receptor, family C, group 5, member C (NM_{—}004925.1; SEQ ID NO: 45); IOH7353, glycophorin A (BC005319.1; SEQ ID NO: 61); IOM19680, microsomal glutathione S-transferase 1 (mouse) (BC009155.1; SEQ ID NO: 62); IOH44755 phosphatidylinositol glycan anchor biosynthesis, class P (NM 153681.2; SEQ ID NO: 63); IOM14930, epiregulin (NM_{—}007950.1; SEQ ID NO: 64); IOH5089, CD99 molecule (NM_{—}002414.3; SEQ ID NO: 66); IOH42289, IOH58999, epiregulin (NM_{—}001432.1; SEQ ID NO: 65); IOM15042, Mpv17 transgene (mouse) (NM_{—}008622.1; SEQ ID NO: 67); IOH3860, MpV17 mitochondrial inner membrane protein (NM_{—}002437.4; SEQ ID NO: 68); IOH3712, translocase of inner mitochondrial membrane 22 homolog (NM_{—}013337.2; SEQ ID NO: 69); IOH43470, ninjurin 2 (NM_{—}016533.4; SEQ ID NO: 70); IOH4396, signal peptide peptidase-like 2B (BC001788.1; SEQ ID NO: 71); IOH58697, CKLF-like MARVEL transmembrane domain containing 1 (NM_{—}181268.1; SEQ ID NO: 72); IOH10546, golgi transport 1 homolog B (NM_{—}016072.2; SEQ ID NO: 73); IOH54642, leukotriene C4 synthase (NM_{—}145867.1; SEQ ID NO: 74); IOH 14721, angiotensin II receptor-associated protein (NM_{—}001040194.1; SEQ ID NO: 75); IOH12197, G protein-coupled receptor 157 (BC018691.1; SEQ ID NO: 46); IOH11710, arachidonate 5-lipoxygenase-activating protein (NM_{—}001629.2; SEQ ID NO: 76), IOH11788, signal peptide peptidase 3 (NM_{—}025781.1; SEQ ID NO: 77); IOH13675, leptin receptor (NM_{—}017526.2; SEQ ID NO: 78); IOH7518, microsomal glutathione S-transferase 3 (NM_{—}004528.2; SEQ ID NO: 79); IOH26587, dysbindin (dystrobrevin binding protein 1; SEQ ID NO: 80) (NM_{—}033542.2); IOH57177, PRA1 domain family, member 2 (NM_{—}007213.1; SEQ ID NO: 81); and IOH54702, phosphatidic acid phosphatase type 2 domain containing 1B (NM_{—}032483.2; SEQ ID NO: 82). Following incubation of the protein synthesis reactions, soluble and total reaction aliquots were compared by gel electrophoresis and autoradiography. The amount of synthesized protein was determined by TCA precipitable counts of ³⁵S methionine labeled proteins and calculating an estimated yield from equations using specific activity of isotope/pmoles cold methionine and protein size, and the relative amounts of soluble protein synthesized was determined by determining the TCA precipitable counts of soluble fractions.

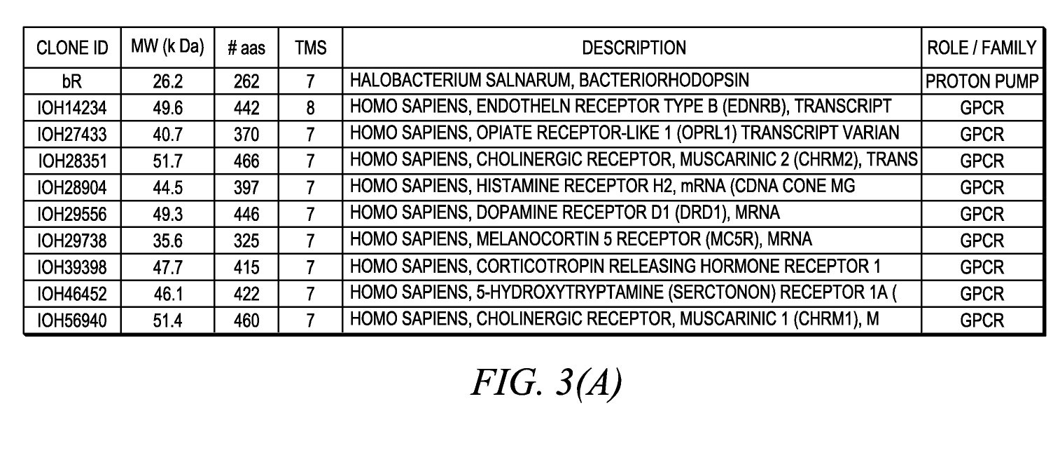

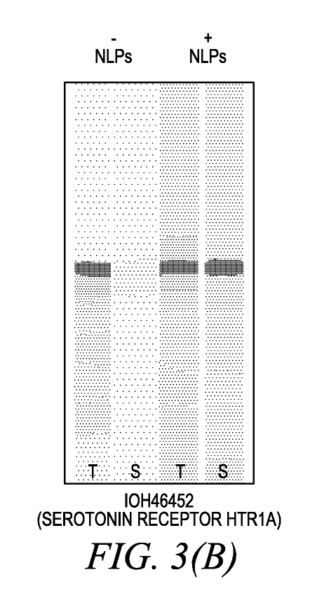

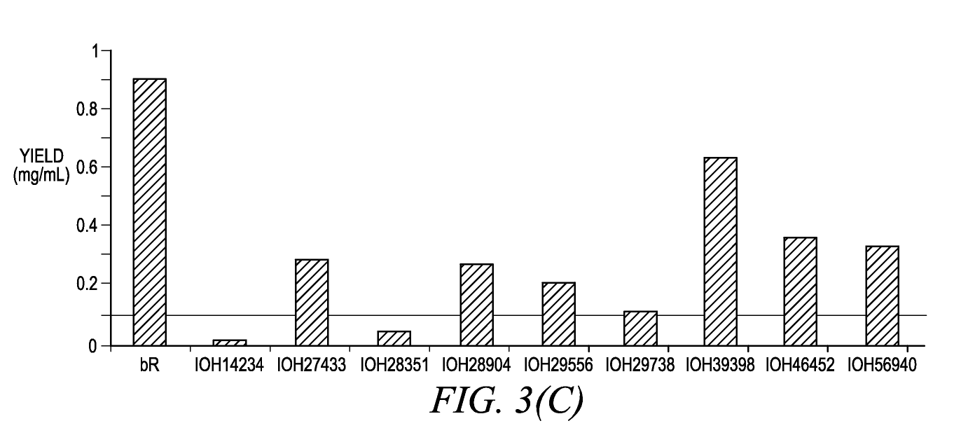

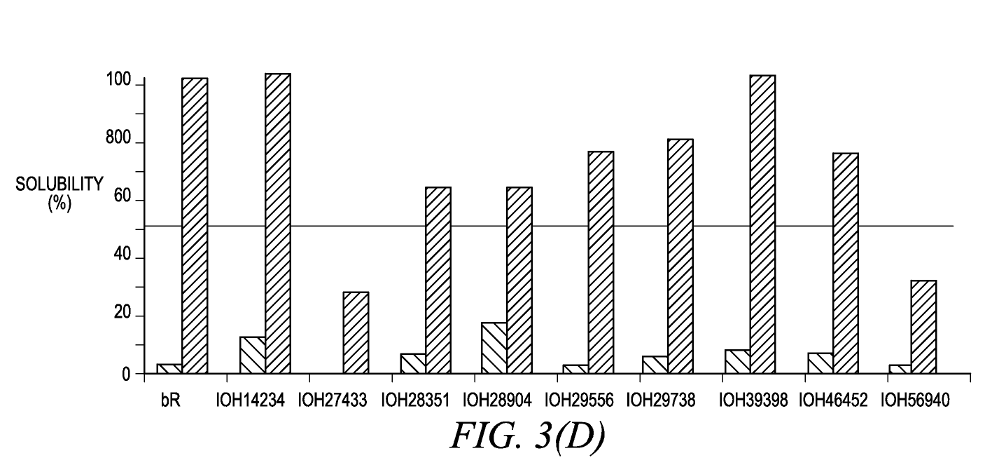

FIG. 3A is a table listing the proteins expressed in these experiments. FIG. 3B shows an autoradiographed gel showing electrophoresed samples of soluble (S) and total (T) protein synthesized in the absence (−) and presence (+) of PPPs for one GPCR protein (serotonin receptor HTR1; IOH46452). FIG. 3C show the yields of several GPCR proteins synthesized in vitro in the presence of PPPs, and FIG. 3D shows that solubility was enhanced by the addition of PPPs to in vitro synthesis reactions. The data demonstrates that solubility was greatly enhanced for the majority of proteins by the inclusion of PPPs in the in vitro synthesis reactions, where the percent solubility was calculated as the amount of synthesized protein present in the soluble fraction divided by the total amount of synthesized protein.

### EXAMPLE 4

### In Vitro Synthesis of Proteins with PPPs in Eukaryotic Extracts

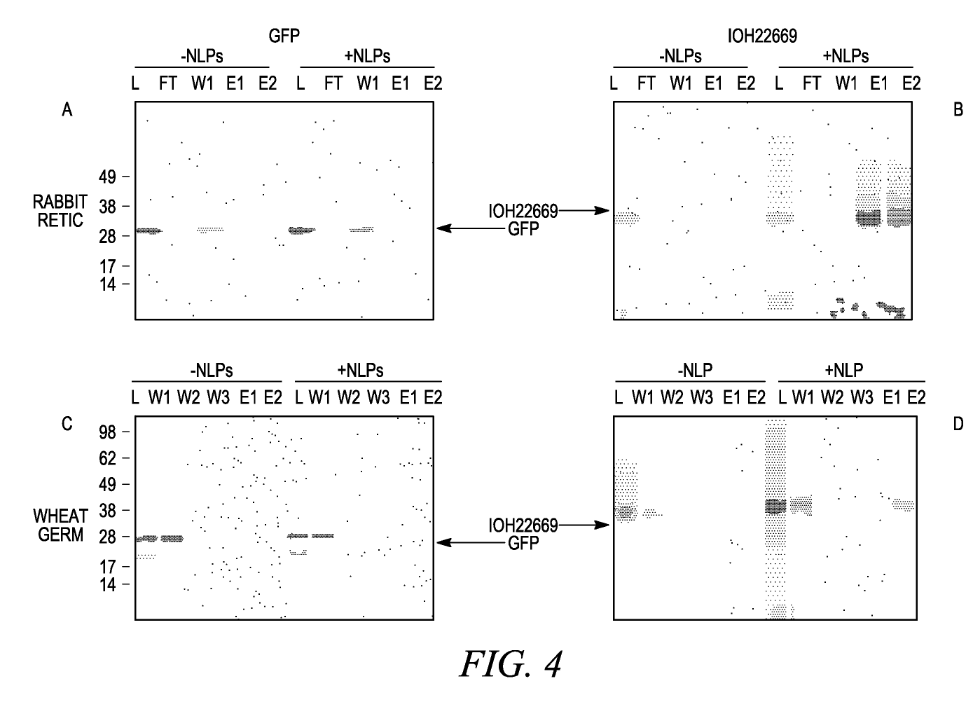

In vitro synthesis of proteins in the presence of PPPs was also tested in rabbit reticulocyte lysate and wheat germ protein synthesis systems. In separate experiments, DNA vectors encoding Green fluorescent protein (GFP), a soluble protein, and a membrane proteins, the human adrenomedullin receptor protein were added to either rabbit reticulocyte lysate in vitro protein synthesis extract (Promega) or a wheat germ in vitro protein synthesis extract and IVPS reactions were performed using radiolabeled ³⁵S methionine according to the manufacturer's instructions, except that PPPs were added to some reaction. The PPPs included the MSP1 membrane scaffold protein which included a his tag. After incubation of the protein synthesis reactions, PPPs were isolated on Ni-NTA resin using the his tag of the scaffold proteins. Aliquots of the reaction products prior to loading on the purification column as well as wash and elution fractions were electrophoresed using SDS PAGE and autoradiography was performed to visualize labeled in vitro synthesized protein. FIG. 4 shows that while GFP, a soluble protein, was synthesized in both the rabbit reticulocyte and wheat germ in vitro synthesis systems, the synthesized GFP did not bind to the affinity column that bound his tagged MSP1. In contrast, the adrenomedullin receptor (membrane protein) was purified on the Ni-NTA column for his tag purification, indicating that the receptor was associated with the scaffold protein present in the added PPPs.

### EXAMPLE 5

### Labeling of PPP with Lipophilic Dyes

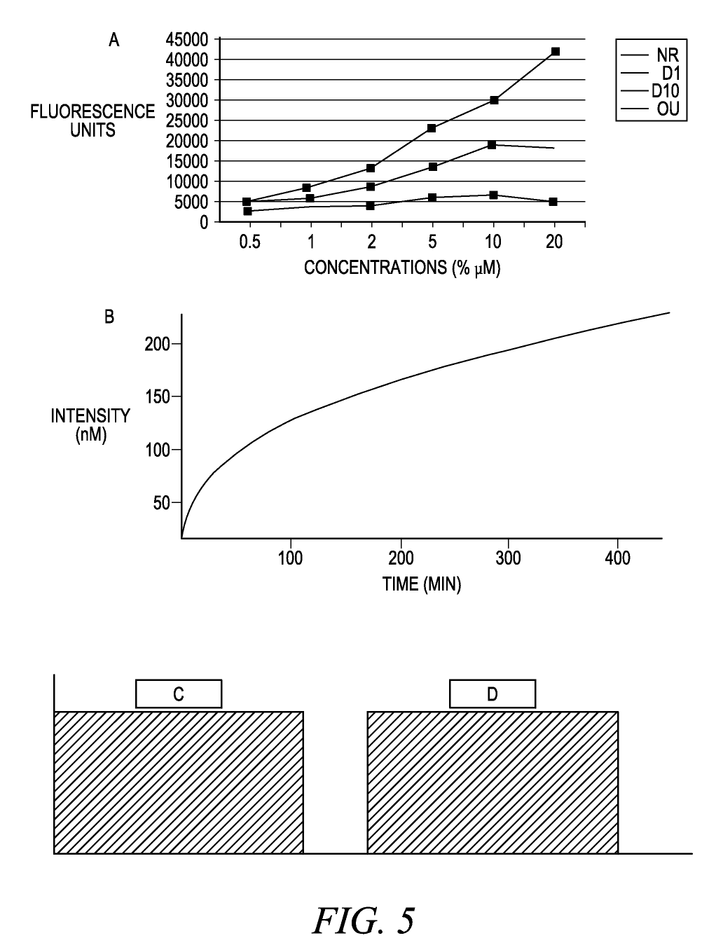

PPPs were diluted to 0.5 micromolar in PBS. The lipophilic dyes DiR, DiI, DiD, DiA were dissolved in DMF to 3 mM. The dyes were then mixed with PPPs at final concentration of 1-10 micromolar and the intensity was monitored using Cary fluorescence spectrophotometer until the maximum intensity was reached. Since the lipophilic dyes only emit fluorescence when they were inserted into lipids, the florescence detected are from the labeled PPPs. The kinetics of DiD insertion into nanodisk is shown in FIG. 5.

### EXAMPLE 6

### FRET Assay of EmrE in PPPs with Lipid Label

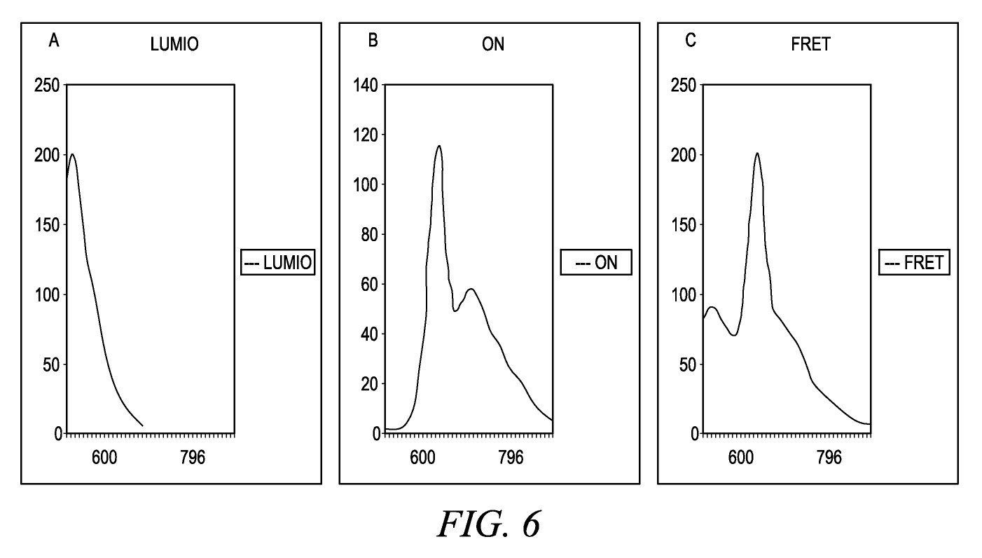

The bacterial EmrE protein synthesized with a LUMIO™ tag was in vitro translated using Invitrogen EXPRESSWAY™ in vitro protein synthesis system in the presence of Nanodisc™ PPPs. The nanodisc with inserted EmrE protein at concentration of 0.5 microMolar was then mixed with DiI (final concentration of 1 miroMolar) for 8 hours. The mixture was tested using Cary Fluorescence Spectrophotometer with Excitation 500 nm/Emission 510-710 nm to confirm the insertion of DiI (DiI is not fluorescent unless in a lipid environment) This confirmed labeling of nanodisc PPPs. Ten microliters of Lumio Green detection reagent (Invitrogen FlAsH Lumio™ green detection kit, cat# LC6090) was then incubated with labeled nanodisc PPPs at room temperature for 10 minutes. FRET was then measured with Cary Fluorescence Spectrophotometer with Excitation 500 nm/Emission 510-710 nm. As controls, the nanodisc-EmrE without DiI labeling and DiI labeled nanodisc-EmrE without adding Lumio Green detection reagent were used. While two controls were confirmed with the emission of fluorescence light peaked at wavelength 535 nm and 580 nm respectively, the DiI labeled nanodisc-EmrE shows enhanced emission at wavelength of 580 nm with excitation of 500 nm, indicating the FRET signal was generated between donor Lumio-FlAsH complex and lipophilic dye DiI (FIG. 6).

### EXAMPLE 7

### EmrE PPP-Ligand Complexes

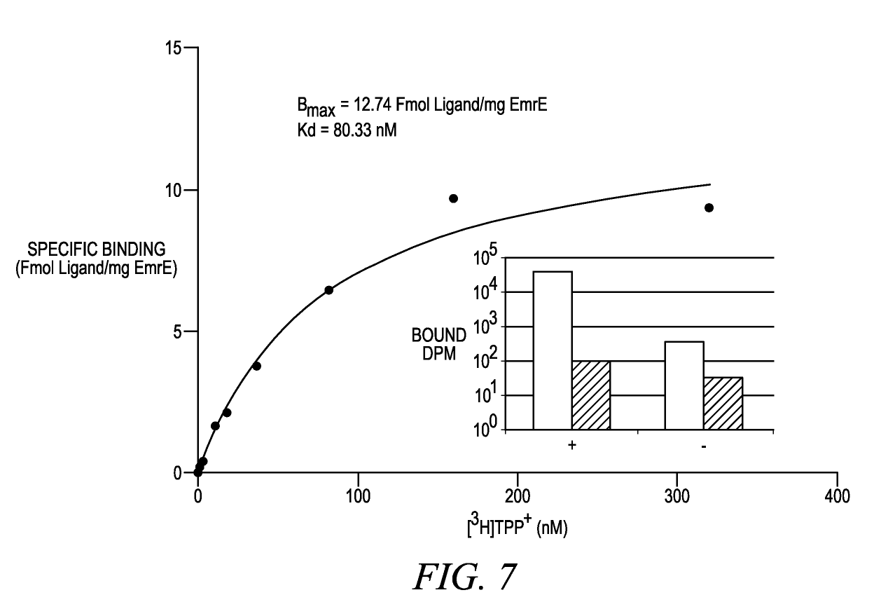

FIG. 7 shows the synthesis of EmrE and bR synthesized in a cell-free protein expression reaction. Equal volumes were incubated with isotope-labeled [³H]tetraphenylphosphonium ([³H]TPP+) in the presence or absence of cold TPP. Excess of TPP was removed by a microspin column containing Sephadex G-50 fine. Remaining radioactive counts were detected by scintillation. As seen in the figure, EmrE expressed in this system is able to bind its [³H]TPP+ ligand.

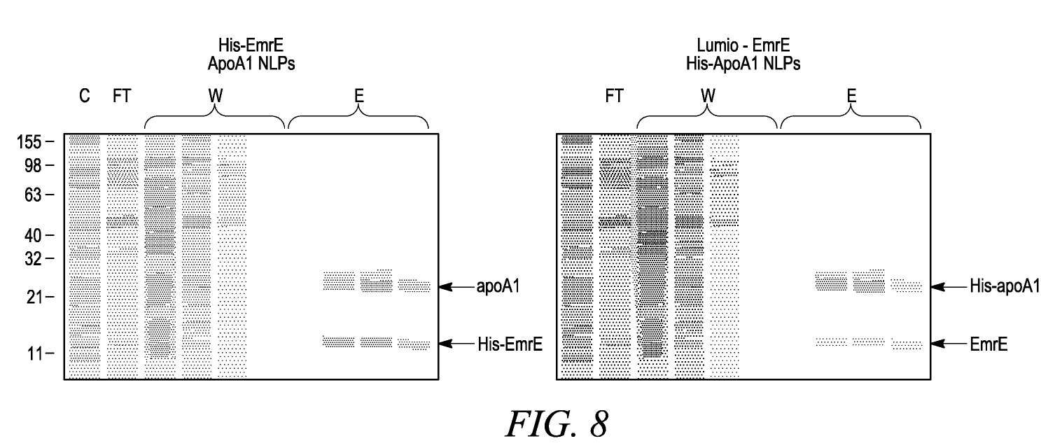

FIG. 8 illustrates the results of a [³H]TPP+ binding analyses. In these experiments, EmrE activity was assayed using a tetraphenylphosphonium (TPP+)-binding assay. Briefly, EmrE was expressed in vitro and immobilized on Ni²⁺-nitrilotriacetic acid (Ni-NTA agarose) beads (Invitrogen, Carlsbad, Calif.). The beads were then washed with binding buffer containing 150 mM NaCl, 10 mM imidazole, 15 mM Tris Cl, pH 7.5, and the protein content was estimated by gel densitometry. One tenth of a microgram of EmrE was added to the binding buffer containing 0.125-320 nM [³H]TPP+ (28 Ci/mmol; GE Healthcare), and incubated for 1 h at room temperature. Nonspecific binding was determined by competition with 20 μM cold TPP+ (Sigma-Aldrich, St. Louis, Mo.). Data points were fitted to a saturation binding curve by nonlinear regression using Prism (GraphPad Software, San Diego, Calif.). For each data point, unspecific binding was determined by subtracting [³H]TPP+ bound in the presence of 20 μM non-radioactive competitor. The DNA source for EmrE was pEXP5-NT-EmrE. In the inset, [³H]TPP+ binding was performed in the absence (empty bars) or presence (filled bars) of non-radioactive TPP+. Binding reactions were carried out with EmrE synthesized in the presence (+) or absence (−) of PPPs. As shown in the figure, binding was higher when EmrE was expressed in the presence of PPPs.

While the present invention has been described in terms of the preferred embodiments, it is understood that variations and modifications will occur to those skilled in the art. Therefore, it is intended that the appended claims cover all such equivalent variations that come within the scope of the invention as claimed.

What is claimed is:

1. An isolated phosphophospholipid-protein particle comprising a scaffold protein and a dye.

2. The isolated phosphophospholipid-protein particle of claim 1, wherein the scaffold protein is a recombinant scaffold protein.

3. The isolated phosphophospholipid-protein particle of claim 1 wherein the dye is selected from the group consisting of a fluorophore, an amphilic dye, a nonpolar dye, and a lipid-partitioning fluorescent molecule.

4. The isolated phosphophospholipid-protein particle of claim 3, wherein the dye is selected from the group consisting of DiI; DiO; DiD; DiR; an analog of DiI, DiO, DiD, or DiR; an amphiphilic derivative of rhodamine; an amphiphilic derivative of fluorescein; an amphiphilic derivative of coumarin; octadecyl rhodamine B; 5-dodecanoyl-aminofluorescein; 5-hexadecanoyl-fluorescein; 5-octadecanolyl-aminofluorescein; 4-heptadecyl-7-hydroxycoumarin; diphenylhexatriene (DPH); trimethylammonium DPH; trimethylammonium phosphate DPH; DPH propionic acid; a nonpolar BODIPY fluorophore; a nonpolar pyrene; Nile red; bimane azide; prodan; laurdan; a dapoxyl derivative; anilinonaphthalenesulfonate (ANS); bis ANS; DCVJ; and, 4-amino-4′-benzamidostilbene-2,2′-disulfonic acid.

5. The isolated phosphophospholipid-protein particle of claim 1, further comprising a membrane protein of interest.

6. The isolated phosphophospholipid-protein particle of claim 5, further comprising a fluorescent protein or fragment thereof.

7. The isolated phospholipid-protein particle of claim 6, wherein the fluorescent protein is selected from the group consisting of GFP, EGFP, BFP, CFP, RFP, YFP, and a protein with at least 80% sequence identity to a native GFP, EGFP, BFP, CFP, RFP, or YFP.

8. A composition comprising an isolated phosphophospholipid-protein particle comprising a scaffold protein, a dye, and a cell extract for performing translation of a nucleic acid template.

9. The composition of claim 8, wherein the scaffold protein is a recombinant scaffold protein.

10. The composition of claim 8, wherein the dye is selected from the group consisting of a fluorophore, an amphilic dye, a nonpolar dye, and a lipid-partitioning fluorescent molecule.

11. The isolated composition of claim 8, wherein the dye is selected from the group consisting of DiI; DiO; DiD; DiR; an analog of DiI, DiO, DiD, or DiR; an amphiphilic derivative of rhodamine; an amphiphilic derivative of fluorescein; an amphiphilic derivative of coumarin; octadecyl rhodamine B; 5-dodecanoyl-aminofluorescein; 5-hexadecanoyl-fluorescein; 5-octadecanolyl-aminofluorescein; 4-heptadecyl-7-hydroxycoumarin; diphenylhexatriene (DPH); trimethylammonium DPH; trimethylammonium phosphate DPH; DPH propionic acid; a nonpolar BODIPY fluorophore; a nonpolar pyrene; Nile red; bimane azide; prodan; laurdan; a dapoxyl derivative; anilinonaphthalenesulfonate (ANS); bis ANS; DCVJ; and, 4-amino-4′-benzamidostilbene-2,2′-disulfonic acid.

12. The composition of claim 8, further comprising a membrane protein of interest.

13. The composition of claim 12, wherein the membrane protein of interest is selected from the group consisting of EmrE (SEQ ID NO: 43), bacteriorhodopsin (SEQ ID NO: 44), a polypeptide expressible from the Invitrogen Ultimate™ ORF clone collection, a G protein-coupled receptor (GPCR), G protein-coupled receptor family C group 5 member C (SEQ ID NO: 45), G protein-coupled receptor 157 (SEQ ID NO: 46), serotonin receptor HTR1 (SEQ ID NO: 47), endothelin receptor type B (SEQ ID NO: 48), opiate receptor-like 1 (SEQ ID NO: 49), cholinergic receptor muscarinic 2 (SEQ ID NO: 50), histamine receptor H2 (SEQ ID NO: 51), dopamine receptor D1 (SEQ ID NO: 52), melanocortin 5 receptor (SEQ ID NO: 53), corticotropin releasing hormone receptor 1 (SEQ ID NO: 54), 5-hydroxytryptamine (serotonin) receptor 1A (SEQ ID NO: 55), cholinergic receptor muscarinic 1 (SEQ ID NO: 56), CD24 (SEQ ID NO: 57), glycophorin E (SEQ ID NO: 58), glycophorin B (SEQ ID NO: 59), chemokine-like factor (SEQ ID NO: 60), glycophorin A (SEQ ID NO: 61), murine microsomal glutathione S-transferase 1 (SEQ ID NO: 62), phosphatidylinositol glycan anchor biosynthesis class P (SEQ ID NO: 63), epiregulin (SEQ ID NO: 64), epiregulin (SEQ ID NO: 65), CD99 (SEQ ID NO: 66), murine Mpv17 transgene (SEQ ID NO: 67), MpV17 mitochondrial inner membrane protein (SEQ ID NO: 68), translocase of inner mitochondrial membrane 22 homolog (SEQ ID NO: 69), ninjurin 2 (SEQ ID NO: 70), signal peptide peptidase-like 2B (SEQ ID NO: 71), CKLF-like MARVEL transmembrane domain containing 1 (SEQ ID NO: 72), golgi transport 1 homolog B (SEQ ID NO: 73), leukotriene C4 synthase (SEQ ID NO: 74), angiotensin II receptor-associated protein (SEQ ID NO: 75), arachidonate 5-lipoxygenase-activating protein (SEQ ID NO: 76), signal peptide peptidase 3 (SEQ ID NO: 77), leptin receptor (SEQ ID NO: 78), microsomal glutathione S-transferase 3 (SEQ ID NO: 79), dystrobrevin binding protein 1 (SEQ ID NO: 80), PRA1 domain family member 2 (SEQ ID NO: 81), phosphatidic acid phosphatase type 2 domain containing 1B (SEQ ID NO: 82), and human adrenomedullin receptor protein (SEQ ID NO: 83).

14. The composition of claim 12, further comprising a fluorescent protein or fragment thereof.

15. The composition of claim 14, wherein the fluorescent protein is selected from the group consisting of GFP, EGFP, BFP, CFP, RFP, or YFP, and a fluorescent protein with at least 80% sequence identity to a native GFP, EGFP, BFP, CFP, RFP, or YFP.

16. The composition of claim 8, further comprising a nucleic acid template encoding a membrane protein of interest.

17. The composition of claim 16 wherein the membrane protein of interest is selected from the group consisting of EmrE (SEQ ID NO: 43), bacteriorhodopsin (SEQ ID NO: 44), a polypeptide expressible from the Invitrogen Ultimate™ ORF clone collection, a G protein-coupled receptor (GPCR), G protein-coupled receptor family C group 5 member C (SEQ ID NO: 45), G protein-coupled receptor 157 (SEQ ID NO: 46), serotonin receptor HTR1 (SEQ ID NO: 47), endothelin receptor type B (SEQ ID NO: 48), opiate receptor-like 1 (SEQ ID NO: 49), cholinergic receptor muscarinic 2 (SEQ ID NO: 50), histamine receptor H2 (SEQ ID NO: 51), dopamine receptor D1 (SEQ ID NO: 52), melanocortin 5 receptor (SEQ ID NO: 53), corticotropin releasing hormone receptor 1 (SEQ ID NO: 54), 5-hydroxytryptamine (serotonin) receptor 1A (SEQ ID NO: 55), cholinergic receptor muscarinic 1 (SEQ ID NO: 56), CD24 (SEQ ID NO: 57), glycophorin E (SEQ ID NO: 58), glycophorin B (SEQ ID NO: 59), chemokine-like factor (SEQ ID NO: 60), glycophorin A (SEQ ID NO: 61), murine microsomal glutathione S-transferase 1 (SEQ ID NO: 62), phosphatidylinositol glycan anchor biosynthesis class P (SEQ ID NO: 63), epiregulin (SEQ ID NO: 64), epiregulin (SEQ ID NO: 65), CD99 (SEQ ID NO: 66), murine Mpv17 transgene (SEQ ID NO: 67), MpV17 mitochondrial inner membrane protein (SEQ ID NO: 68), translocase of inner mitochondrial membrane 22 homolog (SEQ ID NO: 69), ninjurin 2 (SEQ ID NO: 70), signal peptide peptidase-like 2B (SEQ ID NO: 71), CKLF-like MARVEL transmembrane domain containing 1 (SEQ ID NO: 72), golgi transport 1 homolog B (SEQ ID NO: 73), leukotriene C4 synthase (SEQ ID NO: 74), angiotensin II receptor-associated protein (SEQ ID NO: 75), arachidonate 5-lipoxygenase-activating protein (SEQ ID NO: 76), signal peptide peptidase 3 (SEQ ID NO: 77), leptin receptor (SEQ ID NO: 78), microsomal glutathione S-transferase 3 (SEQ ID NO: 79), dystrobrevin binding protein 1 (SEQ ID NO: 80), PRA1 domain family member 2 (SEQ ID NO: 81), phosphatidic acid phosphatase type 2 domain containing 1B (SEQ ID NO: 82), and human adrenomedullin receptor protein (SEQ ID NO: 83).

18. A kit comprising a cell extract, a ligand, and an isolated phospholipid-protein particle comprising a scaffold protein and a phospholipid.

19. The kit of claim 18 wherein the ligand is a ligand of a membrane protein is selected from the group consisting of EmrE (SEQ ID NO: 43), bacteriorhodopsin (SEQ ID NO: 44), a polypeptide expressible from the Invitrogen Ultimate™ ORF clone collection, a G protein-coupled receptor (GPCR), G protein-coupled receptor family C group 5 member C (SEQ ID NO: 45), G protein-coupled receptor 157 (SEQ ID NO: 46), serotonin receptor HTR1 (SEQ ID NO: 47), endothelin receptor type B (SEQ ID NO: 48), opiate receptor-like 1 (SEQ ID NO: 49), cholinergic receptor muscarinic 2 (SEQ ID NO: 50), histamine receptor H2 (SEQ ID NO: 51), dopamine receptor D1 (SEQ ID NO: 52), melanocortin 5 receptor (SEQ ID NO: 53), corticotropin releasing hormone receptor 1 (SEQ ID NO: 54), 5-hydroxytryptamine (serotonin) receptor 1A (SEQ ID NO: 55), cholinergic receptor muscarinic 1 (SEQ ID NO: 56), CD24 (SEQ ID NO: 57), glycophorin E (SEQ ID NO: 58), glycophorin B (SEQ ID NO: 59), chemokine-like factor (SEQ ID NO: 60), glycophorin A (SEQ ID NO: 61), murine microsomal glutathione S-transferase 1 (SEQ ID NO: 62), phosphatidylinositol glycan anchor biosynthesis class P (SEQ ID NO: 63), epiregulin (SEQ ID NO: 64), epiregulin (SEQ ID NO: 65), CD99 (SEQ ID NO: 66), murine Mpv17 transgene (SEQ ID NO: 67), MpV17 mitochondrial inner membrane protein (SEQ ID NO: 68), translocase of inner mitochondrial membrane 22 homolog (SEQ ID NO: 69), ninjurin 2 (SEQ ID NO: 70), signal peptide peptidase-like 2B (SEQ ID NO: 71), CKLF-like MARVEL transmembrane domain containing 1 (SEQ ID NO: 72), golgi transport 1 homolog B (SEQ ID NO: 73), leukotriene C4 synthase (SEQ ID NO: 74), angiotensin II receptor-associated protein (SEQ ID NO: 75), arachidonate 5-lipoxygenase-activating protein (SEQ ID NO: 76), signal peptide peptidase 3 (SEQ ID NO: 77), leptin receptor (SEQ ID NO: 78), microsomal glutathione S-transferase 3 (SEQ ID NO: 79), dystrobrevin binding protein 1 (SEQ ID NO: 80), PRA1 domain family member 2 (SEQ ID NO: 81), phosphatidic acid phosphatase type 2 domain containing 1B (SEQ ID NO: 82), and human adrenomedullin receptor protein (SEQ ID NO: 83).

20. The kit of claim 18 wherein the phospholipid is selected from the group consisting of phosphatidyl choline, phosphatidyl ethanolamine, phosphatidyl inositol, dipalmitoyl-phosphatidylcholine, dimyristoyl phosphatidyl choline, 1-palmitoyl-2-oleoyl-phosphatidyl choline, dihexanoyl phosphatidyl choline, dipalmitoyl phosphatidyl ethanolamine, dipalmitoyl phosphatidyl inositol, dimyristoyl phosphatidyl ethanolamine, dimyristoyl phosphatidyl inositol, dihexanoyl phosphatidyl ethanolamine, dihexanoyl phosphatidyl inositol, 1-palmitoyl-2-oleoyl-phosphatidyl ethanolamine, and 1-palmitoyl-2-oleoyl-phosphatidyl inositol.

21. The kit of claim 18 wherein the scaffold protein is selected from the group consisting of an apolipoprotein, apolipoprotein A1 (SEQ ID NO: 1), MSP1 (SEQ ID NO: 20), synuclein alpha (SEQ ID NO:84), synuclein alpha (SEQ ID NO:85), synuclein beta (SEQ ID NO:86), synuclein beta (SEQ ID NO:87), synuclein gamma (SEQ ID NO:88), apomyoglobin, a peptabiol, melitin, almethicin, gramicidin, and variants thereof.

22. The kit of claim 18 further comprising a dye selected from the group consisting of a fluorophore, an amphilic dye, a nonpolar dye, and a lipid-partitioning fluorescent molecule.

23. The kit of claim 22, wherein the dye is selected from the group consisting of DiI; DiO; DiD; DiR; an analog of DiI, DiO, DiD, or DiR; an amphiphilic derivative of rhodamine; an amphiphilic derivative of fluorescein; an amphiphilic derivative of coumarin; octadecyl rhodamine B; 5-dodecanoyl-aminofluorescein; 5-hexadecanoyl-fluorescein; 5-octadecanolyl-aminofluorescein; 4-heptadecyl-7-hydroxycoumarin; diphenylhexatriene (DPH); trimethylammonium DPH; trimethylammonium phosphate DPH; DPH propionic acid; a nonpolar BODIPY fluorophore; a nonpolar pyrene; Nile red; bimane azide; prodan; laurdan; a dapoxyl derivative; anilinonaphthalenesulfonate (ANS); bis ANS; DCVJ; and, 4-amino-4′-benzamidostilbene-2,2′-disulfonic acid.

24. The kit of claim 18, further comprising a membrane protein of interest or nucleic acid template encoding a membrane protein of interest.

25. The kit of claim 24 wherein the membrane protein of interest is selected from the group consisting of EmrE (SEQ ID NO: 43), bacteriorhodopsin (SEQ ID NO: 44), a polypeptide expressible from the Invitrogen Ultimate™ ORF clone collection, a G protein-coupled receptor (GPCR), G protein-coupled receptor family C group 5 member C (SEQ ID NO: 45), G protein-coupled receptor 157 (SEQ ID NO: 46), serotonin receptor HTR1 (SEQ ID NO: 47), endothelin receptor type B (SEQ ID NO: 48), opiate receptor-like 1 (SEQ ID NO: 49), cholinergic receptor muscarinic 2 (SEQ ID NO: 50), histamine receptor H2 (SEQ ID NO: 51), dopamine receptor D1 (SEQ ID NO: 52), melanocortin 5 receptor (SEQ ID NO: 53), corticotropin releasing hormone receptor 1 (SEQ ID NO: 54), 5-hydroxytryptamine (serotonin) receptor 1A (SEQ ID NO: 55), cholinergic receptor muscarinic 1 (SEQ ID NO: 56), CD24 (SEQ ID NO: 57), glycophorin E (SEQ ID NO: 58), glycophorin B (SEQ ID NO: 59), chemokine-like factor (SEQ ID NO: 60), glycophorin A (SEQ ID NO: 61), murine microsomal glutathione S-transferase 1 (SEQ ID NO: 62), phosphatidylinositol glycan anchor biosynthesis class P (SEQ ID NO: 63), epiregulin (SEQ ID NO: 64), epiregulin (SEQ ID NO: 65), CD99 (SEQ ID NO: 66), murine Mpv17 transgene (SEQ ID NO: 67), MpV17 mitochondrial inner membrane protein (SEQ ID NO: 68), translocase of inner mitochondrial membrane 22 homolog (SEQ ID NO: 69), ninjurin 2 (SEQ ID NO: 70), signal peptide peptidase-like 2B (SEQ ID NO: 71), CKLF-like MARVEL transmembrane domain containing 1 (SEQ ID NO: 72), golgi transport 1 homolog B (SEQ ID NO: 73), leukotriene C4 synthase (SEQ ID NO: 74), angiotensin II receptor-associated protein (SEQ ID NO: 75), arachidonate 5-lipoxygenase-activating protein (SEQ ID NO: 76), signal peptide peptidase 3 (SEQ ID NO: 77), leptin receptor (SEQ ID NO: 78), microsomal glutathione S-transferase 3 (SEQ ID NO: 79), dystrobrevin binding protein 1 (SEQ ID NO: 80), PRA1 domain family member 2 (SEQ ID NO: 81), phosphatidic acid phosphatase type 2 domain containing 1B (SEQ ID NO: 82), and human adrenomedullin receptor protein (SEQ ID NO: 83)

26. The kit of claim 18, further comprising a fluorescent protein of fragment thereof.

27. The kit of claim 18, wherein the fluorescent protein is selected from the group consisting of GFP, EGFP, BFP, CFP, RFP, or YFP, and a fluorescent protein with at least 80% sequence identity to a native GFP, EGFP, BFP, CFP, RFP, or YFP.


SEQUENCE LISTING (89 sequences)

SEQ ID NO: 1 - protein, 267 aa, Homo Sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 250 aa, Homo Sapiens
MAHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 3 - protein, 100 aa, Homo Sapiens
MKLLAATVLLLTICSLEGALVRRQAKEPCVESLVSQYFQTVTDYGKDLMEKVKSPELQAEAKSYFEKSKEQLTPLIKKAGTELVNFLSYFVELGTQPATQ

SEQ ID NO: 4 - protein, 396 aa, Homo Sapiens
MFLKAVVLTLALVAVAGARAEVSADQVATVMWDYFSQLSNNAKEAVEHLQKSELTQQLNALFQDKLGEVNTYAGDLQKKLVPFATELHERLAKDSEKLKEEIGKELEELRARLLPHANEVSQKIGDNLRELQQRLEPYADQLRTQVNTQAEQLRRQLDPLAQRMERVLRENADSLQASLRPHADELKAKIDQNVEELKGRLTPYADEFKVKIDQTVEELRRSLAPYAQDTQEKLNHQLEGLTFQMKKNAEELKARISASAEELRQRLAPLAEDVRGNLKGNTEGLQKSLAELGGHLDQQVEEFRRRVEPYGENFNKALVQQMEQLRQKLGPHAGDVEGHLSFLEKDLRDKVNSFFSTFKEKESQDKTLSLPELEQQQEQQQEQQQEQVQMLAPLES

SEQ ID NO: 5 - protein, 366 aa, Homo Sapiens
MASMAAVLTWALALLSAFSATQARKGFWDYFSQTSGDKGRVEQIHQQKMAREPATLKDSLEQDLNNMNKFLEKLRPLSGSEAPRLPQDPVGMRRQLQEELEEVKARLQPYMAEAHELVGWNLEGLRQQLKPYTMDLMEQVALRVQELQEQLRVVGEDTKAQLLGGVDEAWALLQGLQSRVVHHTGRFKELFHPYAESLVSGIGRHVQELHRSVAPHAPASPARLSRCVQVLSRKLTLKAKALHARIQQNLDQLREELSRAFAGTGTEEGAGPDPQMLSEEVRQRLQAFRQDTYLQIAAFTRAIDQETEEVQQQLAPPPPGHSAFAPEFQQTDSGKVLSKLQARLDDLWEDITHSLHDQGHSHLGDP

SEQ ID NO: 6 - protein, 4563 aa, Homo Sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYNNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPETDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSCDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVVENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDVLEHVKHFVINLIGDFEVAEKINAFRAKVHELIERYEVDQQIQVLMDKLVELTHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMQNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPHFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVAKTTKAEIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFITPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTLAHRDFSAEYEEDGKFEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQKFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFNDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 7 - protein, 728 aa, Homo Sapiens
LNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDVLEHVKHFVINPYWDFEVAEKINAFRAKVHELIERYEVDQHIQVLMDKLVELAHQYKLKETIQKLSNVLQQVKIKDYFEKLVGFID

SEQ ID NO: 8 - protein, 83 aa, Homo Sapiens
MRLFLSLPVLVVVLSIVLEGPAPAQGTPDVSSALDKLKEFGNTLEDKARELISRIKQSELSAKMREWFSETFQKVKEKLKIDS

SEQ ID NO: 9 - protein, 101 aa, Homo Sapiens
MGTRLLPALFLVLLVLGFEVQGTQQPQQDEMPSPTFLTQVKESLSSYWESAKTAAQNLYEKTYLPAVDEKLRDLYSKSTAAMSTYTGIFTDQVLSVLKGEE

SEQ ID NO: 10 - protein, 99 aa, Homo Sapiens
MQPRVLLVVALLALLASARASEAEDASLLSFMQGYMKHATKTAKDALSSVQESQVAQQARGWVTDGFSSLKDYWSTVKDKFSEFWDLDPEVRPASAVAA

SEQ ID NO: 11 - protein, 189 aa, Homo Sapiens
MVMLLLLLSALAGLFGAAEGQAFHLGKCPNPPVQENFDVNKYLGRWYEIEKIPTTFENGRCIQANYSLMENGKIKVLNQELRADGTVNQIEGEATPVNLTEPAKLEVKFSWFMPSAPYWILATDYENYALVYSCTCIIQLFHVDFAWILARNPNLPPETVDSLKNILTSNNIDVKKMTVTDQVNCPKLS

SEQ ID NO: 12 - protein, 98 aa, Homo Sapiens
GEATPVNLTEPAKLEVKFSWFMPSAPYWILATDYENYALVYSCTCIIQLFHVDFAWILARNPNLPPETVDSLKNILTSNNIDVKKMTVTDQVNCPKLS

SEQ ID NO: 13 - protein, 317 aa, Homo Sapiens [sequence omitted]

SEQ ID NO: 14 - protein, 345 aa, Homo Sapiens
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 15 - protein, 4548 aa, Homo Sapiens
MEHKEVVLLLLLFLKSAAPEQSHVVQDCYHGDGQSYRGTYSTTVTGRTCQAWSSMTPHQHNRTTENYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDPVAAPYCYTRDPSVRWEYCNLTQCSDAEGTAVAPPTITPIPSLEAPSEQAPTEQRPGVQECYHGNGQSYQGTYFITVTGRTCQAWSSMTPHSHSRTPAYYPNAGLIKNYCRNPDPVAAPWCYTTDPSVRWEYCNLTRCSDAEWTAFVPPNVILAPSLEAFFEQALTEETPGVQDCYYHYGQSYRGTYSTTVTGRTCQAWSSMTPHQHSRTPENYPNAGLTRNYCRNPDAEIRPWCYTMDPSVRWEYCNLTQCLVTESSVLATLTVVPDPSTEASSEEAPTEQSPGVQDCYHGDGQSYRGSFSTTVTGRTCQSWSSMTPHWHQRTTEYYPNGGLTRNYCRNPDAEISPWCYTMDPNVRWEYCNLTQCPVTESSVLATSTAVSEQAPTEQSPTVQDCYHGDGQSYRGSFSTTVTGRTCQSWSSMTPHWHQRTTEYYPNGGLTRNYCRNPDAEIRPWCYTMDPSVRWEYCNLTQCPVMESTLLTTPTVVPVPSTELPSEEAPTENSTGVQDCYRGDGQSYRGTLSTTITGRTCQSWSSMTPHWHRRIPLYYPNAGLTRNYCRNPDAEIRPWCYTMDPSVRWEYCNLTRCPVTESSVLTTPTVAPVPSTEAPSEQAPPEKSPVVQDCYHGDGRSYRGISSTTVTGRTCQSWSSMIPHWHQRTPENYPNAGLTENYCRNPDSGKQPWCYTTDPCVRWEYCNLTQCSETESGVLETPTVVPVPSMEAHSEAAPTEQTPVVRQCYHGNGQSYRGTFSTTVTGRTCQSWSSMTPHRHQRTPENYPNDGLTMNYCRNPDADTGPWCFTMDPSIRWEYCNLTRCSDTEGTVVAPPTVIQVPSLGPPSEQDCMFGNGKGYRGKKATTVTGTPCQEWAAQEPHRHSTFIPGTNKWAGLEKNYCRNPDGDINGPWCYTMNPRKLFDYCDIPLCASSSFDCGKPQVEPKKCPGSIVGGCVAHPHSWPWQVSLRTRFGKHFCGGTLISPEWVLTAAHCLKKSSRPSSYKVILGAHQEVNLESHVQEIEVSRLFLEPTQADIALLKLSRPAVITDKVMPACLPSPDYMVTARTECYITGWGETQGTFGTGLLKEAQLLVIENEVCNHYKYICAEHLARGTDSCQGDSGGPLVCFEKDKYILQGVTSWGLGCARPNKPGVYARVSRFVTWIEGMMRNN

SEQ ID NO: 16 - protein, 3305 aa, Manduca sexta
MGKSNRLLSVLFVISVLWKAAYGNGKCQIACKGSSSPSFAAGQKYNYGVEGTVSVYLTGADNQETSLKMLGQASVSAISNCELELSVHNMVLSGPDGKKYPCPQGIEKPVRFSYQDGRVGPEICAAEDDSRRSLNIKRAIISLLQAEQKPSVQVDVFGVCPTEVSSSQEGGAVLLHRSRDLSRCAHREQGRNDFVNSIANPDAGIKDLQVLQSMLNVESKVNNGVPEKVSAIEEYLYKPFSVGENGARAKVHTKLTLSGKGGAGGGNAHCTESRSIIFDVPHGTSSASGNLNSVISAVKETARTVANDASSKSAGQFAQLVRIMRTSSKDDLMRIYSQVKAHQLEKRVYLDALLRAGTGESIEASIQILKSKDLSQLEQHLVFLSLGNARHVNNPALKAAAGLLDMPNLPKEVYLGAGALGGAYCREHDCHNVKPEGIVALSNKLGSKLQNCRPKNKPDEDVVVAILKGIRNIRHLEDSLIDKLVHCAVDNNVKARVRAVALEAFHADPCSAKIHKTAMDIMKNRQLDSEIRIKAYLAVIECPCSHSASEIKNLLDSEPVHQVGNFITSSLRHIRSSSNPDKQLAKKHYGQIRTPNKFKVDERKYSFYREMSYKLDALGAGGSVDQTVIYSQTSFLPRSVNFNLTVDLFGQSYNVMELGGRQGNLDRVVEHFLGPKSFLRTEDPQALYDNLVKRFQESKKKVEDSLSRGRRSIKSEIDVFDKNLKAESAPYNNELDLDIYVKLFGTDAVFLSFGDDKGFDFNKMLDQILGGCNSGINKAKHFQQEIRSHLLFMDAELAYPTSVGLPLRLNLIGAATARLDVATNIDIRQIFQSPQNAKADIKFVPSTDFEISGAFIIDADAFSTGIKVITNLHSSTGVHVNAKVLENGRGIDLQIGLPVDKQELIAASSDLVFVTAEKGQKEKQKVIKMEKGENEYSACFDQLSGPLGLTMCYDMVLPFPIVNRNDKLDSIAKAMGKWPLSGSAKFKLFLEKNDLRGYHIKAVVKEDKDAGRRSFELLLDTEGAKTRRSQLTGEAVYNENEVGVKLGLEAVGKVIYGHIWAHKKPNELVASVKGKLDDIEYSGKLGFSVQGNEHRAVYKPIFEYSLPDGSSPGSKKYEVKIDGQVIRECDGRVTKYTFDGVHVNLQNAEKPLEICGSVSTVAQPREVEFDVEVKHYASLKGSWKGSDVVLAFNNQLNPKINFDLKGKFENTDSMHNELDIHYGPNRGDNNARITFSQILKYHVENSKNFNVITKNNLEIRAVPFKLVANADVDPKKIDIDIEGQLQDKSAGFNLDARTHIKKEGDYSIKVKANLNNANLEAFSRRDIVNAEKSNVENYIDMKGVGRYELSGFVLHKTKPNDVNVGFIGHLKINGGGKNEDFKINIGHIETPAVFSSHATISGSRGDIIDYLLKIMRTANPNGNFKLVIKDSIAANGQYKVTDADGKGNGLIIIDFKKINRKIKGDVRFTAKEPVFNADIDLFLNFEKDNSDKVHFSTYNKKTDKVMDTKNKLEYAGKRTEVNIHQDGILAVTGKAHTVAELVLPTERCLSLKIDHDGAFKDGLYNGHMDMTISDAPKRGSGASTISYKGKVSNSNLDQEIIDYEGQINFKLKDGKNLQSTFSLKNNPDGDKFKYEFKSDVNGNLIPKPANLVATGTYSNSENEIDETYRLKGSYGSDIGFELAGVGTIKFLDAGDKKYLDDYTLTVRLPFEKAHDIKWVSTVLFLQPQGQEMTEYTLVESVQINADVYKIDANGKVGPKNGYGAVKVLVPHVEPFVLDYNYKSSHEGEKNNNYVELKTKYGKGKSASMVVDSSYAPHYSTLKVKANTPNNDKFKKLDVTVHSKNPSPDAYSNSVVVDADGRVYKIDSSIVLSKAHPVLDIQYHSPSSDKIRRLYLQGSSLSSTQGKLEVKVDNINDICLDAVSEANVQKDNVAFKVVANAKELGWKNYGIDISSKDSGSGKRLEFHATNDNKNVLSGSTSFISKQEGQKTIIEGSGSVKVKEEQKSANFKYIRTVFTDSNEKGVETFFNVALGERSYVAESRVTNYEYKNSYVYCEEKKQCAHAEIQSKIDMSTPGMIVNVINAGLDLRKLGVAPELGLQMRDEVSDRRPPRFTLDLHINKEDRKYHLHAYNTPENGHYASGVTVRLPSRVMALEYTLTHPTSQDLPFPIKGEACLDLDKNRPGHKTSARFLVDYSNSGSEDKAVAEIGFFHPKIEKEAVIRLNAFMKRPENGCFKIESSASLCHSALGTDRVAKVMFETTPNSVKFLADTPFVKAIDVEGSFNVNQQQRTQQCLFRICLLEGKPVQMSALVKDYQYYEFTTEESNRKLSYVGHLIPEKRVDISTDIILSGDKKNIAHGALFLQDNLVKSDYGLSKENFNYFLNALKKDLDTLEDRIKNVGEKASKDVEAVTQRAAPYFKKVEDNFRAEWNRFYQEIADDKVFKEISHVFNEIVQYIAKFIDEILQGTKRSWTPSCRPTLSHPRNREMYKKQIEPQVKQLYDTLGALMKEYLDGVIDVVAHFAAIVTDFFEKHKAELQELTNVFTEIFKDLTRLVVAQLKELPPKIAQIYNDIVSQITNMPFVVVLQEKWKEFNFAERAVQLVSQAYEAFSKILPTDELKEFAKALNAYLLKKIKEEKMEESKELPRAVREAGQRVLLITSIPALAVRRPRLRRWTWHHLKLAVGAGASAPSLGAASWSALRQLAAGDGPPALAPRGLPTAQLDPLDEVPNKLRAVVVNGQHIFTFDGRHLTFPGTCRYVLIHDHVDRNFTVLMQLANGQPKALVLEDKSGTIIELKDNGQVILNCQSHGFPVVEQDVFAFRQTSGRIGLCSKYGLMAFCTSKFEVCYFEVNGFYLGKLPGLLGDGNNEPYDDFRMPNGKICSSESEFGNSYRLSRSCPAANAPAHDHHQMHAPLPKPCERVFSGTSPLRPLSLMLDIAPFRQACIHAVTGADADKDLQQACDLARGYRRSRSRGCCPPRCPTPACAARTATGPGSWATPTSTNCPTDSLISSSPLRPLRTTPAHYKNMVVPLVSQLVDMLKGKHCTDIKVFLVGHTSKHPYPILYDTDLKLKNAKVSFDDKSRYDRIPFVKTGHEKFDSYSKTVVDFLNYIKIELGITNIEASQGQIFDLPLRPGAVKHVIFVTGGPTISQFFLLETVRALRNKVIIDEMAMSASLVTSTPGLKIGGGKNAAQIVGYEKHGVLLLGEKKQSKDSEAVRATLEVEDDPFSDAVEFANGVVFSASNYAALPAGQQKQFIQTAAHNIIQRMWREQIVQQCTCVFVDPFRVRSVCFNKARTEVARRRK

SEQ ID NO: 17 - protein, 386 aa, Aedes aegypti
QQTFKNGVLESVKLGEEYKYVPFAKLNSGAQAKVTTKLTYTGTKAGAAPALTAGAPRSVIFENPQTDSQGNLETIKQELKTVVDSYSQNNVGKLTASHFTELVHLMRFSKKDDLLSLYQQVKAGNAHKNKLLARKVYFDALFRAGTGASVEALANLYKNKEVSDAKEQKLLFVSLNLVTSMTKPALKAAKLLLDGNPSREAYLSVGSLVNKYCQKFGCESADVKEISDKFSAKLGKCQPTTRQEEDTIVAVLKGIKNSNTLVAQLLDKVVGCASDKSSARVRVAAFQAYPAASCNKKIVNSALNFLKNVNEDSEIRIQAYLSPVECPSAAVANEIKALLDNEKVYQVGSFLTTHLASLRASADPTRDAARQHFANIRTTNQFPFDF

SEQ ID NO: 18 - protein, 189 aa, Manduca sexta
MAAKFVVVLAACVALSHSAMVRRDAPAGGNAFEEMEKHAKEFQKTFSEQFNSLVNSKNTQDFNKALKDGSDSVLQQLSAFSSSLQGAISDANGKAKEALEQARQNVEKTAEELRKAHPDVEKEANAFKDKLQAAVQTTVQESQKLAKEVASNMEETNKKLAPKIKQAYDDFVKHAEEVQKKLHEAATKQ

SEQ ID NO: 19 - protein, 212 aa, Artificial Sequence
  engineered apolipoprotein-histidine tagged MSP1
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 20 - protein, 201 aa, Artificial Sequence
  engineered apolipoprotein- MSP1
MAKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 21 - protein, 414 aa, Artificial Sequence
  engineered apolipoprotein- MSP2 -his tagged
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 22 - protein, 422 aa, Artificial Sequence
  engineered apolipoprotein- MSP2 (his tagged, long linker)
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTGGGSGGGTLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 23 - protein, 168 aa, Artificial Sequence
  engineered apolipoprotein- MSP1D5D6
MGHHHHHHIEGRLKLLDNWDSVTSTFSKLREQLGPVTQEFWDNLEKETEGLRQEMSKDLEEVKAKVQPYLDDFQKKWQEEMELYRQKVEPYSDELRQRLAARLEALKENGGARLAEYHAKATEHLSTLSEKAKPALEDLRQGLLPVLESFKVSFLSALEEYTKKLNTQ

SEQ ID NO: 24 - protein, 168 aa, Artificial Sequence
  engineered apolipoprotein-MSP1D6D7
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSARLAEYHAKATEHLSTLSEKAKPALEDLRQGLLPVLESFKVSFLSALEEYTKKLNTQ

SEQ ID NO: 25 - protein, 201 aa, Artificial Sequence
  engineered apolipoprotein-MAP1T4
MGHHHHHHHDYDIPTTENLYFQGSVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 26 - protein, 190 aa, Artificial Sequence
  engineered apolipoprotein-MSP1T5
MGHHHHHHHDYDIPTTENLYFQGKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 27 - protein, 179 aa, Artificial Sequence
  engineered apolipoprotein-MSP1T6
MGHHHHHHHDYDIPTTENLYFQGKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 28 - protein, 199 aa, Artificial Sequence
  engineered apolipoprotein-MSP1N1
MGHHHHHHHDYDIPTTENLYFQGSVTQEFWDNLEKETEGLRQEMSKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLL

SEQ ID NO: 29 - protein, 289 aa, Artificial Sequence
  engineered apolipoprotein-MSP1E3TEV
MGHHHHHHHDYDIPTTENLYFQGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 30 - protein, 278 aa, Artificial Sequence
  engineered apolipoprotein-MSP1E3D1
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 31 - protein, 423 aa, Artificial Sequence
  engineered apolipoprotein-HisTEV-MSP2
MGHHHHHHHDYDIPTTENLYFQGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEYTKKLNTQGTLKLLDNWDSVTSTFSKLREQLGPVTQEFWDNLEKETEGLRQEMKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 32 - protein, 401 aa, Artificial Sequence
  engineered apolipoprotein-MSP2N1
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 33 - protein, 392 aa, Artificial Sequence
  engineered apolipoprotein-MSP2N2
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 34 - protein, 397 aa, Artificial Sequence
  engineered apolipoprotein-MSP2N3
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 35 - protein, 383 aa, Artificial Sequence
  engineered apolipoprotein-MSP2N4
MGHHHHHHHDYDIPTTENLYFQGSVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQNPGTPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 36 - protein, 379 aa, Artificial Sequence
  engineered apolipoprotein-MSP2N5
MGHHHHHHHDYDIPTTENLYFQGSVTQEFWDNLEKETEGLRQEMSKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLNPGTKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVTQEFWDNLEKETEGLRQEMS

SEQ ID NO: 37 - protein, 381 aa, Artificial Sequence
  engineered apolipoprotein-MSP2N6
MGHHHHHHHDYDIPTTENLYFQGSVTQEFWDNLEKETEGLRQEMSKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLSNPGTQKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVTQEFWDNLEKETEGLRQEMS

SEQ ID NO: 38 - protein, 1094 aa, Artificial Sequence
  engineered apolipoprotein-MSP2CPR
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQSTMGDSHEDTSATMPEAVAEEVSLFSTTDMVLFSLIVGVLTYWFIFRKKKEEIPEFSKIQTTAPPVKESSFVEKMKKTGRNIIVFYGSQTGTAEEFANRLSKDAHRYGMRGMSADPEEYDLADLSSLPEIDKSLVVFCMATYGEGDPTDNAQDFYDWLQETDVDLTGVKFAVFGLGNKTYEHFNAMGKYVDQRLEQLGAQRIFELGLGDDDGNLEEDFITWREQFWPAVCEFFGVEATGEESSIRQYELVVHEDMDVAKVYTGEMGRLKSYENQKPPFDAKNPFLAAVTANRKLNQGTERHLMHLELDISDSKIRYESGDHVAVYPANDSALVNQIGEILGADLDVIMSLNNLDEESNKKHPFPCPTTYRTALTYYLDITNPPRTNVLYELAQYASEPSEQEHLHKMASSSGEGKELYLSWVVEARRHILAILQDYPSLRPPIDHLCELLPRLQARYYSIASSSKVHPNSVHICAVAVEYEAKSGRVNKGVATSWLRAKEPAGENGGRALVPMFVRKSQFRLPFKSTTPVIMVGPGTGIAPFMGFIQERAWLREQGKEVGETLLYYGCRRSDEDYLYREELARFHKDGALTQLNVAFSREQAHKVYVQHLLKRDREHLWKLIHEGGAHIYVCGDARNMAKDVQNTFYDIVAEFGPMEHTQAVDYVKKLMTKGRYSLDVWS

SEQ ID NO: 39 - protein, 214 aa, Artificial Sequence
  engineered apolipoprotein-His-TEV-MSP1T2-GT
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGT

SEQ ID NO: 40 - protein, 212 aa, Artificial Sequence
  engineered apolipoprotein-MSP1RC12′
MGHHHHHHIEGCLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 41 - protein, 212 aa, Artificial Sequence
  engineered apolipoprotein-MSP1K90C
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQCLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 42 - protein, 212 aa, Artificial Sequence
  engineered apolipoprotein-MSP1K152C
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHACATEHLSTLSEKAKPALEDLRQGLLPVLESFKVSFLSALEEYTKKLNTQ

SEQ ID NO: 43 - protein, 110 aa, Escherichia coli
MNPYIYLGGAILAEVIGTTLMKFSEGFTRLWPSVGTIICYCASFWLLAQTLAYIPTGIAYAIWSGVGIVLISLLSWGFFGQRLDLPAIIGMMLICAGVLIINLLSRSTPH

SEQ ID NO: 44 - protein, 262 aa, Halobacteriun halobium
MLELLPTAVEGVSQAQITGRPEWIWLALGTALMGLGTLYFLVKGMGVSDPDAKKFYAITTLVPAIAFTMYLSMLLGYGLTMVPFGGEQNPIYWARYADWLFTTPLLLLDLALLVDADQGTILALVGADGIMIGTGLVGALTKVYSYRFVWWAISTAAMLYILYVLFFGFTSKAESMRPEVASTFKVLRNVTVVLWSAYPVVWLIGSEGAGIVPLNIETLLFMVLDVSAKVGFGLILLRSRAIFGEAEAPEPSAGDGAAATSD

SEQ ID NO: 45 - protein, 155 aa, Homo Sapiens
MQPSPPPTELVPSERAVVLLSCALSALGSGLLVATHALWPDLRSRARRLLLFLSLADLLSAASYFYGVLQNFAGPSWDCVLQGALSTFANTSSFFWTVAIALYLYLSIVRAARGPRTDRLLWAFHVVRWVAVALLFQEPPTQADPSRSCPPRGRV

SEQ ID NO: 46 - protein, 486 aa, Homo Sapiens
MRGRGSQQQQPTRRQGQKLPSPSPAGKYESAQPGGTQPEPGLGARMAIHKALVMCLGLPLFLFPGAWAQGHVPPGCSQGLNPLYYNLCDRSGAWGIVLEAVAGAGIVTTFVLTIILVASLPFVQDTKKRSLLGTQVFFLLGTLGLFCLVFACVVKPDFSTCASRRFLFGVLFAICFSCLAAHVFALNFLARKNHGPRGWVIFTVALLLTLVEVIINTEWLIITLVRGSGEGGPQGNSSAGWAVASPCAIANMDFVMALIYVMLLLLGAFLGAWPALCGRYKRWRKHGVFVLLTTATSVAIWVVWIVMYTYGNKQHNSPTWDDPTLAIALAANAWAFVLFYVIPEVSQVTKSSPEQSYQGDMYPTRGVGYETILKEQKGQSMFVENKAFSMDEPVAAKRPVSPYSGYNGQLLTSVYQPTEMALMHKVPSEGAYDIILPRATANSQVMGSANSTLRAEDMYSAQSHQAATPPKDGKNSQVFRNPYVWD

SEQ ID NO: 47 - protein, 422 aa, Homo Sapiens
MDVLSPGQGNNTTSPPAPFETGGNTTGISDVTVSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSDPDACTISKDHGYTIYSTFGAFYIPLLLMLVLYGRIFRAARFRIRKTVKKVEKTGADTRHGASPAPQPKKSVNGESGSRNWRLGVESKAGGALCANGAVRQGDDGAALEVIEVHRVGNSKEHLPLPSEAGPTPCAPASFERKNERNAEAKRKMALARERKTVKTLGIIMGTFILCWLPFFIVALVLPFCESSCHMPTLLGAIINWLGYSNSLLNPVIYAYFNKDFQNAFKKIIKCKFCRQ

SEQ ID NO: 48 - protein, 442 aa, Homo Sapiens
MQPPPSLCGRALVALVLACGLSRIWGEERGFPPDRATPLLQTAEIMTPPTKTLWPKGSNASLARSLAPAEVPKGDRTAGSPPRTISPPPCQGPIEIKETFKYINTVVSCLVFVLGIIGNSTLLRIIYKNKCMRNGPNILIASLALGDLLHIVIDIPINVYKLLAEDWPFGAEMCKLVPFIQKASVGITVLSLCALSIDRYRAVASWSRIKGIGVPKWTAVEIVLIWVVSVVLAVPEAIGFDIITMDYKGSYLRICLLHPVQKTAFMQFYKTAKDWWLFSFYFCLPLAITAFFYTLMTCEMLRKKSGMQIALNDHLKQRREVAKTVFCLVLVFALCWLPLHLSRILKLTLYNQNDPNRCELLSFLLVLDYIGINMASLNSCINPIALYLVSKRFKNCFKSCLCCWCQSFEEKQSLEEKQSCLKFKANDHGYDNFRSSNKYSSS

SEQ ID NO: 49 - protein, 370 aa, Homo Sapiens
MEPLFPAPFWEVIYGSHLQGNLSLLSPNHSLLPPHLLLNASHGAFLPLGLKVTIVGLYLAVCVGGLLGNCLVMYVILRHTKMKTATNIYIFNLALADTLVLLTLPFQGTDILLGFWPFGNALCKTVIAIDYYNMFTSTFTLTAMSVDRYVAICHPIRALDVRTSSKAQAVNVAIWALASVVGVPVAIMGSAQVEDEEIECLVEIPTPQDYWGPVFAICIFLFSFIVPVLVISVCYSLMIRRLRGVRLLSGSREKDRNLRRITRLVLVVVAVFVGCWTPVQVFVLAQGLGVQPSSETAVAILRFCTALGYVNSCLNPILYAFLDENFKACFRKFCCASALRRDVQVSDRVRSIAKDVALACKTSETVPRPA

SEQ ID NO: 50 - protein, 466 aa, Homo Sapiens
MNNSTNSSNNSLALTSPYKTFEVVFIVLVAGSLSLVTIIGNILVMVSIKVNRHLQTVNNYFLFSLACADLIIGVFSMNLYTLYTVIGYWPLGPVVCDLWLALDYVVSNASVMNLLIISFDRYFCVTKPLTYPVKRTTKMAGMMIAAAWVLSFILWAPAILFWQFIVGVRTVEDGECYIQFFSNAAVTFGTAIAAFYLPVIIMTVLYWHISRASKSRIKKDKKEPVANQDPVSPSLVQGRIVKPNNNNMPSSDDGLEHNKIQNGKAPRDPVTENCVQGEEKESSNDSTSVSAVASNMRDDEITQDENTVSTSLGHSKDENSKQTCIRIGTKTPKSDSCTPTNTTVEVVGSSGQNGDEKQNIVARKIVKMTKQPAKKKPPPSREKKVTRTILAILLAFIITWAPYNVMVLINTFCAPCIPNTVWTIGYWLCYINSTINPACYALCNATFKKTFKHLLMCHYKNIGATR

SEQ ID NO: 51 - protein, 397 aa, Homo Sapiens
MAPNGTASSFCLDSTACKITITVVLAVLILITVAGNVVVCLAVGLNRRLRNLTNCFIVSLAITDLLLGLLVLPFSAIYQLSCKWSFGKVFCNIYTSLDVMLCTASILNLFMISLDRYCAVMDPLRYPVLVTPVRVAISLVLIWVISITLSFLSIHLGWNSRNETSKGNHTTSKCKVQVNEVYGLVDGLVTFYLPLLIMCITYYRIFKVARDQAKRINHISSWKAATIREHKATVTLAAVMGAFIICWFPYFTAFVYRGLRGDDAINEVLEAIVLWLGYANSALNPILYAALNRDFRTGYQQLFCCRLANRNSHKTSLRSNASQLSRTQSREPRQQEEKPLKLQVWSGTEVTAPQGATDRPWLCLPECWSVELTHSFIHLFIHSFANIHPIPTTCQEL

SEQ ID NO: 52 - protein, 446 aa, Homo Sapiens
MRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKQIRRIAALERAAVHAKNCQTTTGNGKPVECSQPESSFKMSFKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 53 - protein, 325 aa, Homo Sapiens
MNSSFHLHFLDLNLNATEGNLSGPNVKNKSSPCEDMGIAVEVFLTLGVISLLENILVIGAIVKNKNLHSPMYFFVCSLAVADMLVSMSSAWETITIYLLNNKHLVIADAFVRHIDNVFDSMICISVVASMCSLLAIAVDRYVTIFYALRYHHIMTARRSGAIIAGIWAFCTGCGIVFILYSESTYVILCLISMFFAMLFLLVSLYIHMFLLARTHVKRIAALPGASSARQRTSMQGAVTVTMLLGVFTVCWAPFFLHLTLMLSCPQNLYCSRFMSHFNMYLILIMCNSVMDPLIYAFRSQEMRKTFKEIICCRGFRIACSFPRRD

SEQ ID NO: 54 - protein, 415 aa, Homo Sapiens
MGGHPQLRLVKALLLLGLNPVSASLQDQHCESLSLASNISGLQCNASVDLIGTCWPRSPAGQLVVRPCPAFFYGVRYNTTNNGYRECLANGSWAARVNYSECQEILNEEKKSKVHYHVAVIINYLGHCISLVALLVAFVLFLRLRSIRCLRNIIHWNLISAFILRNATWFVVQLTMSPEVHQSNVGWCRLVTAAYNYFHVTNFFWMFGEGCYLHTAIVLTYSTDRLRKWMFICIGWGVPFPIIVAWAIGKLYYDNEKCWFGKRPGVYTDYIYQGPMILVLLINFIFLFNIVRILMTKLRASTTSETIQYRKAVKATLVLLPLLGITYMLFFVNPGEDEVSRVVFIYFNSFLESFQGFFVSVFYCFLNSEVRSAIRKRWHRWQDKHSIRARVARAMSIPTSPTRVSFHSIKQSTAV

SEQ ID NO: 55 - protein, 422 aa, Homo Sapiens; identical to SEQ ID NO: 47

SEQ ID NO: 56 - protein, 460 aa, Homo Sapiens
MNTSAPPAVSPNITVLAPGKGPWQVAFIGITTGLLSLATVTGNLLVLISFKVNTELKTVNNYFLLSLACADLIIGTFSMNLYTTYLLMGHWALGTLACDLWLALDYVASNASVMNLLLISFDRYFSVTRPLSYRAKRTPRRAALMIGLAWLVSFVLWAPAILFWQYLVGERTVLAGQCYIQFLSQPIITFGTAMAAFYLPVTVMCTLYWRIYRETENRARELAALQGSETPGKGGGSSSSSERSQPGAEGSPETPPGRCCRCCRAPRLLQAYSWKEEEEEDEGSMESLTSSEGEEPGSEVVIKMPMVDPEAQAPTKQPPRSSPNTVKRPTKKGRDRAGKGQKPRGKEQLAKRKTFSLVKEKKAARTLSAILLAFILTWTPYNIMVLVSTFCKDCVPETLWELGYWLCYVNSTINPMCYALCNKAFRDTFRLLLLCRWDKRRWRKIPKRPGSVHRTPSRQC

SEQ ID NO: 57 - protein, 80 aa, Homo Sapiens
MGRAMVARLGLGLLLLALLLPTQIYSSETTTGTSSNSSQSTSNSGLAPNPTNATTKAAGGALQSTASLFVVSLSLLHLYS

SEQ ID NO: 58 - protein, 78 aa, Homo Sapiens
MYGKIIFVLLLSGIVSISASSTTGVAMHTSTSSSVTKSYISSQTNGITLINWWAMARVIFEVMLVVVGMIILISYCIR

SEQ ID NO: 59 - protein, 91 aa, Homo Sapiens
MYGKIIFVLLLSEIVSISALSTTEVAMHTSTSSSVTKSYISSQTNGETGQLVHRFTVPAPVVIILIILCVMAGIIGTILLISYSIRRLIKA

SEQ ID NO: 60 - protein, 99 aa, Homo Sapiens
MDNVQPKIKHRPFCFSVKGHVKMLRLDIINSLVTTVFMLIVSVLALIPETTTLTVGGGVFALVTAVCCLADGALIYRKLLFNPSGPYQKKPVHEKKEVL

SEQ ID NO: 61 - protein, 150 aa, Homo Sapiens [sequence omitted]

SEQ ID NO: 62 - protein, 155 aa, Mus musculus
MADLRQLMDNEVLMAFTSYATIILTKMMFMSSATAFQRITNKVFANPEDCAGFGKVENAKKFVRTDEKVERVRRAHLNDLENIVPFLGIGLLYSLSGPDLSTALMHFRIFVGARIYHTIAYLTPLPQPNRGLAFFVGYGVTLSMAYRLLRSRLYL

SEQ ID NO: 63 - protein, 158 aa, Homo Sapiens
MVPRSTSLTLIVFLFHRLSKAPGKMVENSPSPLPERAIYGFVLFLSSQFGFILYLVWAFIPESWLNSLGLTYWPQKYWAVALPVYLLIAIVIGYVLLFGINMMSTSPLDSIHTITDNYAKNQQQKKYQEEAIPALRDISISEVNQMFFLAAKELYTKN

SEQ ID NO: 64 - protein, 162 aa, Homo Sapiens
METLPASWVLTLLCLGSHLLQAVISTTVIPSCIPGESEDNCTALVQMEDDPRVAQVQITKCSSDMDGYCLHGQCIYLVDMREKFCRCEVGYTGLRCEHFFLTVHQPLSKEYVALTVILIFLFLIITAGCIYYFCRWYKNRKSKKSREEYERVTSGDPVLPQV

SEQ ID NO: 65 - protein, 169 aa, Homo Sapiens
MTAGRRMEMLCAGRVPALLLCLGFHLLQAVLSTTVIPSCIPGESSDNCTALVQTEDNPRVAQVSITKCSSDMNGYCLHGQCIYLVDMSQNYCRCEVGYTGVRCEHFFLTVHQPLSKEYVALTVILIILFLITVVGSTYYFCRWYRNRKSKEPKKEYERVTSGDPELPQV

SEQ ID NO: 66 - protein, 185 aa, Homo Sapiens
MARGAALALLLFGLLGVLVAAPDGGFDLSDALPDNENKKPTAIPKKPSAGDDFDLGDAVVDGENDDPRPPNPPKPMPNPNPNHPSSSGSFSDADLADGVSGGEGKGGSDGGGSHRKEGEEADAPGVIPGIVGAVVVAVAGAISSFIAYQKKKLCFKENAEQGEVDMESHRNANAEPAVQRTLLEK

SEQ ID NO: 67 - protein, 176 aa, Mus musculus
MALWRAYQRALAAHPWKVQVLTAGSLMGVGDMISQQLVERRGLQQHQAGRTLTMVSLGCGFVGPVVGGWYKVLDHLIPGTTKVHALKKMLLDQGGFAPCFLGCFLPLVGILNGMSAQDNWAKLKRDYPDALITNYYLWPAVQLANFYLVPLHYRLAVVQCVAIVWNSYLSWKAHQF

SEQ ID NO: 68 - protein, 176 aa, Mus musculus
MALWRAYQRALAAHPWKVQVLTAGSLMGLGDIISQQLVERRGLQEHQRGRTLTMVSLGCGFVGPVVGGWYKVLDRFIPGTTKVDALKKMLLDQGGFAPCFLGCFLPLVGALNGLSAQDNWAKLQRDYPDALITNYYLWPAVQLANFYLVPLHYRLAVVQCVAVIWNSYLSWKAHRL

SEQ ID NO: 69 - protein, 194 aa, Homo Sapiens
MAAAAPNAGGSAPETAGSAEAPLQYSLLLQYLVGDKRQPRLLEPGSLGGIPSPAKSEEQKMIEKAMESCAFKAALACVGGFVLGGAFGVFTAGIDTNVGFDPKDPYRTPTAKEVLKDMGQRGMSYAKNFAIVGAMFSCTECLIESYRGTSDWKNSVISGCITGGAIGFRAGLKAGAIGCGGFAAFSAAIDYYLR

SEQ ID NO: 70 - protein, 188 aa, Homo Sapiens
MAGLSRQLCALSHPKKAAETQTAEPGGAHAVCSRHPVRVKGLEGSEMESARENIDLQPGSSDPRSQPINLNHYATKKSVAESMLDVALFMSNAMRLKAVLEQGPSSHYYTTLVTLISLSLLLQVVIGVLLVVIARLNLNEVEKQWRLNQLNNAATILVFFTVVINVFITAFGAHKTGFLAARASRNPL

SEQ ID NO: 71 - protein, 117 aa, Homo Sapiens [sequence omitted]

SEQ ID NO: 72 - protein, 231 aa, Homo Sapiens
MDPEHAKPESSEAPSGNLKQPETAAALASSGSVVSSVPKAQRNISAKTAPRKHPAVSIRSAQSAAAARPQGSEGTAPSRKATTRPPPKPTLPPPTPSAHTESKLLNEMAIKERVEGRAKVPYKFRDSLKRFSFSPTGMLKILRLSLILGALACFIITQANESFITITSLEICIVVFFILIYVLTLHHLLTYLHWPLLDLTNSIITAVFLSVVAILAMQEKKRRHLLYVGGR

SEQ ID NO: 73 - protein, 138 aa, Homo Sapiens
MISLTDTQKIGMGLTGFGVFFLFFGMILFFDKALLAIGNVLFVAGLAFVIGLERTFRFFFQKHKMKATGFFLGGVFVVLIGWPLIGMIFEIYGFFLLFRGFFPVVVGFIRRVPVLGSLLNLPGIRSFVDKVGESNNMV

SEQ ID NO: 74 - protein, 150 aa, Homo Sapiens
MKDEVALLAAVTLLGVLLQAYFSLQVISARRAFRVSPPLTTGPPEFERVYRAQVNCSEYFPLFLATLWVAGIFFHEGAAALCGLVYLFARLRYFQGYARSAQLRLAPLYASARALWLLVALAALGLLAHFLPAALRAALLGRLRTLLPWA

SEQ ID NO: 75 - protein, 152 aa, Homo Sapiens
MELPAVNLKVILLGHWLLTTWGCIVFSGSYAWANFTILALGVWAVAQRDSIDAISMFLGGLLATIFLDIVHISIFYPRVSLTDTGRFGVGMAILSLLLKPLSCCFVYHMYRERGGFLGSSQDRSAYQTIDSAEAPADPFAVPEGRSQDARGY

SEQ ID NO: 76 - protein, 161 aa, Homo Sapiens
MDQETVGNVVLLAIVTLISVVQNGFFAHKVEHESRTQNGRSFQRTGTLAFERVYTANQNCVDAYPTFLAVLWSAGLLCSQVPAAFAGLMYLFVRQKYFVGYLGERTQSTPGYIFGKRIILFLFLMSVAGIFNYYLIFFFGSDFENYIKTISTTISPLLLIP

SEQ ID NO: 77 - protein, 382 aa, Homo Sapiens
MAEQTYSWAYSLVDSSQVSTFLISILLIVYGSFRSLNMDFENQDKEKDSNSSSGSFNGNSTNNSIQTIDSTQALFLPIGASVSLLVMFFFFDSVQVVFTICTVLATIAFAFLLLPMCQYLTRPCSPQNKISFGCCGRFTAAELLSFSLSVMLVLIWVLTHWLLMDALAMGLCVAMIAFVRLPSLKVSCLLLSGLLIYDVFWVFFSAYIFNSNVMVKVATQPADNPLDVLSRKLHLGPNVGRDVPRLSLPGKLVFPSSTGSHFSMLGIGDIVMPGLLLCFVLRYDNYKKQASGDSCGAPGPANISGRMQKVSYFHCTLIGYFVGLLTATVASRIHRAAQPALLYLVPFTLLPLLTMAYLKGDLRRMWSEPFHSKSSSSRFLEV

SEQ ID NO: 78 - protein, 131 aa, Homo Sapiens
MAGVKALVALSFSGAIGLTFLMLGCALEDYGVYWPLFVLIFHAISPIPHFIAKRVTYDSDATSSACRELAYFFTTGIVVSAFGFPVILARVAVIKWGACGLVLAGNAVIFLTIQGFFLIFGRGDDFSWEQW

SEQ ID NO: 79 - protein, 152 aa, Homo Sapiens
MAVLSKEYGFVLLTGAASFIMVAHLAINVSKARKKYKVEYPIMYSTDPENGHIFNCIQRAHQNTLEVYPPFLFFLAVGGVYHPRIASGLGLAWIVGRVLYAYGYYTGEPSKRSRGALGSIALLGLVGTTVCSAFQHLGWVKSGLGSGPKCCH

SEQ ID NO: 80 - protein, 351 aa, Homo Sapiens
MLETLRERLLSVQQDFTSGLKTLSDKSREAKVKSKPRTVPFLPKYSAGLELLSRYEDTWAALHRRAKDCASAGELVDSEVVMLSAHWEKKKTSLVELQEQLQQLPALIADLESMTANLTHLEASFEEVENNLLHLEDLCGQCELERCKHMQSQQLENYKKNKRKELETFKAELDAEHAQKVLEMEHTQQMKLKERQKFFEEAFQQDMEQYLSTGYLQIAERREPIGSMSSMEVNVDMLEQMDLMDISDQEALDVFLNSGGEENTVLSPALGPESSTCQNEITLQVPNPSELRAKPPSSSSTCTDSATRDISEGGESPVVQSDEEEVQVDTALATSHTDREATPDGGEDSDS

SEQ ID NO: 81 - protein, 178 aa, Homo Sapiens
MSEVRLPPLRALDDFVLGSARLAAPDPCDPQRWCHRVINNLLYYQTNYLLCFGIGLALAGYVRPLHTLLSALVVAVALGVLVWAAETRAAVRRCRRSHPAACLAAVLAVGLLVLWVAGGACTFLFSIAGPVLLILVHASLRLRNLKNKIENKIESIGLKRTPMGLLLEALGQEQEAGS

SEQ ID NO: 82 - protein, 216 aa, Homo Sapiens
MGKAAAAVAFGAEVGVRLALFAAFLVTELLPPFQRLIQPEEMWLYRNPYVEAEYFPTKPMFVIAFLSPLSLIFLAKFLKKADTRDSRQACLAASLALALNGVFTNTIKLIVGRPRPDFFYRCFPDGLAHSDLMCTGDKDVVNEGRKSFPSGHSSFAFAGLAFASFYLAGKLHCFTPQGRGKSWRFCAFLSPLLFAAVIALSRTCDYKHHWQGPFKW

SEQ ID NO: 83 - protein, 185 aa, Homo Sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 84 - protein, 112 aa, Homo Sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKEGYQDYEPEA

SEQ ID NO: 85 - protein, 140 aa, Homo Sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 86 - protein, 134 aa, Homo Sapiens
MDVFMKGLSMAKEGVVAAAEKTKQGVTEAAEKTKEGVLYVGSKTREGVVQGVASVAEKTKEQASHLGGAVFSGAGNIAAATGLVKREEFPTDLKPEEVAQEAAEEPLIEPLMEPEGESYEDPPQEEYQEYEPEA

SEQ ID NO: 87 - protein, 134 aa, Homo Sapiens; identical to SEQ ID NO: 86

SEQ ID NO: 88 - protein, 127 aa, Homo Sapiens
MDVFKKGFSIAKEGVVGAVEKTKQGVTEAAEKTKEGVMYVGAKTKENVVQSVTSVAEKTKEQANAVSEAVVSSVNTVATKTVEEAENIAVTSGVVRKEDLRPSAPQQEGVASKEKEEVAEEAQSGGD

SEQ ID NO: 89 - protein, 262 aa, Danio rerio
MKFVALALTLLLALGSQANLFQADAPTQLEHYKAAALVYLNQVKDQAEKALDNLDGTDYEQYKLQLSESLTKLQEYAQTTSQALTPYAETISTQLMENTKQLRERVMTDVEDLRSKLEPHRAELYTALQKHIDEYREKLEPVFQEYSALNRQNAEQLRAKLEPLMDDIRKAFESNIEETKSKVVPMVEAVRTKLTERLEDLRTMAAPYAEEYKEQLVKAVEEAREKIAPHTQDLQTRMEPYMENVRTTFAQMYETIAKAIQA